(19) United States
(12) Patent Application Publication
(10) Pub. No.: US 2020/0121745 A1
(43) Pub. Date: Apr. 23, 2020

(54) COMPOSITIONS AND METHODS FOR CANCER THERAPY WITH DENGUE VIRUS AND DENDRITIC CELLS

(71) Applicant: PrimeVax Immuno-Oncology, Inc., Orange, CA (US)

(72) Inventors: Bruce W. LYDAY, Orange, CA (US); Tony CHEN, Orange, CA (US)

(21) Appl. No.: 16/714,370

(22) Filed: Dec. 13, 2019

Related U.S. Application Data
(63) Continuation of application No. PCT/US18/37616, filed on Jun. 14, 2018.
(60) Provisional application No. 62/520,345, filed on Jun. 15, 2017.

(51) Int. Cl. A61K 35/76; C12N 7/00; A61K 35/15; C12N 5/0784; A61P 35/00

(52) U.S. Cl. CPC A61K 35/76; C12N 7/00; A61K 35/15; C12N 2770/24121; A61P 35/00; C12N 2770/24133; C12N 5/0639

(57) ABSTRACT

Described herein are compositions and methods for treating a disease, particularly a melanoma, with a Dengue Virus and, optionally, primed dendritic cells recognizing a tumor antigen. Lysis protocols are described where the lysis does not result in complete or less than complete lysis of cells in order to provide cell surface molecules maintained in a cell surface-embedded state. Non-lethal Dengue virus strains are also provided for therapeutic purposes.

### CROSS-REFERENCE

This application is a continuation of PCT/US2018/037616 filed Jun. 14, 2018, which claims the benefit of U.S. Provisional Patent Application No. 62/520,345 filed Jun. 15, 2017, which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jun. 14, 2018, is named 48253-707_301_SL.txt and is 45,748 bytes in size.

### BACKGROUND

Immunotherapy, unlike cytotoxic drugs, radiation, and surgery, stimulates the immune system to recognize and kill tumor cells. Numerous attempts have been made in stimulating the immune system to recognize and destroy tumor cells. These have been met with limited success due to the self-identity of peptides selected as target for immunotherapy, lack of immune activation, adverse events, and/or tumor immune evasion mechanisms.

The ability of current cellular therapies, e.g., dendritic cell therapies, to induce durable, complete responses in advanced cancer patients is low (5-10% in the most immunogenic cancer types, lower in others). Often, dendritic cell therapies produce less than desirable results because of low activation (e.g., not enough immune cells to adequately kill all cancer cells), low targeting (e.g., healthy cells are killed and/or tumor cells are not killed), or an immunosuppressed tumor microenvironment, limiting drug efficacy. Thus there is a need for improved immunotherapies to treat cancer.

Tumors, by virtue of their high mitotic and cellular metabolic rates, are often oxygen deficient. This oxygen deficiency leads to higher utilization of anaerobic pathways to generate adenosine triphosphate (ATP), with the result of higher levels of lactate, and lower pH within the cytoplasm and nucleus. Thus there is a need for targeting and eradicating these low-perfusion tumor sites with high genetic plasticity.

### BRIEF SUMMARY

Provide wherein are methods for treatment or reduction of a melanoma, comprising: administering Dengue virus to a subject in need thereof, wherein the subject has melanoma; and administering primed dendritic cells to the subject, wherein the primed dendritic cells are produced by contacting dendritic cells with a tumor antigen. Further provided herein are methods wherein the melanoma is advanced melanoma. Further provided herein are methods wherein the melanoma is advanced and is Stage III or Stage IV melanoma. Further provided herein are methods comprising obtaining the dendritic cells from the subject at least a week prior to administering the dose of Dengue virus. Further provided herein are methods wherein the Dengue virus is administered in an amount between 10⁴ pfu and 10⁸ pfu. Further provided herein are methods wherein the Dengue virus is administered in an amount between 10⁵ pfu and 10⁷ pfu. Further provided herein are methods wherein the Dengue virus is administered in a concentration of 10,000 PFU/mL to 90,000 PFU/mL. Further provided herein are methods wherein the Dengue virus is administered in a concentration of about 30,000 PFU/mL. Further provided herein are methods comprising administering primed dendritic cells 4 days to 10 days after administering the dose of Dengue virus. Further provided herein are methods wherein the Dengue virus is administered subcutaneously. Further provided herein are methods wherein the Dengue virus is administered via intratumoral injection. Further provided herein are methods comprising administering primed dendritic cells when the subject presents a febrile symptom. Further provided herein are methods comprising administering primed dendritic cells when the subject has reached a temperature of 101° F. Further provided herein are methods comprising administering a first aliquot of primed dendritic cells to the subject at a first time and a second aliquot of primed dendritic cells at a second time. Further provided herein are methods wherein the first time and the second time are separated by up to 30 days. Further provided herein are methods wherein the first time and the second time are separated by about 3 days. Further provided wherein are methods wherein the number of primed dendritic cells in the first aliquot of primed dendritic cells is 10⁴ cells to 10⁸ cells. Further provided herein are methods wherein the total number of primed dendritic cells in each of the first aliquot of primed dendritic cells and second aliquot of primed dendritic cells is 10⁶ cells to 10⁹ cells. Further provided herein are methods wherein the dendritic cells are allogeneic to the subject. Further provided herein are methods wherein the dendritic cells are autologous to the subject. Further provided herein are methods comprising obtaining the dendritic cells from the subject. Further provided herein are methods comprising contacting the dendritic cells with tumor lysate from the subject. Further provided herein are methods wherein the primed dendritic cells produce at least about 16 ng/mL IL-12p70. Further provided herein are methods wherein the primed dendritic cells produce at least about 29 ng/mL IL-12p70. Further provided herein are methods wherein the Dengue virus is a serotype 1, 2, 3, 4 or 5. Further provided herein are methods wherein the Dengue virus a DENV2 #1710. Further provided herein are methods wherein the Dengue virus a DENV1 #45AZ5. Further provided herein are methods wherein the Dengue virus is S16803, HON 1991 C, HON 1991 D, HON 1991 B, HON 1991 A, SAL 1987, TRI 1981, PR 1969, IND 1957, TRI 1953, TSVO1, DS09-280106, DS31-291005, 1349, GD01/03, 44, 43, China 04, FJ11/99, FJ-10, QHD13CAIQ, CO/BID-V3358, FJ/UH21/1971, GU/BID-V2950, American Asian, GWL18, IN/BID-V2961, Od2112, RR44, 1392, 1016DN, 1017DN, 1070DN, 98900663DHF, BA05i, 1022DN, NGC, Pak-L-2011, Pak-K-2009, Pak-M-2011, PakL-2013, Pak-L-2011, Pak-L-2010, Pak-L-2008, PE/NFI1159, PE/IQA 2080, SG/D2Y98P-PP1, SG/05K3295DK1, LK/BID/V2421, LK/BID-V2422, LK/BID-V2416, 1222-DF-06, TW/BID-V5056, TH/BID-V3357, US/BID-V5412, US/BID-V5055, IQT1797, VN/BID-V735, US/Hawaii/1944, CH53489, or 341750.

Provided herein are methods for treatment or reduction of a melanoma, comprising: administering DENV1 #45AZ5 to a subject in need thereof, wherein the subject has melanoma; obtaining dendritic cells from the subject; contacting the dendritic cells with a tumor antigen from the subject to generate primed dendritic cells; and administering the primed dendritic cells to the subject. Further provided herein are methods wherein the melanoma is advanced melanoma. Further provided herein are methods wherein the melanoma is advanced and is Stage III or Stage IV melanoma. Further provided herein are methods wherein the DENV1 #45AZ5 is administered in an amount between 10⁴ pfu and 10⁸ pfu. Further provided herein are methods wherein the DENV1 #45AZ5 is administered in an amount between 10⁵ pfu and 10⁷ pfu. Further provided herein are methods wherein the DENV1 #45AZ5 is administered in a concentration of 10,000 PFU/mL to 90,000 PFU/mL. Further provided herein are methods wherein the DENV1 #45AZ5 is administered in a concentration of about 30,000 PFU/mL.

Provided herein are methods for treatment or reduction of a melanoma, comprising: administering DENV2 #1710 to a subject in need thereof, wherein the subject has melanoma; obtaining dendritic cells from the subject; contacting the dendritic cells with a tumor antigen from the subject to generate primed dendritic cells; and administering the primed dendritic cells to the subject. Further provided herein are methods wherein the melanoma is advanced melanoma. Further provided herein are methods wherein the melanoma is advanced and is Stage III or Stage IV melanoma. Further provided herein are methods wherein the DENV2 #1710 is administered in an amount between 10⁴ pfu and 10⁸ pfu. Further provided herein are methods wherein the DENV2 #1710 is administered in an amount between 10⁵ pfu and 10⁷ pfu. Further provided herein are methods wherein the DENV2 #1710 is administered in a concentration of 10,000 PFU/mL to 90,000 PFU/mL. Further provided herein are methods wherein the DENV2 #1710 is administered in a concentration of about 30,000 PFU/mL.

### BRIEF DESCRIPTION OF THE FIGURES

FIG. 1 depicts an exemplary method of treatment with Dengue virus and dendritic cells.

FIG. 2 is a plot of corresponding to the number of lung metastases from melanoma cells in mice under various treatment conditions. The patterned bars depict the mean number of lung metastases for each condition.

FIG. 3 is a plot of corresponding to the number of lung metastases from melanoma cells in mice under various treatment conditions. The patterned bars depict the mean number of lung metastases for each condition.

FIG. 4 is a plot of flow cytometry data confirming isolation of CD14+ monocytes.

FIG. 5 is a plot of protein expression data for IL-12p70 expressed by DCs produced by methods disclosed herein relative to that of DCs produced by comparator methods.

FIG. 6 is a plot of cytotoxicity of Dengue Virus induced supernatant on a melanoma cell line (FEMX cells) in the presence of cytotoxic T lymphocytes. The Y axis is a percentage of cells death relative to total cells.

FIG. 7 is a plot of cytotoxicity of Dengue Virus induced supernatant on a melanoma cell line (624.28 cells) in the presence of cytotoxic T lymphocytes. The Y axis is a percentage of cells death relative to total cells.

FIG. 8 is a plot of cytotoxicity of Dengue Virus induced supernatant and natural killer cells on a melanoma cell line (FEMX cells). The Y axis is a percentage of cells death relative to total cells.

FIG. 9 is a plot of cytotoxicity of Dengue Virus induced supernatant and natural killer cells on a melanoma cell line (FEMX cells). The Y axis is a percentage of cells death relative to total cells.

FIG. 10 is a plot of DV induced supernatants are cytotoxic to melanoma cell line 624.28 cells in the absence of cytotoxic T lymphocytes (CTL) or natural killer (NK) cells. The Y axis is a percentage of cells death relative to total cells.

### DETAILED DESCRIPTION

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

The term “subject” as used herein includes to mammals. Mammals include rats, mice, non-human primates, and primates, including humans.

### Cancer Therapy

Provided herein are compositions and uses thereof where the compositions have Dengue virus present in an effective amount for the treatment or reduction of a cancer in a subject in need thereof. Use of Dengue virus as described herein includes the therapeutic administration of Dengue virus to treat various conditions, such as cancer, in a subject. Further provided herein are methods of treating cancer by administering to a subject an effective amount of Dengue virus wherein the Dengue virus is able to treat, stabilize, or reduce a cancer in the treated subject as compared to an untreated subject. Further provided is a composition comprising a Dengue virus that can also be used as an adjuvant for cancer therapy. In some instance, the Dengue virus is part of a combination therapy for treatment of cancer. The Dengue virus therapy is administered in conjunction with various anti-cancer therapies such as those combining physiological (hyperthermic reduction of tumor perfusion), immunological (activation of effector cells of the adaptive and innate immune system), and apoptosis-inducing pathways (sTRAIL) to destroy or stabilize the growth of tumor cells.

### Dengue Viruses

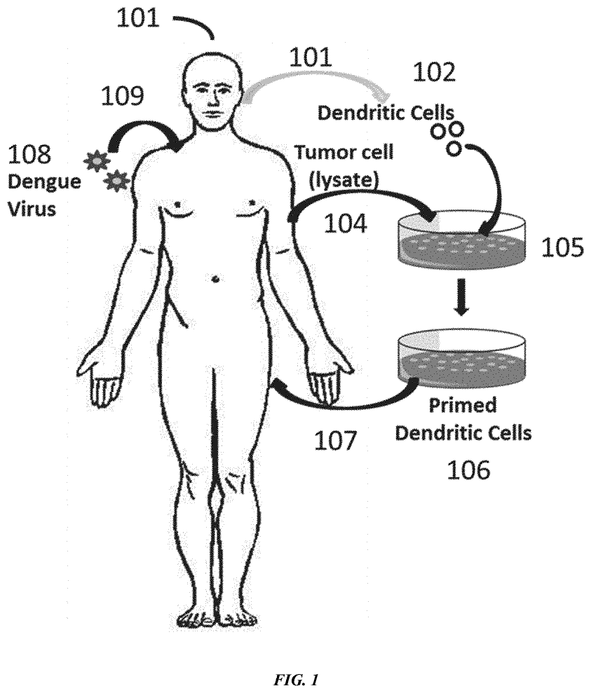

Dengue virus is useful for compositions and methods described herein as primary infections carry lower mortality than the common cold while also allowing for increased capillary permeability, and cytokine production, among other features. Provided herein are compositions for the treatment of cancer, wherein the composition comprises a Dengue virus in an effective amount for depletion or reduction of cancer in a subject in need thereof. (FIGURE. 1) Also provided herein are methods for treatment of cancer, comprising administering to a subject in need thereof, an effective amount of a Dengue virus for depletion or reduction of a cancer. Also provided herein are methods for the stabilization of cancer, comprising administering to a subject in need thereof, an effective amount of a Dengue virus for stabilizing or controlling growth of a cancer. Dengue viruses are Arboviruses, and are transmitted exclusively by mosquitoes of the Aedes aegypti and albopictus species. The virus has a complex life cycle involving an unidentified forest-dwelling mammalian reservoir (possibly primates), and human hosts. The female mosquito takes a blood meal from an infected person, the virus replicates to a high infectious titer (10⁵/ml) in gut epithelial cells, then is transmitted to another person when the mosquito withdraws its stylet using back pressure after another blood meal. Dengue epidemics infect 50 million persons annually, with several thousand deaths, usually children with inadequate treatment of secondary infection-related shock.

The Dengue virus genome encodes structural proteins, capsid protein C, membrane protein M, envelope protein E, and nonstructural proteins, NS1, NS2a, NS2b, NS3, NS4a, NS4b and NS5. In some instances, the Dengue virus is a live strain of the Dengue virus. In some instances, the Dengue virus is an attenuated strain of the Dengue virus. In some instances, the Dengue virus is a weakened strain of the Dengue virus. In some instances, the Dengue virus is selected from the following serotypes of dengue virus: DENV-1, DENV-2, DENV-3, DENV-4, and DENV-5, and combinations thereof. Provided herein are methods and composition for combination therapy, comprising administering to a subject in need thereof: a Dengue virus (DV) and Dendritic Cells (DCs) primed to target tumor cells.

Dengue Viruses are positive-strand RNA viruses of the Togavirus Family, sub-family Flaviviridae, (Group B). The virus has an icosahedral geometry and is approximately 40-45 nanometers in diameter. The 11,000 base genome codes for a nucleocapsid (NC) protein, a prM membrane fusion protein, an envelope glycoprotein (E), and 5 non-structural proteins NS1-NS5. The NC protein forms the viral core, with the envelope spikes attached via the prM complex. The E glycoprotein is notable target of neutralizing antibodies, and the NS-3 and NS-4 proteins are notable targets for CD4+ and CD8+ CTLs.

The Dengue viruses make up five distinct serotypes, DENV-1 through DENV-5. The serotypes 2 and 4 are cross-neutralizing for IgG, and types 1 and 3 are also cross-neutralizing. Immunity is not complete, however, and Dengue is unique among viral infections in that a subsequent infection by a non-cross-neutralizing serotype carries an increased risk of mortality due to shock syndrome from immune hyper-activation. In some cases, a non-lethal form of a Dengue virus can be utilized. Exemplary non-lethal Dengue viruses can be of serotype 1, 2, 3, 4, or 5. For example, a non-lethal Dengue virus can be selected from Table 1. For example a Dengue Virus can be from about 50%, 60%, 70%, 80%, 90%, 95%, 96%, 97%, 98%, 99%, or up to about 100% identical in sequence homology or structural homology to any strain of Table 1.

TABLE 1
Non-lethal Dengue Virus Strains

| Serotype | Strain |
|---|---|
| I | 45AZ5 |
| II | 1710 |
| II | S16803 |
| II | HON 1991 C |
| II | HON 1991 D |
| II | HON 1991 B |
| II | HON 1991 A |
| II | SAL 1987 |
| II | TRI 1981 |
| II | PR 1969 |
| II | IND 1957 |
| II | TRI 1953 |
| II | TSV01 |
| II | DS09-280106 |
| II | DS31-291005 |
| II | 1349 |
| II | GD01/03 |
| II | 44 |
| II | 43 |
| II | China 04 |
| II | FJ11/99 |
| II | FJ-10 |
| II | QHD13CAIQ |
| II | CO/BID-V3358 |
| II | FJ/UH21/1971 |
| II | GU/BID-V2950 |
| II | American Asian |
| II | GWL18 |
| II | IN/BID-V2961 |
| II | Od2112 |
| II | RR44 |
| II | 1392 |
| II | 1016DN |
| II | 1017DN |
| II | 1070DN |
| II | 98900663DHF |
| II | BA05i |
| II | 1022DN |
| II | NGC |
| II | Pak-L-2011 |
| II | Pak-K-2009 |
| II | Pak-M-2011 |
| II | PakL-2013 |
| II | Pak-L-2011 |
| II | Pak-L-2010 |
| II | Pak-L-2008 |
| II | PE/NFI1159 |
| II | PE/IQA 2080 |
| II | SG/D2Y98P-PP1 |
| II | SG/05K3295DK1 |
| II | LK/BID/V2421 |
| II | LK/BID-V2422 |
| II | LK/BID-V2416 |
| II | 1222-DF-06 |
| II | TW/BID-V5056 |
| II | TH/BID-V3357 |
| II | US/BID-V5412 |
| II | US/BID-V5055 |
| II | IQT1797 |
| II | VN/BID-V735 |
| II | US/Hawaii/1944 |
| III | CH53489 |
| IV | 341750 |

Provided herein are compositions and methods using one more Dengue virus strains, wherein the composition comprises a Dengue virus strain of serotype 1, 2, 3, 4, or 5. In some instances, the Dengue virus is of serotype 1. In some cases, the DV is strain 45AZ5. DNA corresponding to the 45AZ5 genome, and the protein sequence are provided in Table 2.

TABLE 2
DNA and amino acid sequence of DV strain 45AZ5

| SEQ ID | |
|---|---|
| NO: | Sequence |
| 8 | AGTTGTTAGTCTACGTGGACCGACAAGAACAGTTTCGAATCGGAAGCTTGCTTAAC |
| | GTAGTTCTAACAGTTTTTTATTAGAGAGCAGATCTCTGATGAACAACCAACGGAAA |
| | AAGACGGGTCGACCGTCTTTCAATATGCTGAAACGCGCGAGAAACCGCGTGTCAAC |
| | TGTTTCACAGTTGGCGAAGAGATTCTCAAAAGGATTGCTTTCAGGCCAAGGACCCA |
| | TGAAATTGGTGATGGCTTTTATAGCATTCCTAAGATTTCTAGCCATACCTCCAACAG |
| | CAGGAATTTTGGCTAGATGGGGCTCATTCAAGAAGAATGGAGCGATCAAAGTGTTA |
| | CGGGGTTTCAAGAAAGAAATCTCAAACATGTTGAACATAATGAACAGGAGGAAAA |
| | GATCTGTGACCATGCTCCTCATGCTGCTGCCCACAGCCCTGGCGTTCCATCTGACCA |
| | CCCGAGGGGGAGAGCCGCACATGATAGTTAGCAAGCAGGAAAGAGGAAAATCACT |
| | TTTGTTTAAGACCTCTGCAGGTGTCAACATGTGCACCCTTATTGCAATGGATTTGGG |
| | AGAGTTATGTGAGGACACAATGACCTACAAATGCCCCCGGATCACTGAGACGGAA |
| | CCAGATGACGTTGACTGTTGGTGCAATGCCACGGAGACATGGGTGACCTATGGAAC |
| | ATGTTCTCAAACTGGTGAACACCGACGAGACAAACGTTCCGTCGCACTGGCACCAC |
| | ACGTAGGGCTTGGTCTAGAAACAAGAACCGAAACGTGGATGTCCTCTGAAGGCGC |
| | TTGGAAACAAATACAAAAAGTGGAGACCTGGGCTCTGAGACACCCAGGATTCACG |
| | GTGATAGCCCTTTTTCTAGCACATGCCATAGGAACATCCATCACCCAGAAAGGGAT |
| | CATTTTTATTTTGCTGATGCTGGTAACTCCATCCATGGCCATGCGGTGCGTGGGAAT |
| | AGGCAACAGAGACTTCGTGGAAGGACTGTCAGGAGCTACGTGGGTGGATGTGGTA |
| | CTGGAGCATGGAAGTTGCGTCACTACCATGGCAAAAGACAAACCAACACTGGACA |
| | TTGAACTCTTGAAGACGGAGGTCACAAACCCTGCCGTCCTGCGCAAACTGTGCATT |
| | GAAGCTAAAATATCAAACACCACCACCGATTCGAGATGTCCAACACAAGGAGAAG |
| | CCACGCTGGTGGAAGAACAGGACACGAACTTTGTGTGTCGACGAACGTTCGTGGAC |
| | AGAGGCTGGGGCAATGGTTGTGGGCTATTCGGAAAAGGTAGCTTAATAACGTGTGC |
| | TAAGTTTAAGTGTGTGACAAAACTGGAAGGAAAGATAGTCCAATATGAAAACTTA |
| | AAATATTCAGTGATAGTCACCGTACACACTGGAGACCAGCACCAAGTTGGAAATG |
| | AGACCACAGAACATGGAACAACTGCAACCATAACACCTCAAGCTCCCACGTCGGA |
| | AATACAGCTGACAGACTACGGAGCTCTAACATTGGATTGTTCACCTAGAACAGGGC |
| | TAGACTTTAATGAGATGGTGTTGTTGACAATGAAAAAAAAATCATGGCTCGTCCAC |
| | AAACAATGGTTTCTAGACTTACCACTGCCTTGGACCTCGGGGGCTTCAACATCCCA |
| | AGAGACTTGGAATAGACAAGACTTGCTGGTCACATTTAAGACAGCTCATGCAAAA |
| | AAGCAGGAAGTAGTCGTACTAGGATCACAAGAAGGAGCAATGCACACTGCGTTGA |
| | CTGGAGCGACAGAAATCCAAACGTCTGGAACGACAACAATTTTTGCAGGACACCT |
| | GAAATGCAGATTAAAAATGGATAAACTGATTTTAAAAGGGATGTCATATGTAATGT |
| | GCACAGGGTCATTCAAGTTAGAGAAGGAAGTGGCTGAGACCCAGCATGGAACTGT |
| | TCTAGTGCAGGTTAAATACGAAGGAACAGATGCACCATGCAAGATCCCCTTCTCGT |
| | CCCAAGATGAGAAGGGAGTAACCCAGAATGGGAGATTGATAACAGCCAACCCCAT |
| | AGTCACTGACAAAGAAAAACCAGTCAACATTGAAGCGGAGCCACCTTTTGGTGAG |
| | AGCTACATTGTGGTAGGAGCAGGTGAAAAAGCTTTGAAACTAAGCTGGTTCAAGA |
| | AGGGAAGCAGTATAGGGAAAATGTTTGAAGCAACTGCCCGTGGAGCACGAAGGAT |
| | GGCCATCCTGGGAGACACTGCATGGGACTTCGGTTCTATAGGAGGGGTGTTCACGT |
| | CTGTGGGAAAACTGATACACCAGATTTTTGGGACTGCGTATGGAGTTTTGTTCAGC |
| | GGTGTTTCTTGGACCATGAAGATAGGAATAGGGATTCTGCTGACATGGCTAGGATT |
| | AAACTCAAGGAGCACGTCCCTTTCAATGACGTGTATCGCAGTTGGCATGGTCACAC |
| | TGTACCTAGGAGTCATGGTTCAGGCGGACTCGGGATGTGTAATCAACTGGAAAGGC |
| | AGAGAACTCAAATGTGGAAGCGGCATTTTTGTCACCAATGAAGTCCACACCTGGAC |
| | AGAGCAATATAAATTCCAGGCCGACTCCCCTAAGAGACTATCAGCGGCCATTGGGA |
| | AGGCATGGGAGGAGGGTGTGTGTGGAATTCGATCAGCCACTCGTCTCGAGAACATC |
| | ATGTGGAAGCAAATATCAAATGAATTAAACCACATCTTACTTGAAAATGACATGAA |
| | ATTTACAGTGGTCGTAGGAGACGTTAGTGGAATCTTGGCCCAAGGAAAGAAAATG |
| | ATTAGGCCACAACCCATGGAACACAAATACTCGTGGAAAAGCTGGGGAAAAGCCA |
| | AAATCATAGGAGCAGATGTACAGAATACCACCTTCATCATCGACGGCCCAAACACC |
| | CCAGAATGCCCTGATAACCAAAGAGCATGGAACATTTGGGAAGTTGAAGACTATG |
| | GATTTGGAATTTTCACGACAAACATATGGTTGAAATTGCGTGACTCCTACACTCAA |
| | GTGTGTGACCACCGGCTAATGTCAGCTGCCATCAAGGATAGCAAAGCAGTCCATGC |
| | TGACATGGGGTACTGGATAGAAAGTGAAAAGAACGAGACTTGGAAGTTGGCAAGA |
| | GCCTCCTTCATAGAAGTTAAGACATGCATCTGGCCAAAATCCCACACTCTATGGAG |
| | CAATGGAGTCCTGGAAAGTGAGATGATAATCCCAAAGATATATGGAGGACCAATA |
| | TCTCAGCACAACTACAGACCAGGATATTTCACACAAACAGCAGGGCCGTGGCACTT |
| | GGGCAAGTTAGAACTAGATTTTGATTTATGTGAAGGTACCACTGTTGTTGTGGATG |
| | AACATTGTGGAAATCGAGGACCATCTCTTAGAACCACAACAGTCACAGGAAAGAC |
| | AATCCATGAATGGTGCTGTAGATCTTGCACGTTACCCCCCCTACGTTTCAAAGGAG |
| | AAGACGGGTGCTGGTACGGCATGGAAATCAGACCAGTCAAGGAGAAGGAAGAGA |
| | ACCTAGTTAAGTCAATGGTCTCTGCAGGGTCAGGAGAAGTGGACAGTTTTTCACTA |
| | GGACTGCTATGCATATCAATAATGATCGAAGAGGTAATGAGATCCAGATGGAGCA |
| | GAAAAATGCTGATGACTGGAACATTGGCTGTGTTCCTCCTTCTCACAATGGGACAA |
| | TTGACATGGAATGATCTGATCAGGCTATGTATCATGGTTGGAGCCAACGCTTCAGA |
| | CAAGATGGGGATGGGAACAACGTACCTAGCTTTGATGGCCACTTTCAGAATGAGAC |
| | CAATGTTCGCAGTCGGGCTACTGTTTCGCAGATTAACATCTAGAGAAGTTCTTCTTC |
| | TTACAGTTGGATTGAGTCTGGTGGCATCTGTAGAACTACCAAATTCCTTAGAGGAG |
| | CTAGGGGATGGACTTGCAATGGGCATCATGATGTTGAAATTACTGACTGATTTTCA |
| | GTCACATCAGCTATGGGCTACCTTGCTGTCTTTAACATTTGTCAAAACAACTTTTTC |
| | ATTGCACTATGCATGGAAGACAATGGCTATGATACTGTCAATTGTATCTCTCTTCCC |
| | TTTATGCCTGTCCACGACTTCTCAAAAAACAACATGGCTTCCGGTGTTGCTGGGATC |
| | TCTTGGATGCAAACCACTAACCATGTTTCTTATAACAGAAAACAAAATCTGGGGAA |
| | GGAAAAGCTGGCCTCTCAATGAAGGAATTATGGCTGTTGGAATAGTTAGCATTCTT |
| | CTAAGTTCACTTCTCAAGAATGATGTGCCACTAGCTGGCCCACTAATAGCTGGAGG |
| | CATGCTAATAGCATGTTATGTCATATCTGGAAGCTCGGCCGATTTATCACTGGAGA |
| | AAGCGGCTGAGGTCTCCTGGGAAGAAGAAGCAGAACACTCTGGTGCCTCACACAA |
| | CATACTAGTGGAGGTCCAAGATGATGGAACCATGAAGATAAAGGATGAAGAGAGA |
| | GATGACACACTCACCATTCTCCTCAAAGCAACTCTGCTAGCAATCTCAGGGGTATA |
| | CCCAATGTCAATACCGGCGACCCTCTTTGTGTGGTATTTTTGGCAGAAAAAGAAAC |
| | AGAGATCAGGAGTGCTATGGGACACACCCAGCCCTCCAGAAGTGGAAAGAGCAGT |
| | CCTTGATGATGGCATTTATAGAATTCTCCAAAGAGGATTGTTGGGCAGGTCTCAAG |
| | TAGGAGTAGGAGTTTTTCAAGAAGGCGTGTTCCACACAATGTGGCACGTCACCAGG |
| | GGAGCTGTCCTCATGTACCAAGGGAAGAGACTGGAACCAAGTTGGGCCAGTGTCA |
| | AAAAAGACTTGATCTCATATGGAGGAGGTTGGAGGTTTCAAGGATCCTGGAACGC |
| | GGGAGAAGAAGTGCAGGTGATTGCTGTTGAACCGGGGAAGAACCCCAAAAATGTA |
| | CAGACAGCGCCGGGTACCTTCAAGACCCCTGAAGGCGAAGTTGGAGCCATAGCTCT |
| | AGACTTTAAACCCGGCACATCTGGATCTCCTATCGTGAACAGAGAGGGAAAAATA |
| | GTAGGTCTTTATGGAAATGGAGTGGTGACAACAAGTGGTACCTACGTCAGTGCCAT |
| | AGCTCAAGCTAAAGCATCACAAGAAGGGCCTCTACCAGAGATTGAGGACGAGGTG |
| | TTTAGGAAAAGAAACTTAACAATAATGGACCTACATCCAGGATCGGGAAAAACAA |
| | GAAGATACCTTCCAGCCATAGTCCGTGAGGCCATAAAAAGAAAGCTGCGCACGCT |
| | AGTCTTAGCTCCCACAAGAGTTGTCGCTTCTGAAATGGCAGAGGCGCTCAAGGGAA |
| | TGCCAATAAGGTATCAGACAACAGCAGTGAAGAGTGAACACACGGGAAAGGAGAT |
| | AGTTGACCTTATGTGTCACGCCACTTTCACTATGCGTCTCCTGTCTCCTGTGAGAGT |
| | TCCCAATTATAATATGATTATCATGGATGAAGCACATTTTACCGATCCAGCCAGCA |
| | TAGCAGCCAGAGGGTATATCTCAACCCGAGTGGGTATGGGTGAAGCAGCTGCGATT |
| | TTCATGACAGCCACTCCCCCCGGATCGGTGGAGGCCTTTCCACAGAGCAATGCAGT |
| | TATCCAAGATGAGGAAAGAGACATTCCTGAAAGATCATGGAACTCAGGCTATGAC |
| | TGGATCACTGATTTCCCAGGTAAAACAGTCTGGTTTGTTCCAAGCATCAAATCAGG |
| | AAATGACATTGCCAACTGTTTAAGAAAGAATGGGAAACGGGTGGTCCAATTGAGC |
| | AGAAAAACTTTTGACACTGAGTACCAGAAAACAAAAAATAACGACTGGGACTATG |
| | TTGTCACAACAGACATATCCGAAATGGGAGCAAACTTCCGAGCCGACAGGGTAAT |
| | AGACCCGAGGCGGTGCCTGAAACCGGTAATACTAAAAGATGGCCCAGAGCGTGTC |
| | ATTCTAGCCGGACCGATGCCAGTGACTGTGGCTAGCGCCGCCCAGAGGAGAGGAA |
| | GAATTGGAAGGAACCAAAATAAGGAAGGCGATCAGTATATTTACATGGGACAGCC |
| | TCTAAACAATGATGAGGACCACGCCCATTGGACAGAAGCAAAAATGCTCCTTGAC |
| | AACATAAACACACCAGAAGGGATTATCCCAGCCCTCTTTGAGCCGGAGAGAGAAA |
| | AGAGTGCAGCAATAGACGGGGAATACAGACTACGGGGTGAAGCGAGGAAAACGTT |
| | CGTGGAGCTCATGAGAAGAGGAGATCTACCTGTCTGGCTATCCTACAAAGTTGCCT |
| | CAGAAGGCTTCCAGTACTCCGACAGAAGGTGGTGCTTTGATGGGGAAAGGAACAA |
| | CCAGGTGTTGGAGGAGAACATGGACGTGGAGATCTGGACAAAAGAAGGAGAAAG |
| | AAAGAAACTACGACCCCGCTGGCTGGATGCCAGAACATACTCTGACCCACTGGCTC |
| | TGCGCGAATTCAAAGAGTTCGCAGCAGGAAGAAGAAGCGTCTCAGGTGACCTAAT |
| | ATTAGAAATAGGGAAACTTCCACAACATTTAACGCAAAGGGCCCAGAACGCCTTG |
| | GACAATCTGGTTATGTTGCACAACTCTGAACAAGGAGGAAAAGCCTATAGACACG |
| | CCATGGAAGAACTACCAGACACCATAGAAACGTTAATGCTCCTAGCTTTGATAGCT |
| | GTGCTGACTGGTGGAGTGACGTTGTTCTTCCTATCAGGAAGGGGTCTAGGAAAAAC |
| | ATCCATTGGCCTACTCTGCGTGATTGCCTCAAGTGCACTGTTATGGATGGCCAGTGT |
| | GGAACCCCATTGGATAGCGGCCTCTATCATACTGGAGTTCTTTCTGATGGTGTTGCT |
| | TATTCCAGAGCCGGACAGACAGCGCACTCCACAAGACAACCAGCTAGCATACGTG |
| | GTGATAGGTCTGTTATTCATGATATTGACAGTGGCAGCCAATGAGATGGGATTACT |
| | GGAAACCACAAAGAAGGACCTGGGGATTGGTCATGCAGCTGCTGAAAACCACCAT |
| | CATGCTGCAATGCTGGACGTAGACCTACATCCAGCTTCAGCCTGGACTCTCTATGC |
| | AGTGGCCACAACAATTATCACTCCCATGATGAGACACACAATTGAAAACACAACG |
| | GCAAATATTTCCCTGACAGCTATTGCAAACCAGGCAGCTATATTGATGGGACTTGA |
| | CAAGGGATGGCCAATATCAAAGATGGACATAGGAGTTCCACTTCTCGCCTTGGGGT |
| | GCTATTCTCAGGTGAACCCGCTGACGCTGACAGCGGCGGTATTGATGCTAGTGGCT |
| | CATTATGCCATAATTGGACCCGGACTGCAAGCAAAAGCTACTAGAGAAGCTCAAA |
| | AAAGGACAGCAGCCGGAATAATGAAAAACCCAACTGTCGACGGGATCGTTGCAAT |
| | AGATTTGGACCCTGTGGTTTACGATGCAAAATTTGAAAAACAGCTAGGCCAAATAA |
| | TGTTGTTGATACTTTGCACATCACAGATCCTCCTGATGCGGACCACATGGGCCTTGT |
| | GTGAATCCATCACACTAGCCACTGGACCTCTGACTACGCTTTGGGAGGGATCTCCA |
| | GGAAAATTCTGGAACACCACGATAGCGGTGTCCATGGCAAACATTTTTAGGGGAA |
| | GTTATCTAGCAGGAGCAGGTCTGGCCTTTTCATTAATGAAATCTCTAGGAGGAGGT |
| | AGGAGAGGCACGGGAGCCCAAGGGGAAACACTGGGAGAAAAATGGAAAAGACAG |
| | CTAAACCAATTGAGCAAGTCAGAATTCAACACTTACAAAAGGAGTGGGATTATAG |
| | AGGTGGATAGATCTGAAGCCAAAGAGGGGTTAAAAAGAGGAGAAACGACTAAAC |
| | ACGCAGTGTCGAGAGGAACGGCCAAACTGAGGTGGTTTGTGGAGAGGAACCTTGT |
| | GAAACCAGAAGGGAAAGTCATAGACCTCGGTTGTGGAAGAGGTGGCTGGTCATAT |
| | TATTGCGCTGGGCTGAAGAAAGTCACAGAAGTGAAAGGATACACGAAAGGAGGAC |
| | CTGGACATGAGGAACCAATCCCAATGGCAACCTATGGATGGAACCTAGTAAAGCT |
| | ATACTCCGGGAAAGATGTATTCTTTACACCACCTGAGAAATGTGACACCCTCTTGT |
| | GTGATATTGGTGAGTCCTCTCCGAACCCAACTATAGAAGAAGGAAGAACGTTACGT |
| | GTTCTAAAGATGGTGGAACCATGGCTCAGAGGAAACCAATTTTGCATAAAAATTCT |
| | AAATCCCTATATGCCGAGTGTGGTAGAAACTTTGGAGCAAATGCAAAGAAAACAT |
| | GGAGGAATGCTAGTGCGAAATCCACTCTCAAGAAACTCCACTCATGAAATGTACTG |
| | GGTTTCATGTGGAACAGGAAACATTGTGTCAGCAGTAAACATGACATCTAGAATGC |
| | TGCTAAATCGATTCACAATGGCTCACAGGAAGCCAACATATGAAAGAGACGTGGA |
| | CTTAGGCGCTGGAACAAGACATGTGGCAGTAGAACCAGAGGTGGCCAACCTAGAT |
| | ATCATTGGCCAGAGGATAGAGAATATAAAAAATGAACACAAATCAACATGGCATT |
| | ATGATGAGGACAATCCATACAAAACATGGGCCTATCATGGATCATATGAGGTCAA |
| | GCCATCAGGATCAGCCTCATCCATGGTCAATGGTGTGGTGAGACTGCTAACCAAAC |
| | CATGGGATGTCATTCCCATGGTCACACAAATAGCCATGACTGACACCACACCCTTT |
| | GGACAACAGAGGGTGTTTAAAGAGAAAGTTGACACGCGTACACCAAAAGCGAAAC |
| | GAGGCACAGCACAAATTATGGAGGTGACAGCCAGGTGGTTATGGGGTTTTCTCTCT |
| | AGAAACAAAAAACCCAGAATCTGCACAAGAGAGGAGTTCACAAGAAAAGTCAGGT |
| | CAAACGCAGCTATTGGAGCAGTGTTCGTTGATGAAAATCAATGGAACTCAGCAAA |
| | AGAGGCAGTGGAAGATGAACGGTTCTGGGACCTTGTGCACAGAGAGAGGGAGCTT |
| | CATAAACAAGGAAAATGTGCCACGTGTGTCTACAACATGATGGGAAAGAGAGAGA |
| | AAAAATTAGGAGAGTTCGGAAAGGCAAAAGGAAGTCGCGCAATATGGTACATGTG |
| | GTTGGGAGCGCGCTTTTTAGAGTTTGAAGCCCTTGGTTTCATGAATGAAGATCACT |
| | GGTTCAGCAGAGAGAATTCACTCAGTGGAGTGGAAGGAGAAGGACTCCACAAACT |
| | TGGATACATACTCAGAGACATATCAAAGATTCCAGGGGGAAATATGTATGCAGAT |
| | GACACAGCCGGATGGGACACAAGAATAACAGAGGATGATCTTCAGAATGAGGCCA |
| | AAATCACTGACATCATGGAACCTGAACATGCCCTATTGGCCACGTCAATCTTTAAG |
| | CTAACCTACCAAAACAAGGTAGTAAGGGTGCAGAGACCAGCGAAAAATGGAACCG |
| | TGATGGATGTCATATCCAGACGTGACCAGAGAGGAAGTGGACAGGTTGGAACCTA |
| | TGGCTTAAACACCTTCACCAACATGGAGGCCCAACTAATAAGACAAATGGAGTCTG |
| | AGGGAATCTTTTCACCCAGCGAATTGGAAACCCCAAATCTAGCCGAAAGAGTCCTC |
| | GACTGGITGAAAAAACATGGCACCGAGAGGCTGAAAAGAATGGCAATCAGTGGAG |
| | ATGACTGTGTGGTGAAACCAATCGATGACAGATTTGCAACAGCCTTAACAGCTTTG |
| | AATGACATGGGAAAGGTAAGAAAAGACATACCGCAATGGGAACCTTCAAAAGGAT |
| | GGAATGATTGGCAACAAGTGCCTTTCTGTTCACACCATTTCCACCAGCTGATTATGA |
| | AGGATGGGAGGGAGATAGTGGTGCCATGCCGCAACCAAGATGAACTTGTAGGTAG |
| | GGCCAGAGTATCACAAGGCGCCGGATGGAGCTTGAGAGAAACTGCATGCCTAGGC |
| | AAGTCATATGCACAAATGTGGCAGCTGATGTACTTCCACAGGAGAGACTTGAGATT |
| | AGCGGCTAATGCTATCTGTTCAGCCGTTCCAGTTGATTGGGTCCCAACCAGCCGCA |
| | CCACCTGGTCGATCCATGCCCACCATCAATGGATGACAACAGAAGACATGTTGTCA |
| | GTGTGGAATAGGGTTTGGATAGAGGAAAACCCATGGATGGAGGACAAGACTCATG |
| | TGTCCAGTTGGGAAGACGTTCCATACCTAGGAAAAAGGGAAGATCAATGGTGTGG |
| | TTCCCTAATAGGCTTAACAGCACGAGCCACCTGGGCCACCAACATACAAGTGGCCA |
| | TAAACCAAGTGAGAAGGCTCATTGGGAATGAGAATTATCTAGACTTCATGACATCA |
| | ATGAAGAGATTCAAAAACGAGAGTGATCCCGAAGGGGCACTCTGGTAAGCCAACT |
| | CATTCACAAAATAAAGGAAAATAAAAAATCAAACAAGGCAAGAAGTCAGGCCGG |
| | ATTAAGCCATAGCACGGTAAGAGCTATGCTGCCTGTGAGCCCCGTCCAAGGACGTA |
| | AAATGAAGTCAGGCCGAAAGCCACGGTTCGAGCAAGCCGTGCTGCCTGTAGCTCC |
| | ATCGTGGGGATGTAAAAACCCGGGAGGCTGCAAACCATGGAAGCTGTACGCATGG |
| | GGTAGCAGACTAGTGGTTAGAGGAGACCCCTCCCAAGACACAACGCAGCAGCGGG |
| | GCCCAACACCAGGGGAAGCTGTACCCTGGTGGTAAGGACTAGAGGTTAGAGGAGA |
| | CCCCCCGCACAACAACAAACAGCATATTGACGCTGGGAGAGACCAGAGATCCTGC |
| | TGTCTCTACAGCATCATTCCAGGCACAGAACGCCAAAAAATGGAATGGTGCTGTTG |
| | AATCAACAGGTTCT |
| 9 | MNNQRKKTGRPSFNMLKRARNRVSTVSQLAKRFSKGLLSGQGPMKLVMAFIAFLRFL |
| | AIPPTAGILARWGSFKKNGAIKVLRGFKKEISNMLNIMNRRKRSVTMLLMLLPTALAFH |
| | LTTRGGEPHMIVSKQERGKSLLFKTSAGVNMCTLIAMDLGELCEDTMTYKCPRITETEP |
| | DDVDCWCNATETWVTYGTCSQTGEHRRDKRSVALAPHVGLGLETRTETWMSSEGAW |
| | KQIQKVETWALRHPGFTVIALFLAHAIGTSITQKGIIFILLMLVTPSMAMRCVGIGNRDF |
| | VEGLSGATWVDVVLEHGSCVTTMAKDKPTLDIELLKTEVTNPAVLRKLCIEAKISNTTT |
| | DSRCPTQGEATLVEEQDTNFVCRRTFVDRGWGNGCGLFGKGSLITCAKFKCVTKLEGK |
| | IVQYENLKYSVIVTVHTGDQHQVGNETTEHGTTATITPQAPTSEIQLTDYGALTLDCSP |
| | RTGLDFNEMVLLTMKKKSWLVHKQWFLDLPLPWTSGASTSQETWNRQDLLVTFKTA |
| | HAKKQEVVVLGSQEGAMHTALTGATEIQTSGTTTIFAGHLKCRLKMDKLILKGMSYV |
| | MCTGSFKLEKEVAETQHGTVLVQVKYEGTDAPCKIPFSSQDEKGVTQNGRLITANPIVT |
| | DKEKPVNIEAEPPFGESYIVVGAGEKALKLSWFKKGSSIGKMFEATARGARRMAILGD |
| | TAWDFGSIGGVFTSVGKLIHQIFGTAYGVLFSGVSWTMKIGIGILLTWLGLNSRSTSLSM |
| | TCIAVGMVTLYLGVMVQADSGCVINWKGRELKCGSGIFVTNEVHTWTEQYKFQADSP |
| | KRLSAAIGKAWEEGVCGIRSATRLENIMWKQISNELNHILLENDMKFTVVVGDVSGIL |
| | AQGKKMIRPQPMEHKYSWKSWGKAKIIGADVQNTTFIIDGPNTPECPDNQRAWNIWE |
| | VEDYGFGIFTTNIWLKLRDSYTQVCDHRLMSAAIKDSKAVHADMGYWIESEKNETWK |
| | LARASFIEVKTCIWPKSHTLWSNGVLESEMIIPKIYGGPISQHNYRPGYFTQTAGPWHLG |
| | KLELDFDLCEGTTVVVDEHCGNRGPSLRTTTVTGKTIHEWCCRSCTLPPLRFKGEDGC |
| | WYGMEIRPVKEKEENLVKSMVSAGSGEVDSFSLGLLCISIMIEEVMRSRWSRKMLMTG |
| | TLAVFLLLTMGQLTWNDLIRLCIMVGANASDKMGMGTTYLALMATFRMRPMFAVGL |
| | LFRRLTSREVLLLTVGLSLVASVELPNSLEELGDGLAMGIMMLKLLTDFQSHQLWATL |
| | LSLTFVKTTFSLHYAWKTMAMILSIVSLFPLCLSTTSQKTTWLPVLLGSLGCKPLTMFLI |
| | TENKIWGRKSWPLNEGIMAVGIVSILLSSLLKNDVPLAGPLIAGGMLIACYVISGSSADL |
| | SLEKAAEVSWEEEAEHSGASHNILVEVQDDGTMKIKDEERDDTLTILLKATLLAISGVY |
| | PMSIPATLFVWYFWQKKKQRSGVLWDTPSPPEVERAVLDDGIYRILQRGLLGRSQVGV |
| | GVFQEGVFHTMWHVTRGAVLMYQGKRLEPSWASVKKDLISYGGGWRFQGSWNAGE |
| | EVQVIAVEPGKNPKNVQTAPGTFKTPEGEVGAIALDFKPGTSGSPIVNREGKIVGLYGN |
| | GVVTTSGTYVSAIAQAKASQEGPLPEIEDEVFRKRNLTIMDLHPGSGKTRRYLPAIVRE |
| | AIKRKLRTLVLAPTRVVASEMAEALKGMPIRYQTTAVKSEHTGKEIVDLMCHATFTMR |
| | LLSPVRVPNYNMIIMDEAHFTDPASIAARGYISTRVGMGEAAAIFMTATPPGSVEAFPQ |
| | SNAVIQDEERDIPERSWNSGYDWITDFPGKTVWFVPSIKSGNDIANCLRKNGKRVVQLS |
| | RKTFDTEYQKTKNNDWDYVVTTDISEMGANFRADRVIDPRRCLKPVILKDGPERVILA |
| | GPMPVTVASAAQRRGRIGRNQNKEGDQYIYMGQPLNNDEDHAHWTEAKMLLDNINT |
| | PEGIIPALFEPEREKSAAIDGEYRLRGEARKTFVELMRRGDLPVWLSYKVASEGFQYSD |
| | RRWCFDGERNNQVLEENMDVEIWTKEGERKKLRPRWLDARTYSDPLALREFKEFAAG |
| | RRSVSGDLILEIGKLPQHLTQRAQNALDNLVMLHNSEQGGKAYRHAMEELPDTIETLM |
| | LLALIAVLTGGVTLFFLSGRGLGKTSIGLLCVIASSALLWMASVEPHWIAASIILEFFLM |
| | VLLIPEPDRQRTPQDNQLAYVVIGLLFMILTVAANEMGLLETTKKDLGIGHAAAENHH |
| | HAAMLDVDLHPASAWTLYAVATTIITPMMRHTIENTTANISLTAIANQAAILMGLDKG |
| | WPISKMDIGVPLLALGCYSQVNPLTLTAAVLMLVAHYAIIGPGLQAKATREAQKRTAA |
| | GIMKNPTVDGIVAIDLDPVVYDAKFEKQLGQIMLLILCTSQILLMRTTWALCESITLATG |
| | PLTTLWEGSPGKFWNTTIAVSMANIFRGSYLAGAGLAFSLMKSLGGGRRGTGAQGETL |
| | GEKWKRQLNQLSKSEFNTYKRSGIIEVDRSEAKEGLKRGETTKHAVSRGTAKLRWFVE |
| | RNLVKPEGKVIDLGCGRGGWSYYCAGLKKVTEVKGYTKGGPGHEEPIPMATYGWNL |
| | VKLYSGKDVFFTPPEKCDTLLCDIGESSPNPTIEEGRTLRVLKMVEPWLRGNQFCIKILN |
| | PYMPSVVETLEQMQRKHGGMLVRNPLSRNSTHEMYWVSCGTGNIVSAVNMTSRMLL |
| | NRFTMAHRKPTYERDVDLGAGTRHVAVEPEVANLDIIGQRIENIKNEHKSTWHYDEDN |
| | PYKTWAYHGSYEVKPSGSASSMVNGVVRLLTKPWDVIPMVTQIAMTDTTPFGQQRVF |
| | KEKVDTRTPKAKRGTAQIMEVTARWLWGFLSRNKKPRICTREEFTRKVRSNAAIGAVF |
| | VDENQWNSAKEAVEDERFWDLVHRERELHKQGKCATCVYNMMGKREKKLGEFGKA |
| | KGSRAIWYMWLGARFLEFEALGFMNEDHWFSRENSLSGVEGEGLHKLGYILRDISKIP |
| | GGNMYADDTAGWDTRITEDDLQNEAKITDIMEPEHALLATSIFKLTYQNKVVRVQRPA |
| | KNGTVMDVISRRDQRGSGQVGTYGLNTFTNMEAQLIRQMESEGIFSPSELETPNLAERV |
| | LDWLKKHGTERLKRMAISGDDCVVKPIDDRFATALTALNDMGKVRKDIPQWEPSKG |
| | WNDWQQVPFCSHHFHQLIMKDGREIVVPCRNQDELVGRARVSQGAGWSLRETACLG |
| | KSYAQMWQLMYFHRRDLRLAANAICSAVPVDWVPTSRTTWSIHAHHQWMTTEDMLS |
| | VWNRVWIEENPWMEDKTHVSSWEDVPYLGKREDQWCGSLIGLTARATWATNIQVAI |
| | NQVRRLIGNENYLDFMTSMKRFKNESDPEGALW |

In some instances, the DV is serotype 2. In some instances the DV serotype 2 is DENV-2 strain #1710. DENV-2 strain #1710 is from a sample taken from Puerto Rico in 1985 and characterized as type A from a restriction site specific RT-PCR analysis using 4 primers (see Table 3) specific to the envelope gene region. See Harris et al., Virology 253, 86-95 (1999). Restriction site specific RT-PCR with these primers produces amplification products of 582 base pairs, 754 base pairs, and possibly 676 base pairs. The DENV-2 strain #1710 is recorded in a CDC database as entry number 555. See Harris (1999). The DENV-2 strain #1710 was isolated during a Puerto Rican epidemic. This outbreak had 9,540 suspected cases of DV, with one suspected, but no confirmed deaths due to the virus, which indicates the toxicity of DENV-2 strain #1710 is very low and therefore suitable for the methods disclosed herein.

TABLE 3
Sequence and Position of Primers to Amplify DENV-2
viruses

| | | Genome | |
|---|---|---|---|
| Primer | Sequence | Position | Strand |
| RSS1 | 5′-GGATCCCAAGAAGGGGCCAT-3′ | 1696-1715 | + |
| | (SEQ ID NO: 3) | | |
| RSS2 | 5′-GGCAGCTCCATAGATTGCT-3′ | 2277-2259 | − |
| | (SEQ ID NO: 4) | | |
| RSS3 | 5′-GGTGTTGCTGCAGATGGAA-3′ | 1524-1542 | + |
| | (SEQ ID NO: 5) | | |
| RSS4 | 5′-GTGTCACAGACAGTGAGGT-3′ | 2371-2353 | − |
| | (SEQ ID NO: 6) | | |

Advantageous DV characteristics for use as a potent immune-stimulant in cancer immunotherapies are described herein. DV has affinity for immature B-lymphocytes and antigen-presenting cells (APC) of monocyte/macrophage and dendritic cell (DC) lineage. A unique feature of DV is that primary infections result in activation of a T_{H}1-type response of CD4+ and CD8+ helper-inducer and cytotoxic-effector CTL. By infecting, but not killing the APC, DV up-regulates their CD80 and CD83 expression, resulting in a pro-inflammatory T_{H}1 cytokine profile. Primary DV infections induce a T_{H}1 type response with activated CD4⁺ and CD8⁺ effector T cells as well as LAK cells. This type of response is seen in patients having complete responses to cancer immunotherapies (see Table 4).

TABLE 4
Tumor immune evasion mechanisms and DV infection

| Immune evasion | Dengue counter-attack |
|---|---|
| Low levels of MHC on | High Interferon-γ raises MHC levels by |
| tumor cell prevent | up-regulating MHC gene expression |
| CTL recognition | |
| Point mutations in | LAK/CIK cells target “escaped” tumor |
| Tumor Peptides prevent | cells expressing aberrant peptides or MHC |
| TCR binding | |
| Tumor vessels lack factors | Hi [TNF-α] restores gaps by altering |
| for CTL attachment and | PECAM-1, restores ICAM-1/VCAM-1 |
| trafficking | expression and P and E-selectins |
| FasL can kill Fas⁺ | Hi [IL-6, 15] protects Fas⁺ CTL |
| CTL by triggering | by up-regulating FLIP ligand |
| apoptosis | |
| HLA-G protects from | Hi [IL-2, 7, 12, 15] raise activation of NK |
| NK Cells | |
| Stromal barriers | Hi [IFN-γ] activates Macrophages to M₁ |
| inhibit CTL | |
| Myeloid-Derived | iNKT Cells can decrease MDSC |
| Suppressor Cells, | |
| (MDSC) | |
| CTL inactivated | T_{H}1 cytokines reactivate tolerant CTL |
| by TGF-β | |
| Tumor PI-9 blocks | Hi [CD8] & ICAM-1 expression can restore |
| CTL killing | low-avidity CTL recognition and lysis by |
| | stabilizing weak interactions between TCR |
| | and MHC + self-peptide |
| T-regulatory | Hi CD4^{Helper} cells overcome CD4^{Reg} cells |
| cells block CTL | |

In primary infections, the death rate from DV is very low (1 in 61,000 per Manson's Tropical Diseases). The virus infects but does not kill APC of the monocyte-macrophage and Dendritic Cell lineage. These infected APC then begin a cytokine cascade of the pro-inflammatory (TNF-alpha and IL-1 beta), and TH1 (IL-2, IL-7, IL-12, IL-15, and IL-21) types. These cytokines result in strong activation of both the adaptive (CTL) and innate (NK) immune systems. After a 3-5 day incubation period, the fever rises to 39.5-40.5° C., and remains elevated for 4-5 days. The patient experiences intense headache, joint pain, malaise, and sensitivity to light. A rash covering the chest back and sometimes legs and arms develops by day 3 of fever. Clinically, dengue infections result in lowered platelet counts leading to hemorrhage, which ranges from minor to life-threatening in case of shock syndrome. With proper supportive care based on judicious fluid management, recovery is complete in 99% of cases.

Provided herein are compositions and methods for reducing the cancer cells in a subject in need thereof comprising administering a Dengue virus, wherein the method provides for reduction of cancer cells in the subject by at least about 40%. In some instances, the methods and compositions disclosed herein provide for reduction of cancer cells in the subject by at least about 40%, about 45%, about 50%, about 55%, about 60%, about 65%, about 70%, about 75%, about 80%, about 85%, about 90%, about 95%, or about 100%.

### Pharmaceutical Compositions

Provided herein are compositions comprising an effective amount of Dengue virus (DV) to reduce cancer cells in a subject in need thereof. In some instances, the effective amount is about 10⁵ plaque-forming units (PFU). In some instances, the effective amount of DV is about 10,000 to about 90,000 PFU; about 20,000 to about 60,000 PFU; about 50,000 to about 80,000 PFU. In some instances, the effective amount of DV is greater than about 40,000 PFU or greater than about 30,000 PFU. In some instances, the effective amount of DV is less than about 90,000 PFU; less than about 30,000 PFU; or less than about 20,000 PFU. The DV may be a strain described in Table 1.

Provided herein are compositions comprising an effective amount of Dengue virus sufficient to increase a level of at least one cytokine in the subject. In some instances, the effective amount is an amount sufficient to increase a level of at least one cytokine in the blood of the subject. In some instances, the effective amount is an amount sufficient to increase a level of at least one cytokine in a serum sample of the subject. In some instances, the effective amount is an amount sufficient to significantly increase the level of the at least one cytokine. In some instances, the effective amount is an amount sufficient to increase the level of the at least one cytokine by about 2% to about 20,000%. In some instances, the effective amount is an amount sufficient to increase the level of the at least one cytokine by about 50% to about 20,000%. In some instances, the effective amount is an amount sufficient to increase the level of the at least one cytokine by about 100% to about 20,000%. In some instances, the effective amount is an amount sufficient to increase the level of the at least one cytokine by about 100% to about 15,000%. In some instances, the effective amount is an amount sufficient to increase the level of the at least one cytokine by about 100% to about 14,000%. In some instances, the effective amount is an amount sufficient to increase the level of the at least one cytokine by about 50% to about 15,000%. In some instances, the effective amount is an amount sufficient to increase the level of the at least one cytokine by about 50% to about 14,000%.

Provided herein are compositions comprising an amount of Dengue virus sufficient to increase a level of at least one cytokine in the subject. In some instances, the at least one cytokine is an interleukin (IL). In some instances, the at least one cytokine is an interferon (IFN). In some instances, the at least one cytokine is an interleukin. In some instances, the at least one cytokine is selected from tumor necrosis factor (TNF) alpha, IFN alpha, IFN beta, IFN gamma, interferon gamma induced protein 10 (IP-10), IL-12, IL-2R, IL-7, IL-15, granulocyte macrophage colony stimulating factor (GM-CSF), and a combination thereof. In some instances the level of TNF alpha is increased from about 50% to about 500%. In some instances the level of TNF alpha is increased from about 50% to about 300%. In some instances the level of TNF alpha is increased from about 50% to about 240%. In some instances the level of IFN alpha is increased from about 50% to about 800%. In some instances the level of IFN alpha is increased from about 50% to about 500%. In some instances the level of IFN alpha is increased from about 50% to about 420%. In some instances the level of IFN beta is increased from about 50% to about 20,000%. In some instances the level of IFN beta is increased from about 50% to about 14,000%. In some instances the level of IFN gamma is increased from about 50% to about 200%. In some instances the level of IFN gamma is increased from about 50% to about 100%. In some instances the level of IP-10 is increased from about 50% to about 8000%. In some instances the level of IP-10 is increased from about 50% to about 5000%. In some instances the level of IP-10 is increased from about 50% to about 4000%. In some instances the level of IL-12 is increased from about 20% to about 200%. In some instances the level of IL-12 is increased from about 20% to about 100%. In some instances the level of IL-12 is increased from about 20% to about 80%. In some instances the level of IL-15 is increased from about 20% to about 200%. In some instances the level of IL-15 is increased from about 20% to about 200%. In some instances the level of IL-15 is increased from about 20% to about 100%. In some instances the level of IL-7 is increased from about 50% to about 1000%. In some instances the level of IL-7 is increased from about 50% to about 1000%. In some instances the level of IL-7 is increased from about 50% to about 500%. In some instances the level of GM-CSF is increased from about 50% to about 1000%. In some instances the level of GM-CSF is increased from about 50% to about 400%. In some instances the level of GM-CSF is increased from about 50% to about 350%. In some instances the level of IL-12R is increased from about 20% to about 200%. In some instances the level of IL-12R is increased from about 20% to about 150%.

Provided herein are compositions comprising an effective amount of Dengue virus (DV), wherein the effective amount is an amount sufficient to increase expression of a protein in tumor cell. In some instances, the effective amount is an amount sufficient to increase expression of a protein expressed on a tumor cell. In some instances, the protein is a checkpoint protein. In some instances, this makes the tumor cell a better target for checkpoint inhibitors. In some instances, the checkpoint protein is programmed death-ligand 1 (PD-L1). In some instances, the effective amount increases the expression of PD-L1 by about 10% to about 100%. In some instances, the effective amount increases the expression of PD-L1 by about 10% to about 20%. In some instances, the effective amount is an amount sufficient to increase expression of a complex of proteins expressed on a tumor cell. In some instances, the complex is a major histocompatibility complex (MHC). In some instances, the MHC is a Class I MHC. In some instances, the effective amount increases the expression of the MHC by about 10% to about 60%. In some instances, the effective amount increases the expression of the MHC by about 10% to about 100%. In some instances, the effective amount increases the expression of the MHC by about 10% to about 150%.

Provided herein are compositions comprising an effective amount of Dengue virus (DV) to reduce cancer cells in a subject in need thereof, wherein the effective amount is an amount sufficient to increase expression of a protein on an immune cell of the subject. In some instances, the effective amount is an amount sufficient to increase expression of a protein in the immune cell. In some instances, the immune cell is a T cell. In some instances, the protein is intercellular adhesion molecule (e.g., joins two cells together). In some instances, the intercellular adhesion molecule is intercellular adhesion molecule 1 (ICAM-1). In some instances, the effective amount increases the expression of ICAM-1 by about 10% to about 500%. In some instances, the effective amount increases the expression of ICAM-1 by about 10% to about 300%. Provided herein are compositions comprising an effective amount of Dengue virus. In some instances, compositions disclosed herein comprise a sugar. In some instances, compositions disclosed herein comprise a surfactant. In some instances, compositions disclosed herein comprise a protein. In some instances, compositions disclosed herein comprise a salt. In some instances, compositions disclosed herein comprise a non-ionic surfactant, a non-reducing sugar, a salt, a carrier protein, or a combination thereof.

Provided herein are compositions comprising an effective amount of Dengue virus to reduce cancer cells in a subject in need thereof. In some instances, the composition comprises a non-ionic surfactant. In some instances, the non-ionic surfactant is a non-ionic detergent. In some instances, the non-ionic surfactant is an agent comprising a hydrophobic chain. In some instances, the non-ionic surfactant is an agent comprising polyoxyethylene. In some instances, the non-ionic surfactant is an agent comprising polyoxypropylene. In some instances, the non-ionic surfactant is an agent comprising a polyoxyethylene-polyoxypropylene block copolymer. In some instances, the non-ionic surfactant is an agent that acts as a stabilizer of a cell membrane. In some instances, the non-ionic surfactant is an agent that protects from cell membrane shearing. In some instances, the non-ionic surfactant is an agent that acts as an anti-foaming agent. In some instances, the non-ionic surfactant comprises pluronic F-68. In some instances, the non-ionic surfactant consists essentially of pluronic F-68. Additional non-limiting examples of non-ionic surfactants contemplated for use in the compositions disclosed herein include alkyl polyglycoside, cetomacrogol 1000, cetostearyl alcohol, cetyl alcohol, cocamide DEA, cocamide MEA, decyl glucoside, decyl polyglucose, glycerol monostearate, IGEPAL CA-630, isoceteth-20, lauryl glucoside, maltosides, monolaurin, mycosubtilin, narrow-range ethoxylate, nonidet P-40, nonoxynol-9, nonoxynols, NP-40, octaethylene glycol monododecyl ether, N-octyl beta-d-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80, and combinations thereof. In some instances, the non-ionic surfactant is present in the composition at a concentration of about 0.01% w/v to about 10% w/v. In some instances, the non-ionic surfactant is present in the composition at a concentration of about 0.1% w/v to about 5% w/v. In some instances, the non-ionic surfactant is present in the composition at a concentration of about 1% w/v to about 5% w/v. In some instances, the non-ionic surfactant is present in the composition at a concentration of about 2% w/v.

Provided herein are compositions comprising an amount of Dengue virus sufficient to reduce cancer cells in a subject in need thereof and a non-reducing sugar. In some instances, the non-reducing sugar is a sugar capable of trapping water molecules. In some instances, the non-reducing sugar acts as a cryoprotectant, protecting the viability of the Dengue virus during freezing and thawing. In some instances, the non-reducing sugar comprises a disaccharide. In some instances, the non-reducing sugar comprises an alpha, alpha-1, 1-glucoside bond between two alpha glucose units. In some instances, the non-reducing sugar consists essentially of a disaccharide. In some instances, the non-reducing sugar comprises a trehalose. Trehalose is also known as a-D-glucopyranosyl-(1→1)-a-D-glucopyranoside, mycose, and tremalose. In some embodiments, the non-reducing sugar consists essentially of a trehalose. In some instances, the trehalose is alpha-trehalose. In some instances, the trehalose is D-(+)-Trehalose dehydrate. In some instances, the trehalose has the chemical formula of C₁₂H₂₂O₁₁.2H₂O. In some instances, the non-reducing sugar is present in the composition at a concentration of about 5% w/v to about 25% w/v. In some instances, the non-reducing sugar is present in the composition at a concentration of about 1% w/v to about 10% w/v. In some instances, the non-reducing sugar is present in the composition at a concentration of about 10% w/v to about 20% w/v. In some instances, the non-reducing sugar is present in the composition at a concentration of about 15% w/v.

Provided herein are compositions comprising an effective amount of Dengue virus to reduce cancer cells in a subject in need thereof, and a carrier protein. Carrier proteins may function as a carrier or stabilizer for steroids, fatty acids, or hormones. In some instances, the carrier protein is a protein capable of stabilizing a virus envelope in storage conditions (e.g., below room temperature). In some instances, the carrier protein is a soluble monomeric protein. In some instances, the carrier protein is albumin. In some instances, the carrier protein is a human protein ensuring compositions disclosed herein are compliant with good manufacturing protocol (GMP) standard. In some instances the carrier protein is human albumin. In some instances, the carrier protein is present in the composition at a concentration of about 0.1% w/v to about 10% w/v. In some instances, the carrier protein is present in the composition at a concentration of about 1% w/v to about 5% w/v. In some instances, the carrier protein is present in the composition at a concentration of about 2% w/v.

Provided herein are compositions comprising an effective amount of Dengue virus to reduce cancer cells in a subject in need thereof. In some instances, the composition comprises a salt. In some instances, the salt comprises calcium, magnesium, potassium, sodium, boron. In some instances, the salt is a phosphate salt, a chloride salt, a sulfate salt or a dichromate salt. In some instances, the salt is calcium chloride. In some instances, the salt is magnesium chloride. In some instances, compositions comprise calcium chloride and magnesium chloride. In some instances, the salt is present in the composition at a concentration of about 0.1 mM to about 10 mM. In some instances, the salt is present in the composition at a concentration of about 0.1 mM to about 5 mM. In some instances, the salt is present in the composition at a concentration of about 0.1 mM to about 2 mM. In some instances, the salt is present in the composition at a concentration of about 1 mM. In some instances, compositions comprise calcium chloride and magnesium chloride wherein calcium chloride is present in the composition at about 0.1 mM to about 10 mM, and magnesium chloride is present in the composition at about 0.1 mM to about 10 mM. In some instances, compositions comprise calcium chloride and magnesium chloride wherein calcium chloride is present in the composition at about 1 mM, and magnesium chloride is present in the composition at about 1 mM.

In some instances, compositions and methods disclosed herein modify expression of genes in cells of a subject. Exemplary modification of gene expression may be increased or decreased expression. Expression of genes in cells of the subject may be increased by DV infection, including, but not limited to, IL-1 beta, IL-2, IL-7, IL-12, IL-15, IFN-alpha, IFN-gamma, TNF-alpha, TNF-beta, GM-CSF, CD8 antigen, ICOSLG, CCL3, CCL5, TRAIL, IP10, GNLY, GZMA, HLA-DRA, HLA-DP alphal, HLA-DP beta 1, and ZAP70. Increased levels of proteins corresponding to these genes may be observed in circulating fluids of the subject. Levels may be increased at least 2-fold. Levels may be increased between 2-fold and 1000-fold. Levels may be increased between 2-fold and 100-fold. Levels may be increased between 2-fold and 10-fold. Cell types of a subject administered DV may be increased by DV infection, including, but not limited to, CD8+CD44+62L− cells, CD4+CD44+CD62L10 cells, HLA-DR+CD8+ cells, Tia-1 CD8+ cells, VLA-4 CD8+ cells, ICAM-1 CD8+ cells, and LFA-1 CD8+ cells. In some instances, TNF-α, is released by the immune system during DV infection. TNFα is an inflammatory cytokine with pleiotropic effects, including direct killing of tumor cells via TRAIL (TNF-Apoptosis-Inducing-Ligand).

In some instances, DV induces high levels of soluble TRAIL (sTRAIL) from a variety of cells including γδCTL, activated M1 macrophages and plasmacytoid DC (pDC). In some instances, DV activates IFNβ, a multifunctional cytokine with a 10-fold higher affinity for the same receptor as IFNα. IFNβ has similar antiviral properties in suppressing transcription of viral RNA, but is much more potent than IFNα in inducing apoptosis in tumor cells. Nitric oxide and IFNβ could act in a synergistic fashion during dengue infection. These molecules may work in tandem to overcome resistance to apoptosis mediated by the high levels of sTRAIL induced by M₁ macrophages, pDC, and δγ CTL.

Provided herein are pharmaceutical compositions comprising which may optionally comprise one strain of Dengue virus. In some cases, from about 1, 2, 3, 4, 5, or more strains of Dengue Virus may be utilized as part of a method or composition described herein. In some instances, the pharmaceutical compositions comprise at least a portion of a Dengue virus. The portion of the Dengue virus may be a portion sufficient to generate an immune response in a subject receiving the pharmaceutical composition. The compositions may further comprise one or more pharmaceutically acceptable salts, excipients or vehicles. Pharmaceutically acceptable salts, excipients, or vehicles for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, co-solvents, wetting agents, complexing agents, buffering agents, antimicrobials, and surfactants.

In some instances, the carriers disclosed herein comprise neutral buffered saline. The pharmaceutical compositions may include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also may be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers may be conventional buffers such as acetate, borate, citrate, phosphate, bicarbonate, or Tris-HCl. Acetate buffer may be about pH 4-5.5, and Tris buffer may be about pH 7-8.5.

Provided herein are compositions that comprise a Dengue virus, wherein the composition is in liquid form, lyophilized form or freeze-dried form and may include one or more lyoprotectants, excipients, surfactants, high molecular weight structural additives and/or bulking agents. In some instances, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the virus upon lyophilization. Exemplary lyoprotectant concentrations for sugars (e.g., sucrose, lactose, trehalose) in the pre-lyophilized formulation are from about 10 mM to about 400 mM.

Provided herein are compositions that comprise a Dengue virus disclosed herein, wherein the compositions are suitable for injection or infusion. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

Devices for injection of a Dengue Virus described herein may be configured for subcutaneous injection. In some instances, the device is not configured for intradermal injection. The device may have a needle gauge size of 30 to 19 G on an ISO scale. The device may have a needle gauge size of 27 to 19 G on an ISO scale. The device may have a needle gauge size of 24 to 19 G on an ISO scale. The device may have a needle gauge size of 23 to 19 G on an ISO scale. The device may have a needle gauge size of 22 to 19 G on an ISO scale. The device may have a needle gauge size of 21 to 19 G on an ISO scale. The device may have a needle length of ⅜ inches to ¾ inches. The device may have a needle length of ½ inches to ⅝ inches. The needle may be injected at an angle of 45 degrees to 90 degrees for subcutaneous injection. The injection site may be in the deltoid muscle of arm, or vastus lateralis muscle of thigh.

Disclosed herein, are methods of manufacturing and storing the DV. In some instances, the DV is stored in a 0.5 ml container. In some instances, the DV is stored in a 1.0 ml container. In some instances, the DV is stored in a 1.5 ml container. In some instances, the DV is stored in a 2.0 ml container. In some instances, the DV is stored in a 2.5 ml container. In some instances, the DV is stored in a 3.0 ml container. In some instances, the DV is stored in a 3.5 ml container. In some instances, the DV is stored in a 4.0 ml container. In some instances, the DV is stored in a 4.5 ml container. In some instances, the DV is stored in a 5.0 ml container. In some instances, the DV is stored in a 5.5 ml container. In some instances, the DV is stored in a 6.0 ml container. In some instances, the DV is stored in a 6.5 ml container. In some instances, the DV is stored in a 7.0 ml container. In some instances, the DV is stored in a 7.5 ml container. In some instances, the DV is stored in an 8.0 ml container. In some instances, the DV is stored in an 8.5 ml container. In some instances, the DV is stored in a 9.0 ml container. In some instances, the DV is stored in a 9.5 ml container. In some instances, the DV is stored in a 10 ml container. Exemplary containers include, without limitation, a bottle, vial, can, or syringe.

Provided herein are pharmaceutical compositions that comprise a Dengue virus disclosed herein, and a non-aqueous solvent. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, antioxidants, chelating agents, inert gases and the like.

Provided herein are pharmaceutical compositions that comprise a Dengue virus disclosed herein, wherein the pharmaceutical composition is formulated for inhalation, such as for example, as a dry powder. Suitable and/or preferred pharmaceutical formulations may be determined in view of the present disclosure and general knowledge of formulation technology, depending upon the intended route of administration, delivery format, and desired dosage. Regardless of the manner of administration, an effective dose may be calculated according to patient body weight, body surface area, or organ size. Further refinement of the calculations for determining the appropriate dosage for treatment involving each of the formulations described herein are routinely made in the art and is within the ambit of tasks routinely performed in the art. Appropriate dosages may be ascertained through use of appropriate dose-response data.

### Methods of Administration

Provided herein are methods comprising administering Dengue virus to a subject in need thereof. In some instances, the virus is provided in an aqueous form. In some instances, the virus is lyophilized and reconstituted in an aqueous solution (e.g., saline solution). In some instances, the virus is administered by a route selected from subcutaneous injection, intramuscular injection, intradermal injection, percutaneous administration, intravenous (“i.v.”) administration, intranasal administration, intralymphatic injection, and oral administration. In some instances, the subject is infused with the virus by an intralymphatic microcatheter.

In some instances, the methods disclosed herein comprise administering Dengue virus at a dose of about 0.5 ml of 10⁶ pfu/ml. In some instances, the dose is between about 10³ pfu/ml and about 10⁸ pfu/ml. In some instances, the dose is between about 10³ pfu/ml and about 10⁶ pfu/ml. In some instances, the dose is between about 10³ pfu/ml to about 10⁴ pfu/ml, between about 10⁴ pfu/ml to about 10⁶ pfu/ml, between about 10⁶ pfu/ml to about 10⁸ pfu/ml, or between about 10⁸ pfu/ml to about 10¹⁰ pfu/ml. In some instances, the dose is from about 10¹ pfu/ml, 10² pfu/ml, 10³ pfu/ml, 10⁴ pfu/ml, 10⁵ pfu/ml, 10⁶ pfu/ml, 10⁷ pfu/ml, 10⁸ pfu/ml, or up to about 10⁹ pfu/ml. In some instances, a dose described herein is in a volume of about 0.01, 0.02, 0.03, 0.04, 0.05, 0.1, 0.2 ml or 0.3 ml. In some instances, a dose is in a volume of about 0.01 ml to about 0.03 ml, about 0.01 ml to about 0.1 ml, 0.03 ml to about 0.05 ml, 0.05 ml to about 0.07 ml, 0.07 ml to about 0.09 ml, 0.1 ml to about 0.2 ml, 0.2 ml to about 0.4 ml, 0.4 ml to about 0.6 ml.

In some instances, the methods disclosed herein comprise administering Dengue virus at a dose of about 0.5 ml of 10⁶ pfu/ml per day. In some instances, the dose is between about 10³ pfu/ml/day and about 10⁸ pfu/ml/day. In some instances, the dose is between about 10³ pfu/ml/day and about 10⁶ pfu/ml/day. In some instances, the methods disclosed herein comprise administering Dengue virus at more than one dose of about 0.5 ml of 10⁶ pfu/ml per day. In some instances, methods comprise administering a dose between about 10³ pfu/ml and about 10⁸ pfu/ml more than once per day. In some instances, methods comprise administering a dose between about 10³ pfu/ml and about 10⁶ pfu/ml more than once per day. In some instances, methods comprise administering a dose between about 10³ pfu/ml and about 10⁸ pfu/ml one to five times per day. In some instances, methods comprise administering a dose between about 10³ pfu/ml and about 10⁶ pfu/ml one to five times per day. In some instances, methods comprise administering a dose between about 10³ pfu/ml and about 10⁸ pfu/ml one to three times per day. In some instances, methods comprise administering a dose between about 10³ pfu/ml and about 10⁶ pfu/ml one to three times per day.

Provided herein are methods comprising administering a composition comprising Dengue virus to a subject in need thereof. In some instances, the composition comprises a sugar. In some instances, the composition comprises a surfactant. In some instances, the composition comprises a protein. In some instances, the composition comprises a salt. In some instances, the composition comprises a non-ionic surfactant, a non-reducing sugar, a salt, a carrier protein, or a combination thereof. In some instances, the composition comprises a non-ionic surfactant. In some instances, the non-ionic surfactant is a non-ionic detergent. In some instances, the non-ionic surfactant is an agent comprising a hydrophobic chain. In some instances, the non-ionic surfactant is an agent comprising polyoxyethylene. In some instances, the non-ionic surfactant is an agent comprising polyoxypropylene. In some instances, the non-ionic surfactant is an agent comprising a polyoxyethylene-polyoxypropylene block copolymer. In some instances, the non-ionic surfactant is an agent that acts as a stabilizer of a cell membrane. In some instances, the non-ionic surfactant is an agent that protects from cell membrane shearing. In some instances, the non-ionic surfactant is an agent that acts as an anti-foaming agent. In some instances, the non-ionic surfactant comprises pluronic F-68. In some instances, the non-ionic surfactant consists essentially of pluronic F-68. Additional non-limiting examples of non-ionic surfactants contemplated for use in the compositions disclosed herein include alkyl polyglycoside, cetomacrogol 1000, cetostearyl alcohol, cetyl alcohol, cocamide DEA, cocamide MEA, decyl glucoside, decyl polyglucose, glycerol monostearate, IGEPAL CA-630, isoceteth-20, lauryl glucoside, maltosides, monolaurin, mycosubtilin, narrow-range ethoxylate, nonidet P-40, nonoxynol-9, nonoxynols, NP-40, octaethylene glycol monododecyl ether, N-octyl beta-d-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80, and combinations thereof. In some instances, the non-ionic surfactant is present in the composition at a concentration of about 0.01% w/v to about 10% w/v. In some instances, the non-ionic surfactant is present in the composition at a concentration of about 0.1% w/v to about 5% w/v. In some instances, the non-ionic surfactant is present in the composition at a concentration of about 1% w/v to about 5% w/v. In some instances, the non-ionic surfactant is present in the composition at a concentration of about 2% w/v.

Provided herein are methods comprising administering a composition comprising Dengue virus to a subject in need thereof. In some instances, the composition comprises a non-reducing sugar. In some instances, the non-reducing sugar is a sugar capable of trapping water molecules. In some instances, the non-reducing sugar acts as a cryoprotectant, protecting the viability of the Dengue virus during freezing and thawing. In some instances, the non-reducing sugar comprises a disaccharide. In some instances, the non-reducing sugar comprises an alpha, alpha-1, 1-glucoside bond between two alpha glucose units. In some instances, the non-reducing sugar consists essentially of a disaccharide. In some instances, the non-reducing sugar comprises a trehalose. Trehalose is also known as a-D-glucopyranosyl-(1→1)-a-D-glucopyranoside, mycose, and tremalose. In some embodiments, the non-reducing sugar consists essentially of a trehalose. In some instances, the trehalose is alpha-trehalose. In some instances, the trehalose is D-(+)-Trehalose dehydrate. In some instances, the trehalose has the chemical formula of C₁₂H₂₂O₁₁.2H₂O. In some instances, the non-reducing sugar is present in the composition at a concentration of about 5% w/v to about 25% w/v. In some instances, the non-reducing sugar is present in the composition at a concentration of about 1% w/v to about 10% w/v. In some instances, the non-reducing sugar is present in the composition at a concentration of about 10% w/v to about 20% w/v. In some instances, the non-reducing sugar is present in the composition at a concentration of about 15% w/v.

Provided herein are methods comprising administering a composition comprising Dengue virus to a subject in need thereof. In some instances, the composition comprises a carrier protein. Carrier proteins may function as a carrier or stabilizer for steroids, fatty acids, or hormones. In some instances, the carrier protein is a protein capable of stabilizing a virus envelope in storage conditions (e.g., below room temperature). In some instances, the carrier protein is a soluble monomeric protein. In some instances, the carrier protein is albumin. In some instances, the carrier protein is a human protein ensuring compositions disclosed herein are compliant with good manufacturing protocol (GMP) standard. In some instances the carrier protein is human albumin. In some instances, the carrier protein is present in the composition at a concentration of about 0.1% w/v to about 10% w/v. In some instances, the carrier protein is present in the composition at a concentration of about 1% w/v to about 5% w/v. In some instances, the carrier protein is present in the composition at a concentration of about 2% w/v.

Provided herein are methods comprising administering a composition comprising Dengue virus to a subject in need thereof. In some instances, the salt comprises calcium, magnesium, potassium, sodium, boron. In some instances, the salt is a phosphate salt, a chloride salt, a sulfate salt or a dichromate salt. In some instances, the salt is calcium chloride. In some instances, the salt is magnesium chloride. In some instances, compositions comprise calcium chloride and magnesium chloride. In some instances, the salt is present in the composition at a concentration of about 0.1 mM to about 10 mM. In some instances, the salt is present in the composition at a concentration of about 0.1 mM to about 5 mM. In some instances, the salt is present in the composition at a concentration of about 0.1 mM to about 2 mM. In some instances, the salt is present in the composition at a concentration of about 1 mM. In some instances, compositions comprise calcium chloride and magnesium chloride wherein calcium chloride is present in the composition at about 0.1 mM to about 10 mM, and magnesium chloride is present in the composition at about 0.1 mM to about 10 mM. In some instances, compositions comprise calcium chloride and magnesium chloride wherein calcium chloride is present in the composition at about 1 mM, and magnesium chloride is present in the composition at about 1 mM.

Provided herein are methods comprising administering an effective amount of Dengue virus disclosed herein to a subject in need thereof. In some instances, the effective amount is an amount sufficient to increase a level of at least one cytokine in the subject. In some instances, the effective amount is an amount sufficient to increase a level of at least one cytokine in the blood of the subject. In some instances, the effective amount is an amount sufficient to increase a level of at least one cytokine in a serum sample of the subject. In some instances, the effective amount is an amount sufficient to significantly increase the level of the at least one cytokine. In some instances, the effective amount is an amount sufficient to increase the level of the at least one cytokine by about 2% to about 20,000%. In some instances, the effective amount is an amount sufficient to increase the level of the at least one cytokine by about 50% to about 20,000%. In some instances, the effective amount is an amount sufficient to increase the level of the at least one cytokine by about 100% to about 20,000%. In some instances, the effective amount is an amount sufficient to increase the level of the at least one cytokine by about 100% to about 15,000%. In some instances, the effective amount is an amount sufficient to increase the level of the at least one cytokine by about 100% to about 14,000%. In some instances, the effective amount is an amount sufficient to increase the level of the at least one cytokine by about 50% to about 15,000%. In some instances, the effective amount is an amount sufficient to increase the level of the at least one cytokine by about 50% to about 14,000%.

Provided herein are methods comprising administering an effective amount of Dengue virus disclosed herein to a subject in need thereof. In some instances, the effective amount is an amount sufficient to increase a level of at least one cytokine in the subject. In some instances, the at least one cytokine is an interleukin (IL). In some instances, the at least one cytokine is an interferon (IFN). In some instances, the at least one cytokine is an interleukin. In some instances, the at least one cytokine is selected from tumor necrosis factor (TNF) alpha, IFN alpha, IFN beta, IFN gamma, interferon gamma induced protein 10 (IP-10), IL-12, IL-2R, IL-7, IL-15, granulocyte macrophage colony stimulating factor (GM-CSF), and a combination thereof. In some instances the level of TNF alpha is increased from about 50% to about 500%. In some instances the level of TNF alpha is increased from about 50% to about 300%. In some instances the level of TNF alpha is increased from about 50% to about 240%. In some instances the level of IFN alpha is increased from about 50% to about 800%. In some instances the level of IFN alpha is increased from about 50% to about 500%. In some instances the level of IFN alpha is increased from about 50% to about 420%. In some instances the level of IFN beta is increased from about 50% to about 20,000%. In some instances the level of IFN beta is increased from about 50% to about 14,000%. In some instances the level of IFN gamma is increased from about 50% to about 200%. In some instances the level of IFN gamma is increased from about 50% to about 100%. In some instances the level of IP-10 is increased from about 50% to about 8000%. In some instances the level of IP-10 is increased from about 50% to about 5000%. In some instances the level of IP-10 is increased from about 50% to about 4000%. In some instances the level of IL-12 is increased from about 20% to about 200%. In some instances the level of IL-12 is increased from about 20% to about 100%. In some instances the level of IL-12 is increased from about 20% to about 80%. In some instances the level of IL-15 is increased from about 20% to about 200%. In some instances the level of IL-15 is increased from about 20% to about 200%. In some instances the level of IL-15 is increased from about 20% to about 100%. In some instances the level of IL-7 is increased from about 50% to about 1000%. In some instances the level of IL-7 is increased from about 50% to about 1000%. In some instances the level of IL-7 is increased from about 50% to about 500%. In some instances the level of GM-CSF is increased from about 50% to about 1000%. In some instances the level of GM-CSF is increased from about 50% to about 400%. In some instances the level of GM-CSF is increased from about 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, to about 350%. In some instances the level of IL-12R is increased from about 20%, 30%, 40%, 50%, 60%, 70%, 80%, 90%, 100%, 110%, 120%, 130%, 140%, 150%, 160%, 170%, 180%, 190%, to about 200%. In some instances the level of IL-12R is increased from about 20%, 30%, 40%, 50%, 60%, 70%, 80%, 90%, 100%, 110%, 120%, 130%, 140%, 150%, 160%, 170%, 180%, 190%, up to about 200%. Provided herein are methods comprising administering an effective amount of Dengue virus disclosed herein to a subject in need thereof. In some instances, the effective amount is an amount sufficient to increase a level of at least one cytokine in the subject.

Provided herein are methods comprising administering an effective amount of Dengue virus disclosed herein to a subject in need thereof. In some instances, the effective amount is an amount sufficient to increase expression of a protein in tumor cell. In some instances, the effective amount is an amount sufficient to increase expression of a protein expressed on a tumor cell. In some instances, the protein is a checkpoint protein. In some instances, this makes the tumor cell a better target for checkpoint inhibitors. In some instances, the checkpoint protein is programmed death-ligand 1 (PD-L1). In some instances, the effective amount increases the expression of PD-L1 by about 10%, 20%, 30%, 40%, 50%, 60%, 70%, 80%, 90%, up to about 100%. In some instances, the effective amount increases the expression of PD-L1 by about 10% to about 20%. In some instances, the effective amount is an amount sufficient to increase expression of a complex of proteins expressed on a tumor cell. In some instances, the complex is a major histocompatibility complex (MHC). In some instances, the MHC is a Class I MHC. In some instances, the effective amount increases the expression of the MHC by about 10%, 20%, 30%, 40%, 50%, up to about 60%. In some instances, the effective amount increases the expression of the MHC by about 10%, 20%, 30%, 40%, 50%, 60%, 70%, 80%, 90%, up to about 100%. In some instances, the effective amount increases the expression of the MHC by about 10%, 20%, 30%, 40%, 50%, 60%, 70%, 80%, 90%, 100%, 110%, 120%, 130%, 140%, up to about 150%.

Provided herein are methods comprising administering an effective amount of Dengue virus disclosed herein to a subject in need thereof. In some instances, the effective amount is an amount sufficient to increase expression of a protein on a blood cell, such as a lymphocyte, of the subject. In some instances, the effective amount is an amount sufficient to increase expression of a protein on a circulating cell of the subject. In some instances, the blood cell or circulating cell is a T cell. In some instances, the protein is intercellular adhesion molecule (e.g., joins two cells together). In some instances, the intercellular adhesion molecule is intercellular adhesion molecule 1 (ICAM-1). In some instances, ICAM-1 is expressed by endothelial cells and immune system cells such as lymphocytes. ICAM-1 expression on a T cell can be increased by a Dengue virus administration. In some instances, the effective amount increases the expression of ICAM-1 in an immune cell by about 10% to about 500%. In some instances, the expression of ICAM-1 is from about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, or up to about 500%. In some instances, the effective amount increases the expression of ICAM-1 by about 10% to about 300%. In some instances, ICAM-1 is expressed by tumor cells. ICAM-1 expression on a tumor cells can be increased by a Dengue virus administration. In some instances, the effective amount increases the expression of ICAM-1 in a tumor cell by about 10% to about 500%. In some instances, the expression of ICAM-1 is from about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, or up to about 500%. In some instances, the effective amount increases the expression of ICAM-1 by about 10% to about 300%. The level of expression can be measured by an in vitro assay such as flow cytometry.

Provided herein can be a method of treating cancer by administering a Dengue virus to increase an expression of ICAM-1 in an immune cell or in a tumor cell. Increased or persistent ICAM-1 expression may allow for improved cell-cell interaction. A cell-cell interaction can lead to increased binding of an immune cell to a cancer cell.

### Combination Delivery

Provided herein are compositions and methods wherein dendritic cell vaccination is combined with an adjuvant effect of a strain of Dengue virus (DV) to overcome tumor immune evasion mechanisms and deplete tumor cells. Methods described here may be used to treat a subject for cancer by obtaining dendritic cells and tumor cells from the subject, exposing the dendritic cells to the tumor cells or tumor cell lysate, also referred to as “pulsing” the dendritic cells, to primed (or “activated”) the dendritic cells, delivering the resulting primed and tumor-targeting dendritic cells to the subject after the subject has had his/her immune system stimulated with DV (see, e.g., FIGURE. 1). Optionally, the tumor antigen is not from the subject can be used for pulsing the dendritic cells.

Provided herein are methods for treating cancer in a subject in need thereof, comprising: obtaining dendritic cells (DCs); incubating the DCs with at least one tumor cell antigen; administering a Dengue Virus Type 2 serotype strain to the subject; and administering the DCs to the subject. In some instances, the Dengue Virus Type 2 serotype strain is DENV-2 #1710. In some instances, the dendritic cells are autologous dendritic cells. In some instances, the dendritic cells are allogeneic dendritic cells. In some instances, incubating the DCs with at least one tumor antigen comprises incubating the DCs with a tumor cell. In some instances, incubating the DCs with at least one tumor antigen comprises incubating the DCs with a tumor cell lysate.

Dengue virus and dendritic cells disclosed herein to a subject in need thereof. In some instances, methods further comprise administering primed dendritic cells disclosed herein. In some instances, the Dengue virus is initially administered at least 24 hours before administering the dendritic cells. In some instances, the Dengue virus is initially administered between about 12 hours and about 96 hours before administering the dendritic cells. In some instances, the Dengue virus is initially administered between about 24 hours and about 72 hours before administering the primed dendritic cells. In some instances, the Dengue virus is initially administered between 1 day and 4 days before administering the primed dendritic cells. In some instances, the Dengue virus is administered only once. In some instances, the Dengue virus is administered more than once. In some instances, the Dengue virus is administered only before receiving dendritic cells. In some instances, the Dengue virus is administered after receiving the primed dendritic cells. In some instances, the Dengue virus is administered before and after receiving the primed dendritic cells.

In some instances, successful infection or inoculation of the subject with the Dengue virus is confirmed by the development of hyperthermia or fever. In some instances, successful infection or inoculation of the subject with the Dengue virus is confirmed by the presence or increase of circulating cytokines in the blood/plasma of the subject. Cytokines may include, but are not limited to, interleukin-2, interleukin-7, interleukin-12, interleukin-15, interleukin-2R, TNF alpha, IP-10, GM-CSF, interferon-alpha, interferon-beta, and interferon-gamma.

Provided herein are methods comprising administering In some instances, methods described herein comprise administering primed dendritic cells to a subject in need thereof only once. In some instances, the primed dendritic cells are administered more than once. In some instances, the primed dendritic cells are administered a first time and a second time, wherein the first time and the second time are separated by about 1 day, about 2 days, about 3 days, about 4 days, about 5 days, or about 6 days, about 8 days, about 10 days, about 12 days, about 18 days, about 20 days, about 25 days, about 30 days, about 35 days, about 40 days, about 45 days, about 50 days, about 60 days, about 100 days, about 1 year, about 2 years, and any combination thereof. In some instances, the first time and the second time are separated by about 1 week, about 2 weeks, about 3 weeks, or about a month. In some instances, the first time and the second time are separated by more than a month. In some instances, the first time and the second time are separated by less than 12 months. In some instances, the first time and the second time are separated by more than 12 months.

In some instances, primed dendritic cells are administered after the subject has spiked a fever. In some instances, primed dendritic cells are administered after the subject's temperature has risen to between about 37.5° C. and about 42° C. In some instances, the primed dendritic cells are administered after the subject's temperature has risen to between about 38° C. and about 42° C. In some instances, the primed dendritic cells are administered after the subject's temperature has risen to at least about 38.5° C. In some instances, the primed dendritic cells are administered after the subject's temperature has risen to 38.5° C. In some instances, the primed dendritic cells are administered to the subject after the subject's temperature reaches 38 degrees Celsius or higher. In some instances, the subject's temperature is measured by a tympanic or oral method.

### Methods of Administration & Assessment for the Treatment of Melanoma

Provided herein are methods comprising administering a Dengue virus to a subject with melanoma. In some instances, the Dengue virus is strain DV-2 #1710. Further provided herein are methods comprising administering primed dendritic cells disclosed herein to a subject with melanoma. Also provided herein are methods comprising administering Dengue virus and dendritic cells disclosed herein to a subject with melanoma. In some instances, the melanoma is advanced melanoma. In some instances, the subject has unresectable Stage III melanoma. In some instances, the subject has unresectable Stage IV melanoma. In some instances, the subject has a measurable melanoma (e.g., tumor that can be measured in two dimensions). Types and stages of melanoma are further described herein.

In some instances, methods comprise obtaining a tumor sample from the subject with melanoma. In some instances, methods comprise preparing a tumor lysate from the tumor sample. In some instances, methods comprise contacting dendritic cells with the tumor lysate to prime the dendritic cells against melanoma cells of the subject. In some instances, the dendritic cells are allogeneic to the subject. In some instances, the dendritic cells are autologous to the subject. In some instances, methods comprise performing leukapheresis on blood from the subject to obtain the dendritic cell autologous to the subject. In some instances, wherein the methods comprise administering dendritic cells and Dengue virus, leukapheresis may be performed prior to inoculating the subject with the Dengue virus. In some instances, leukapheresis is performed at least one week prior to inoculation with the Dengue virus. In some instances, leukapheresis is performed about one week to about four weeks prior to inoculating the subject with the Dengue virus.

In some methods, methods comprise exercising the subject prior to leukapheresis. In some methods, methods comprise exercising the subject for about 5 hours to about 15 minutes prior to leukapheresis. In some methods, methods comprise exercising the subject for about 1 hour to about 15 minutes prior to leukapheresis. In some instances, methods comprise exercising the subject for 30 minutes prior to leukapheresis. In some instances, exercising comprises an activity that raises the subject's heart rate by at least about 50%. In some instances, exercising comprises an activity that raises the subject's heart rate by at least about 65%. In some instances, exercising comprises an activity that raises the subject's heart rate by at least about 80%. In some instances, exercising comprises an activity that raises the subject's heart rate by at least about 50% to at least about 80%. In some instances, exercising increases the number of dendritic cells obtained.

In some instances, methods comprise administering a dose of Dengue virus, wherein the dose is about 10³ pfu Dengue virus per injection. In some instances, the dose is escalated (e.g., the subject fails to develop Dengue fever). In some instances, methods comprise administering a dose of Dengue virus, wherein the dose is about 10⁴ pfu Dengue virus per injection. In some instances, methods comprise administering a dose of Dengue virus, wherein the dose is about 10⁵ pfu Dengue virus per injection. In some instances, methods comprise administering a dose of Dengue virus, wherein the dose is about 10⁶ pfu Dengue virus per injection. In some instances, methods comprise administering a dose of Dengue virus, wherein the dose is about 10⁷ pfu Dengue virus per injection. In some instances, methods comprise administering about 10³ pfu Dengue virus to 10⁷ pfu Dengue virus per dose.

In some instances, methods comprise administering the dose in a volume of about 500 microliters. In some instances, methods comprise administering the dose in a volume of about 100 microliters to about 1000 microliters. In some instances, methods comprise administering the dose about once a day. In some instances, methods comprise administering the dose about three times per day. In some instances, methods comprise administering the dose about three times per day to about five times per day. In some instances, methods comprise administering the dose three times per day to five times per day.

In some instances, methods comprise administering the Dengue virus via subcutaneous injection. In some instances, methods comprise administering the Dengue virus via intratumoral injection. In some instances, methods comprise administering the Dengue virus via intramuscular injection, intraperitoneal injection, or intravenous injection.

Following injection with the virus, patients are instructed to take oral temperature 3 times per day. Upon onset of fever in excess of 101° F. (38.5° C.) (5-8 days post-injection), patients are admitted for the first DC infusion.

All patients also receive autologous dendritic cells pulsed with autologous tumor lysate.

Approximately 3.0×10⁷ tumor lysate-pulsed DC, (TL-DC), are warmed to 37° C. in a water bath, and infused intravenously over 30 minutes in 0.9% injection-grade NaCl upon admission with febrile symptoms. After 48 hours, the second aliquot of 3.0×10⁷ TL-DC is infused intravenously over 30 minutes concurrent with 0.9% injection-grade NaCl. Optionally, a remaining aliquot of lysate-pulsed DC (6×10⁷) is infused on the 3rd day after presentation of febrile symptoms. The intravenous route provides a simple way for high numbers of DC to traffic to organs such as liver and splenic white pulp, but requires a TH1 cytokine environment for optimum CTL responses. Thus, the first DC infusion occurs on initial presentation of febrile symptoms, in order to utilize the increasing TH1 cytokine levels. The second dose is approximately 48 hours later, to provide a second wave of CTL before the cytokine response shifts to TH2 to prevent a toxic-shock magnitude response. Optionally, an antihistamine is administered to the subject 30 minutes prior to TL-DC infusions to reduce risk of infusion reaction to DMSO. Alternatively, cells are washed on-site to remove DMSO prior to transfer to a Class II infusion bag.

A complete physical examination (including vital signs, weight), evaluation of performance status (i.e. ECOG or Karnofsky) and safety labs are performed at baseline and weekly. Beginning with week 4, immune monitoring and follow-up will occur every 2 weeks until week 12. From week 12-24 patients are evaluated every 3 weeks. After week 24, post-treatment follow-up will occur every 12 weeks until documented disease progression in patients who have stable disease or response.

In some instances, CT or PET scans are performed at 3 to 12 week intervals to determine antitumor activity. CT scans include scans of thoracic, abdominal, and pelvic regions. In some cases, CT or PET scans are performed at 8 week intervals to determine antitumor activity. Alternatively or additionally, biomarkers of disease or anti-tumor activity are characterized. Characterizing biomarkers of disease or anti-tumor activity include: measuring anti-dengue virus neutralizing antibody titers; performing a circulating tumor cells (CTC) assay; performing a circulating melanoma DNA assay; and T cell immunophenotyping/TCR sequencing; detecting anti-nuclear antibodies; and measuring levels of rheumatoid factor. Biopsies, including core biopsies are performed, depending on tumor size and number. Conventional HE histology detects tumor cells undergoing cell death and tumors infiltrated by inflammatory neutrophils or lymphocytes.

Provided herein are methods for preparation of primed dendritic cells (DCs) disclosed herein. Further provided herein are methods for exposing the primed dendritic cells to antigens associated with a disease state, e.g., tumor antigens, resulting primed dendritic cells capable of inducing specific and robust responses from cytotoxic T lymphocyte (CTL) toward cancer cells. Further provided herein are methods for administering such DCs into a subject for treatment of a disorder linked to the disease state. In some instances, the disorder is cancer. In some instances, the disorder is an autoimmune disorder, e.g., rheumatoid arthritis and multiple sclerosis. In some instances, the disorder is a human immunodeficiency virus (HIV) infection or an acquired immunodeficiency syndrome. In some instances, the subject is administered a Dengue Virus prior to administration of the primed DCs.

### Methods of Isolating and Priming Dendritic Cells (DC)

Provided herein are methods that comprise priming dendritic cells, wherein priming the dendritic cells involves contacting the dendritic cells with one or more tumor antigens that are present on target cancer cells. In some cases, the dendritic cells are primed with the tumor antigen alone, the tumor antigen having been synthesized, isolated or purified. Alternatively or additionally, the dendritic cells are primed with a tumor cell lysate, wherein the tumor cell lysate contains the tumor antigen. In some cases, the dendritic cell is primed with a whole cancer cell expressing the tumor antigen. The dendritic cell is then administered to the subject, where it will present the tumor antigen to the CTL, and thus, tailor the CTL for recognition and destruction of target cancer cells.

Provided herein are methods which limit dendritic cells exposure to polymers present in a plastic container material. For example, in the case of soft plastic bags, polymers may leach into the media solution and impact DC activity. Instead, dendritic cells may be cultured, stored and shipped in and on a hard container, such as a polystyrene tissue culture plate. This avoids a reduction in dendritic cell immunostimulatory activity that can be caused by exposure to polymers contained in soft plastic bags. For example, these polymers can reduce the amount of IL-12 produced by the dendritic cells, thereby reducing their capacity to induce a robust CTL response. Examples provided herein demonstrate that primed dendritic cells generated by the methods disclosed herein are capable of secreting at least 18 pg/mL of IL-12p70, whereas dendritic cells produced by standard methods typically only produce 4-6 pg/mL of IL-12p70.

In some instances, it is desirable or advantageous to prime the dendritic cells with a tumor lysate. Notably, the methods disclosed herein utilize a gentle cell lysis protocol that preserves the integrity of the tumor antigen. This gentle lysis may be achieved by exposing the tumor or cancer cells to a calcium or sodium hypochlorite solution for no more than about 30-60 minutes. Similarly, any tumor cells used to prime dendritic cells are disassociated gently, for instance, by a Miltenyi GentleMACS system, or the like.

Provided herein are primed dendritic cells prepared by the methods disclosed herein, wherein the methods comprise administering the primed dendritic cells to the subject along with an agent that boosts the subject's immune system. The combination of primed dendritic cells with a viral infection provides for an effective treatment with minimal administration, possibly as few as one time, which avoids the challenge of subject adherence to therapy. The primed dendritic cells may be autologous, meaning derived from a subject's own cells, or allogenic, derived from another subject with a similar tissue type.

Provided herein are methods that comprise priming DCs and administering the primed DCs to a subject in need thereof, wherein the DCs induce a response from cytotoxic T lymphocytes (CTL) resulting in cytotoxicity of target cells. The DCs may comprise allogeneic dendritic cells or autologous dendritic cells. In some instances, the methods described herein comprise administering allogeneic primed dendritic cells to a subject. In some instances, the methods described herein comprise administering autologous primed dendritic cells to a subject. The methods disclosed herein comprising administering primed DCs to the subject may be referred to herein as “dendritic cell vaccination.”

In some instances, methods described herein comprise obtaining dendritic cells from CD34+ progenitor cells in the bone marrow. In some instances, methods described herein comprise obtaining dendritic cells from CD1+CD14+ immature monocytes in the peripheral blood. In some instances, obtaining the dendritic cells comprises leukapheresis. In some instances, leukapheresis comprises withdrawing a unit of blood from the subject or a donor, separating a series of blood-components: red cells, platelets, and most of the plasma factors, which are returned to the subject, with the white blood cells remaining. In some instances, methods described herein comprise testing the white blood cells for sterility, shipping or storing them cold (4° C.), and or processing the DCs from the apheresis product.

Provided herein are methods of producing DCs, wherein the methods comprise separating monocytes in the unit of blood from other white cells, including, but not limited to, T cells, B cells, NK cells, Eosinophils and Basophils. This may be accomplished with immuno-magnetic selection or by adherence properties. Immuno-magnetic selection involves contacting white blood cells from the unit of blood with a sterile plastic column with plastic beads coated with antibodies for immune cells, such as, by way of non-limiting example, CD surface proteins: (CD4, CD8, CD56, etc.). Unwanted (non-monocyte) cells will adhere to the beads, leaving the monocytes to pass through and be collected. In positive selection, magnetic beads may be coated with antibodies for CD1 and/or CD14 to capture monocytes, a magnet is placed against the column, and unwanted cells are flushed out of the column with a buffered saline solution or cell-viable media. The monocytes are then washed off the beads and collected in a following step. In adherence selection, the properties of monocytes to stick to certain surfaces are used to separate them by running the apheresis product down a slanted column.

Provided herein are methods for cell collection which may comprise collecting only a few thousand monocytes from the unit of blood. Currently employed methods of immunotherapy generally requires DC doses in the range of 50 million. Thus, methods disclosed herein may comprise expanding monocytes, as well as any precursors thereof, and any cells differentiated therefrom (e.g., DCs). Expanding cells may comprise contacting cells with factors such as growth factors, colony-stimulation factors, cytokines, or any other proliferation or growth inducing factors, and combinations thereof. By way of non-limiting example, the recombinant human growth factors rhulnterleukin-4 (IL-4), and rhuGranulocyte-Macrophage-Colony-Stimulation Factor (GM-CSF), may be used to accomplish the expansion of DC numbers. In addition, IL-4 and GM-CSF may be required to develop mature DCs from monocytes, which have poor antigen-uptake and CTL-stimulating ability, compared to mature DCs. Thus, IL-4 and GM-CSF may expand the number and the development of mature-DC markers. DC markers may include, but are not limited to CD11, CD80, and CD83, as well as increased expression of both Class I (for presentation of short peptides to CD8+ cells), and Class II (for presentation of longer peptides to CD4+Helper-Inducer T lymphocytes) MHC complexes. Expanding cells may produce mature DCs in the tens of millions within about 2 days, Expanding cells may produce mature DCs in the tens of millions within about 3 days, Expanding cells may produce mature DCs in the tens of millions within about 4 days, Expanding cells may produce mature DCs in the tens of millions within about 5 days, or Expanding cells may produce mature DCs in the tens of millions within about one week.

In some instances, methods described herein comprise contacting or pulsing DCs with peptides/antigens, tumor cells, tumor supporting cells, tumor cell lysate and/or tumor supporting cell lysate. The term “pulsing,” as used herein, generally refers to contacting DCs more than once at one or more intervals, and may be used interchangeably with contacting, unless specified otherwise. In some instances, the methods comprise contacting or pulsing DCs with a peptide that binds MHC Class I molecules (“MHC Class I peptide”). In some instances, methods described herein comprise contacting or pulsing DCs with a peptide that binds MHC Class II molecules (“MHC Class II peptides”). In some instances, methods described herein comprise contacting or pulsing DCs with MHC Class I peptides and MHC Class II peptides. In some instances, the contacting or pulsing makes the DCs competent to prime CTL and target CTL to tumors. In some instances, methods described here comprise contacting or pulsing DCs with manufactured/synthetic Class I and/or Class II peptides. In some instances, the Class I and/or class II peptides are manufactured, then added to the DC medium, optionally in in microgram quantities or less. In some instances, methods described herein include Class II peptides for a sustained immune response. In some instances, methods described herein comprise DNA or RNA sequencing of the peptide (i.e. tumor antigen) and/or using electroporation to insert the DNA or RNA into the DCs to trigger antigen processing. In some instances, methods described herein do not require HLA matching of DCs. In some instances, the peptide or portion thereof is represented by an amino acid sequence selected from EGSRNQDWL (SEQ ID NO: 1), (TAYRYHLL) (SEQ ID NO: 2), or combinations thereof.

In some instances, the peptides disclosed herein are Class I peptides. Class I peptides may by manufactured, then added to the DC medium in microgram quantities. However, this technique is costly, because the peptides must be matched to the subject's HLA type, and if the tumor cell does not present that antigen, it can evade detection and lysis. The lack of Class II peptides to activate CD4+ help leads to rapid decline of immune response power. Other methods may comprise RNA sequencing of common tumor antigens, then using electroporation to insert the RNA into the DCs to trigger antigen processing. This method does not require HLA matching, and includes Class II peptides for a sustained immune response. However, RNA sequencing may be technically complex, and may only present a limited number of antigens of thousands of potential gene products. For these reasons, autologous whole-tumor cells or their lysate have the advantages of low cost, ready availability by biopsy (1-2 gm sufficient), and contain the full array of potential antigens for a broad and deep immune response.

Provided herein are methods for priming dendritic cells, comprising obtaining whole tumor cells and/or lysates thereof. Tumor cells may be killed by radiation or other means and preparing lysate by various methods. In some instances, lysing the tumor cells does not comprise trypsin enzyme digestion and freeze-thaw cycles, which are simple and fast, but can damage the delicate peptides within. The methods disclosed herein may employ an automated cell processor (e.g., the Miltenyi GentleMACS system), which allows the sample to be manually minced, suspended in PBS solution, then a pre-selected tissue-specific software-controlled rotor system separates the tumor cells. The single-cell suspension may be membrane-lysed with minimal damage to tumor peptides.

In some instances, methods described herein comprise contacting the dendritic cells with autologous tumor cells or lysates thereof. In some instances, methods described herein comprise contacting the dendritic cells with autologous whole-tumor cells (e.g., tumor cells and tumor supporting cells) or lysates thereof which contain the full array of potential antigens for a broad and deep immune response. Methods for dendritic cell priming described herein may comprise contacting the dendritic cells with tumor cell lysate comprising apoptotic or necrotic bodies. In further instances, the tumor cell lysate comprises tumor antigens from the microenvironment surrounding the tumor cells, such as extracellular matrix proteins.

In some instances, methods described herein comprise contacting the DCs with an augmenting agent that will augment the priming, proliferation or viability of the DCs. By way of non-limiting example, the augmenting agent may be selected from lymphokines, monokines, cytokines, growth factors, cells, cell fragments, (non-protein) small molecules, antibodies, antibody fragments, nucleic acids, and combinations thereof.

In some instances, methods described herein for preparing cells and antigens for DC priming comprises rendering the target cells (e.g., cancer cells) incapable of cell division. For example, the methods may comprise treating cells with mytomycin C or radiation to render cells incapable of cell division. These may include cells that are added as augmenting agents or cells used to pulse DCs (e.g., tumor cells).

In some instances, methods described herein comprise pulsing the DCs from about 1 hour to about 24 hours. In some instances, methods described herein comprise pulsing the DCs from about 12 hours to about 48 hours. In some instances, methods described herein comprise pulsing the DCs from about 8 hours to about 24 hours. In some instances, methods described herein comprise pulsing the DCs for about 18 hours. Pulsing may comprise contacting the DCs at least once with the peptides/antigens, tumor cells, tumor supporting cells, tumor cell lysate and/or tumor supporting cell lysate. Pulsing may comprise contacting the DCs at least twice with the peptides/antigens, tumor cells, tumor supporting cells, tumor cell lysate and/or tumor supporting cell lysate. Pulsing may comprise contacting the DCs at least three times with the peptides/antigens, tumor cells, tumor supporting cells, tumor cell lysate and/or tumor supporting cell lysate. Pulsing may comprise contacting the DCs less than two times, less than three times, less than four times, less than five times, or less than 10 times with the peptides/antigens, tumor cells, tumor supporting cells, tumor cell lysate and/or tumor supporting cell lysate. Pulsing may comprise adding the peptides/antigens, tumor cells, tumor supporting cells, tumor cell lysate and/or tumor supporting cell lysate to the DCs more than once, such that the peptides/antigens, tumor cells, tumor supporting cells, tumor cell lysate and/or tumor supporting cell lysate accumulates in the DC culture media. Pulsing may comprise washing the cells or removing the DC culture media between one or more pulses.

In some instances, methods described herein comprise contacting DCs with a maturing agent described herein to enhance, complete or finalize the maturation of the DCs. In some embodiments, the maturing agent also acts as a “danger signal.” Without this danger signal, the tumor antigen may induce Treg production or activity, which will ultimately lower CTL activity. In some embodiments, the maturing agent/danger signal is an inflammatory signal. The inflammatory signal may also be referred to as an inflammatory mediator. Inflammatory mediators may include cytokines, as well as other factors (e.g., chemokines, adhesion molecules, etc.), that may not be classified by those in the art as cytokines, but affect inflammation either directly or indirectly, In some embodiments, the inflammatory mediator is selected from a chemokine, a cytokine, a pathogen, a non-peptidic small molecule, a compound, an antibody, a peptide, fragments thereof, portions thereof, and combinations thereof. In some embodiments, the inflammatory signal is a modulator of a pattern recognition receptor (PRR) or pathway thereof.

In some instances, inflammatory signals described herein are selected from an interferon, a toll-like receptor signaling modulator, and combinations thereof. By way of non-limiting example, the interferon may be interferon-gamma. In some embodiments, the inflammatory signal is a toll-like receptor signaling pathway modulator.

In some instances, inflammatory signals described herein are toll-like receptor (TLR) signaling pathway regulators. By way of non-limiting example, the toll-like receptor signaling pathway regulator may be lipopolysaccharide (LPS), a polysaccharide from bacterial cell walls. In some instances, the toll-like receptor signaling pathway regulator may be selected from a toll-like receptor signaling pathway regulator that regulates TLR1, TLR2, TLR3, TLR4, TLR5, TLR6, TLR7, TLR8, TLR9 and TLR 10. The toll-like receptor signaling pathway regulator may be a ligand, a binding protein, an antibody, an agonist or an antagonist, of a TLR. The toll-like receptor signaling pathway regulator may be selected from a peptide, a protein, a cell fragment, a cell-wall component, a lipoprotein, a peptidoglycan, a polysaccharide, a monosaccharide, and a small molecule compound. The toll-like receptor signaling pathway regulator may be a portion of an animal cell, a plant cell, a bacterial cell, a yeast cell, a fungal cell, and combinations thereof. The toll-like receptor signaling pathway regulator may be a TLR2 signaling pathway regulator. By way of non-limiting example, the TLR2 signaling pathway regulator may be lipoteichoic acid, MALP-2, MALP-4, OspA, Porin, LcrV, lipomannan, GPI anchor, lysophosphatidylserine, lipophosphoglycan, glycophosphatidylinositol, zymosan, hsp60, and hemagllutinin.hemagglutinin. The toll-like receptor signaling pathway regulator may be a TLR4 signaling pathway regulator. By way of non-limiting example, the TLR4 signaling pathway regulator may be buprenorphine, carbamazepine, ethanol, fentanyl, levorphanol, LPS, methadone, morphine, oxcarbazepine, oxycodone, pethidine, and glucuronoxylomannan. The toll-like receptor signaling pathway regulator may be a TLR7 signaling pathway regulator. By way of non-limiting example, the TLR7 signaling pathway regulator may be a single stranded RNA or an imidazoquinoline compound. The toll-like receptor signaling pathway regulator may be a TLR8 signaling pathway regulator. By way of non-limiting example, the TLR8 signaling pathway regulator may be a single stranded RNA, a G-rich oligonucleotide or an imidazoquinoline compound. The imidazolquinoline compound may be R848. After exposure to the inflammatory signal, the DCs may up-regulate their CD80/CD83+activation markers, increase production of IL-12p70 to induce a Type 1 CTL response, and become resistant to further antigen uptake and processing.

In some instances, methods described herein comprise contacting DCs with a maturing agent described herein to enhance, complete or finalize the maturation of the DCs. In some instances, the agent to finalize the maturation of the DCs comprises LPS bacterial cell wall. In some instances, the maturation agents comprise IFN-gamma. In some instances, the maturation agents comprise R848. In some instances, the maturation agents comprise CD40L. In some instances, the maturation agents comprise a combination of at least any two agents selected from LPS bacterial cell wall, IFN-gamma, R848 and CD40L. In some instances, the maturation agents comprise a combination of at least any three agents selected from LPS bacterial cell wall, IFN-gamma, R848 and CD40L. In some instances, the maturation agents comprise LPS bacterial cell wall, IFN-gamma, R848, CD40L, or any combination thereof. In some instances, the maturation agents are administered simultaneously. In some instances, the maturation agents are administered sequentially. In some instances, the maturation agents are administered sequentially starting with LPS being administered first. In some instances, the maturation agents are administered sequentially starting with IFN-gamma being administered first. In some instances, the maturation agents are administered sequentially starting with R848 being administered first. In some instances, the maturation agents are administered sequentially starting with LPS and IFN-gamma being administered simultaneously first. In some instances, the maturation agents are administered sequentially with LPS and IFN-gamma being administered simultaneously first followed by administration of R848, CD40L, or any combination thereof. In some instances, the maturation agents are administered sequentially with LPS and IFN-gamma being administered simultaneously first followed by administration of R848. In some instances, the maturation agents are administered sequentially with LPS bacterial cell wall and IFN-gamma being administered simultaneously first followed by administration of R848, and then of CD40L.

Provided herein are methods for producing primed dendritic cells described herein, wherein the methods comprise contacting primed dendritic cells with interferon gamma. In some embodiments, the methods comprise culturing the primed dendritic cells in a culture media with a concentration of interferon gamma selected from about 100 U/mL to about 10,000 U/mL, about 500 U/mL to about 5000 U/mL, and about 500 U/mL to about 2,000 U/mL. In some embodiments, the methods comprise culturing the primed dendritic cells in a culture media with a concentration of interferon gamma of about 500 U/mL. In some embodiments, the methods comprise culturing the primed dendritic cells in a culture media with a concentration of interferon gamma of about 1000 U/mL. In some embodiments, the methods comprise culturing the primed dendritic cells in a culture media with a concentration of interferon gamma of about 2000 U/mL.

In some instances, methods for producing primed dendritic cells described herein may comprise contacting primed dendritic cells with TLR8 agonist R848. In some embodiments, the methods comprise culturing the primed dendritic cells in a culture media with a concentration of R848 selected from about 0.1 μg/mL to about 50 μg/mL, about 1 μg/mL to about 20 μg/mL, and about 1 μg/mL to about 10 μg/mL. In some embodiments, the methods comprise culturing the primed dendritic cells in a culture media with a concentration of R848 of about 1 μg/mL. In some embodiments, the methods comprise culturing the primed dendritic cells in a culture media with a concentration of R848 of about 5 μg/mL. In some embodiments, the methods comprise culturing the primed dendritic cells in a culture media with a concentration of R848 of about 10 μg/mL.

In some instances, methods for producing primed dendritic cells described herein comprise contacting primed dendritic cells with lipopolysaccharide. In some embodiments, the methods comprise culturing the primed dendritic cells in a culture media with a concentration of lipopolysaccharide selected from about 1 ng/mL to about 100 ng/mL, about 1 ng/mL to about 50 ng/mL, and about 1 ng/mL to about 25 ng/mL. In some embodiments, the methods comprise culturing the primed dendritic cells in a culture media with a concentration of lipopolysaccharide of about 5 ng/mL. In some embodiments, the methods comprise culturing the primed dendritic cells in a culture media with a concentration of lipopolysaccharide of about 10 ng/mL. In some embodiments, the methods comprise culturing the primed dendritic cells in a culture media with a concentration of lipopolysaccharide of about 15 ng/mL.

Provided herein are methods that comprise sterility, specificity, and viability testing of primed DCs produced by the methods disclosed herein. The testing may occur before shipping or storing the DC. The testing may occur after shipping or storing the DC. The methods may comprise measuring expression level of IL-12p70 in DC, either at the RNA or protein level. IL-12p70 is an independent predictor of clinical response, tested across numerous trials in the last two decades, some with approximately 40% response rates. The expression level of IL-12p70 in primed DCs produced by the methods disclosed herein may be at least about two times greater than primed DCs produced/stored/shipped by traditional methods. The expression level of IL-12p70 in primed DCs produced by the methods disclosed herein may be at least about two times greater than primed DCs produced/stored/shipped by traditional methods (“traditional primed DCs”). The expression level of IL-12p70 in primed DCs may be at least about 10%, at least about 20%, at least about 30%, at least about 40%, at least about 50%, at least about 60%, at least about 70%, at least about 80%, at least about 90%, or at least about 100% greater than traditional primed DCs. The expression level of IL-12p70 in primed DCs may be at least about three times greater than traditional primed DCs. The expression level of IL-12p70 in primed DCs may be at least about four times greater than traditional primed DCs. The expression level of IL-12p70 in primed DCs produced by the methods disclosed herein may be about two to about twenty times greater than traditional primed DCs.

Provided herein are methods for producing dendritic cells that produce more than 166 ng/mL of IL-12p70. Also provided herein are dendritic cells that produce more than 2010 ng/mL of IL-12p70. The DCs of the present application may produce at least about 1510 ng/mL, at least about 12 ng/mL, at least about 1914 ng/mL, at least about 16 ng/mL, at least about 18 ng/mL, at least about 20 ng/mL, at least about 22 ng/mL, at least about 24 ng/mL, at least about 26 ng/mL, at least about 28 ng/mL, at least about 29 ng/mL, or at least about 30 ng/mL. The DCs of the present application may produce from about 20 ng 10 ng/mL to about 30 ng/mL. The DCs of the present application may produce from about 20 ng 10 ng/mL to about 29 ng/mL. The DCs of the present application may produce from about 15 ng/mL to at least about 29 ng/mL.

### CTL Response

Provided herein are methods for producing DCs described herein, comprising testing the ability of the DCs to induce a CTL response. Measuring the level of the CTL response may comprise measuring cytokines or inflammatory mediators in blood, serum or plasma from the subject. Measuring the level of the CTL response may comprise measuring a change in the level of a cytokine or inflammatory mediator in blood, serum or plasma from the subject. Measuring the level of the CTL response may comprise measuring the production of a cytokine or inflammatory mediator in vitro. Cytokines and inflammatory mediators may include interleukins, migration inhibitory proteins, monocyte chemotactic proteins, monocyte chemoattractant proteins, interferons, tumor necrosis factors, colony stimulating factors (CSFs), macrophage inflammatory proteins, monokines, chemokines, chemokine ligands (CCLs), and C—X—C motif chemokines (CXCL), and receptors thereof. Cytokines and inflammatory mediators include, but are certainly not limited to, interleukin 1 beta (IL-1b), interleukin 2 (IL-2), interleukin 4 (IL-4), interleukin 5 (IL-5), interleukin 7 (IL-7), interleukin 8 (IL-5), interleukin 10 (IL-10), interleukin 13 (IL-13), interleukin 6 (IL-6), interleukin 12 (IL-12), interleukin 15 (IL-15), interleukin 17 (IL-17), Rantes, Eotaxin, macrophage inflammatory protein 1 alpha (MIP-1a), macrophage inflammatory protein 1 beta (MIP-1b), granulocyte macrophage colony-stimulating factor (GM-CSF), monocyte chemoattractant protein-1 (MCP-1), interferon alpha (IFNα), interferon gamma (IFNg), interleukin 1 receptor alpha (IL-1Ra), interleukin 2 receptor (IL-2R), tumor necrosis factor alpha (TNFα), interferon gamma induced protein (IP-10), and monokine induced by gamma interferon (MIG). CTL response may be measured by expression of tumor response genes (MxA, etc.), enabling high cancer killing (turning “cold” tumors “hot”), and generating further tumor shrinkage in non-responder or low responders.

### Hard Surface

Provided herein are methods for preparing DCs described herein, comprising culturing the DCs on a hard surface. The term, “hard surface,” as used herein, generally refers to a standard plastic tissue culture plate or flask (e.g., a polystyrene plate). The methods disclosed herein comprise culturing DCs on a hard surface to which the DCs can adhere. In some embodiments, the hard surface is coated with a protein, peptide, extracellular matrix molecule, polymer, or combinations thereof. In some embodiments, the hard surface is not coated (e.g., the DCs adhere directly to the hard plastic surface). The hard surface is contrasted to a soft tissue culture bag, also known as cell differentiation bags. Soft tissue culture bags may be bags comprising polymers or chemicals (e.g., phthalates) that reduce the DC's Type 1 response capability. Soft tissue culture bags may be bags comprising polymers or chemicals that evoke a neutral Type 0 response from the DCs, rendering the DCs functionally inert. Soft tissue culture bags may be bags comprising a polymer selected from polyethylene, fluorinated ethylene propylene (FEP), hexafluoropropylene, tetrafluoroethylene, polytetrafluoroethylene, and co-polymers thereof, and combinations thereof.

Provided herein are methods for preparing DCs described herein, comprising transferring the DCs to a storage unit. The storage unit may also be a shipping unit. The storage unit may be selected from a flexible or soft container or surface (e.g., a bag) or a hard container or surface (e.g., a flask or plate). The storage unit may comprise a hard plastic surface. The storage unit may consist essentially of a hard plastic surface. The storage unit may consist of a hard plastic surface. The storage unit may comprise a non-plastic surface (e.g., glass). The storage unit may consist essentially of a non-plastic surface. The storage unit may consist of a non-plastic surface. The storage unit may be free of any polymers that would be taken up by, and/or induce a response in, cells stored within the storage unit. The storage unit may be free or essentially free of polymers that induce a neutral or Type 0 response in immature DCs. A neutral response may be characterized by low expression of IL-12p70. The storage unit may be essentially free of any polymers that would be taken up by, and/or induce a response in, cells stored within the storage unit. Essentially free may mean that the storage unit is at least 90%, at least 95%, at least 98%, or at least 99% free of any polymers that would be taken up by, and/or induce a response in, cells stored within the storage unit. Essentially free may mean that the storage unit is at least 99.5%, at least 99.6%, at least 99.7%, at least 99.8%, or at least 99.9% free of any polymers that would be taken up by, and/or induce a response in, cells stored within the storage unit.

In some instances, the storage units comprise an inner surface, wherein the inner surface is the surface of the storage unit that is in contact with cells stored therein. The inner surface may consist of a hard plastic surface. The inner surface may be glass. The inner surface may be absent of any polymers that would be taken up by, and/or induce a response in, cells stored within the storage unit. The inner surface may be constructed of polymers that are not taken up by immature DCs or any cells stored within the storage unit. The inner surface may be free of any polymers that would be taken up by, and/or induce a response in, cells stored within the storage unit. The inner surface may be essentially free of any polymers that would be taken up by, and/or induce a response in, cells stored within the storage unit. The inner surface may be at least 90%, at least 95%, at least 98%, or at least 99% free of any polymers that would be taken up by, and/or induce a response in, cells stored within the storage unit following addition of cells and storage media. The inner surface may be at least 99.5%, at least 99.6%, at least 99.7%, at least 99.8%, or at least 99.9% free of any polymers that would be taken up by, and/or induce a response in, cells stored within the storage unit following addition of cells and storage media. The inner surface may be free or essentially free of polymers that induce a neutral or Type 0 response in immature DCs. A neutral response may be characterized by low expression of IL-12p70.

Provided herein are methods for storing DCs produced by the methods described herein, wherein the storage units are suitable for freezing at −70° C. in liquid N2, storage up to 1 year, and shipping to the clinic for use. The methods may comprise storing and/or shipping mature DCs, immature DCs, monocytes or blood in a storage unit. The methods may comprise shipping cells cool overnight. The methods may comprise thawing or warming cells to 37° C. (e.g., in a warm-water bath).

### Methods of Isolating and Lysing Tumor Cells

Provided herein are methods for treating a subject, comprising administering the DCs produced by the methods disclosed herein to target tumor cells. In some instances, DCs are primed with tumor cells from a subject. In some instances, the tumor cells are isolated cells from a tumor microenvironment of the subject, referred to herein as tumor supporting cells. In some instances, dendritic cells are exposed to/pulsed with tumor cells, tumor supporting cells and/or peptides thereof, such that the dendritic cells will target tumor cells and/or tumor supporting cells that support tumor growth and metastasis (e.g., endothelial cells, vascular cells, immune cells, etc.). In some instances, peptides/antigens from tumor cells and tumor supporting cells induce dendritic cells or cytotoxic lymphocytes with receptors for peptides/antigens on both tumor cells and tumor supporting cells, resulting in targeting of the dendritic cells or cytotoxic lymphocytes to the tumor microenvironment rather than only the tumor cells. In some instances, tumor cells and/or tumor supporting cells are obtained from a biopsy of tumor tissue. In some instances, the biopsy comprises cells selected from tumor cells, adipocytes, fibroblasts, endothelial cells, infiltrating immune cells, and combinations thereof. In some embodiments, the methods comprise expanding tumor cells in order to have a sufficient number of tumor cells, tumor cell lysates or tumor cell antigens to effectively and optimally prime/pulse the DCs. Expanding may comprise proliferating of the tumor cells in vitro.

Provided herein are methods for activating DCs disclosed herein to target tumor cells, wherein the DCs are activated with lysed tumor cells and/or tumor supporting cells and surrounding extracellular matrix. In some instances, lysing comprises contacting the tumor cells and/or tumor supporting cells with an NH4Cl enzyme solution to eliminate red blood cells. In some instances, the lysing comprises contacting the tumor cells and/or tumor supporting cells with hypochlorous acid solution to induce immunogenic cell death. In some instances, the cells are lysed gently enough to not destroy peptides. In some instances, the cells are lysed to produce apoptotic or necrotic bodies. In some instances, the methods comprise lysing the tumor cells and/or tumor supporting cells with an enzymatic solution. In some instances, the methods comprise lysing the tumor cells and/or tumor supporting cells with a peroxide-free solution or a low peroxide-containing solution.

Provided herein are methods for activating DCs disclosed herein comprising lysing the tumor cells with a hypochlorite solution (HOCL). In some instances, the hypochlorite solution comprises sodium chlorite. In some instances, the hypochlorite solution comprises calcium chlorite. In some instances, the concentration of the hypochlorite in a media in which the tumor cells are suspended is about 10 μM, about 20 μM, about 30 μM, about 40 μM, about 50 μM, about 60 μM, about 70 μM, about 80 μM, about 90 μM, or about 100 μM.

Provided herein are methods for methods activating DCs produced by the methods described herein, wherein the methods comprise lysing the tumor cells and/or tumor supporting cells with a detergent solution prior to contact with the DCs. In some instances, the detergent is selected from, but is not limited to, Triton X-100, Triton X-114, NP-40, Brij-35, Brij-58, Tween 20, Tween 80, octyl glucoside, octyl thioglucoside, SDS, CHAPS, and CHAPSO. In some instances, the detergent solution is purified of peroxides, and other impurities. In some instances, the detergent is about 0.1% to about 10% v/v of the detergent solution. In some instances, the detergent is about 0.1% to about 5% v/v of the detergent solution. In some instances, the detergent is about 0.5% to about 5% v/v of the detergent solution. In some instances, the detergent is about 1% to about 10% v/v of the detergent solution. In some instances, the detergent is about 1% to about 5% v/v of the detergent solution. In some instances, the methods comprise lysing cells without shaking, vortexing, freezing, thawing, shear pressure, sonicating and/or heating the cells.

In some instances, the methods for cell lysis described herein further comprise stopping or neutralizing the lysing. For example, cells may be washed with a buffered saline solution (phospho-buffered saline solution or Hank's balanced salt solution) to neutralize the lysing.

### Kits

Disclosed herein can be kits comprising compositions. Disclosed herein can also be kits for the treatment or prevention of a cancer, pathogen infection, or immune disorder. In some cases, a kit can include a therapeutic or prophylactic composition containing an effective amount of a composition of Dengue virus in unit dosage form. In some cases, a kit comprises a sterile container which can contain a therapeutic composition of Dengue virus; such containers can be boxes, ampules, bottles, vials, tubes, flasks, bags, pouches, blister-packs, or other suitable container forms known in the art. Such containers can be made of plastic, glass, laminated paper, metal foil, or other materials suitable for holding medicaments. In some cases, a kit can include cells, such as dendritic cells, from about 1×104 cells to about 1×1012 cells. In some cases a kit can include at least about 1×105 cells, at least about 1×106 cells, at least about 1×107 cells, at least about 4×107 cells, at least about 5×107 cells, at least about 6×107 cells, at least about 6×107 cells, at least about 8×107 cells, at least about 9×107 cells, at least about 1×108 cells, at least about 2×108 cells, at least about 3×108 cells, at least about 4×108 cells, at least about 5×108 cells, at least about 6×108 cells, at least about 6×108 cells, at least about 8×108 cells, at least about 9×108 cells, at least about 1×109 cells, at least about 2×109 cells, at least about 3×109 cells, at least about 4×109 cells, at least about 5×109 cells, at least about 6×109 cells, at least about 6×109 cells, at least about 8×109 cells, at least about 9×109 cells, at least about 1×1010 cells, at least about 2×1010 cells, at least about 3×1010 cells, at least about 4×1010 cells, at least about 5×1010 cells, at least about 6×1010 cells, at least about 6×1010 cells, at least about 8×1010 cells, at least about 9×1010 cells, at least about 1×1011 cells, at least about 2×1011 cells, at least about 3×1011 cells, at least about 4×1011 cells, at least about 5×1011 cells, at least about 6×1011 cells, at least about 6×1011 cells, at least about 8×1011 cells, at least about 9×1011 cells, or at least about 1×1012 cells. For example, about 5×1010 cells can be included in a kit. In another example, a kit can include 3×106 cells; the cells can be expanded to about 5×1010 cells and administered to a subject. Such kits can further comprise instructions for use thereof.

### Pharmaceutical Compositions

Provided herein are pharmaceutical compositions comprising an effective amount of a Dengue virus disclosed herein. In some instances, the pharmaceutical compositions comprise more than one strain of Dengue virus. In some instances, the pharmaceutical compositions comprise at least a portion of a Dengue virus. The portion of the Dengue virus may be a portion sufficient to generate an immune response in a subject receiving the pharmaceutical composition. The compositions may further comprise one or more pharmaceutically acceptable salts, excipients or vehicles. Pharmaceutically acceptable salts, excipients, or vehicles for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

In some instances, the carriers disclosed herein comprise neutral buffered saline. The pharmaceutical compositions may include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also may be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers may be conventional buffers such as acetate, borate, citrate, phosphate, bicarbonate, or Tris-HCl. Acetate buffer may be about pH 4-5.5, and Tris buffer may be about pH 7-8.5.

Provided herein are compositions that comprise a Dengue virus, wherein the composition is in liquid form, lyophilized form or freeze-dried form and may include one or more lyoprotectants, excipients, surfactants, high molecular weight structural additives and/or bulking agents. In some instances, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the virus upon lyophilization. Exemplary lyoprotectant concentrations for sugars (e.g., sucrose, lactose, trehalose) in the pre-lyophilized formulation are from about 10 mM to about 400 mM.

Provided herein are compositions that comprise a Dengue virus disclosed herein, wherein the compositions are suitable for injection or infusion. Exemplary compositions are suitable for injection or infusion into an animal by any route available to the skilled worker, such as intraarticular, subcutaneous, intratumoral, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

Provided herein are pharmaceutical compositions that comprise a Dengue virus disclosed herein, and a non-aqueous solvent. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, anti-oxidants, chelating agents, inert gases and the like.

Provided herein are pharmaceutical compositions that comprise a Dengue virus disclosed herein, wherein the pharmaceutical composition is formulated for inhalation, such as for example, as a dry powder. Suitable and/or preferred pharmaceutical formulations may be determined in view of the present disclosure and general knowledge of formulation technology, depending upon the intended route of administration, delivery format, and desired dosage. Regardless of the manner of administration, an effective dose may be calculated according to patient body weight, body surface area, or organ size. Further refinement of the calculations for determining the appropriate dosage for treatment involving each of the formulations described herein are routinely made in the art and is within the ambit of tasks routinely performed in the art. Appropriate dosages may be ascertained through use of appropriate dose-response data.

### EXAMPLES

### Example 1. Generation and Pulsing of Murine Dendritic Cells (DCs)

A method as described by Lutz M., et. al. (J. Immunol. Methods 223:77-92, 1999), was employed to generate mature DCs from mouse bone marrow. Bone marrow suspensions were incubated in petri dishes in medium supplemented with recombinant murine GM-CSF for 10 days. Non-adherent cells were collected, centrifuged and resuspended in medium containing GM-CSF and lipopolysaccharide. Two days later, the DCs were harvested and their viability was determined by trypan-blue exclusion. Purity of the DCs was determined by flow cytometry analysis. DCs were pulsed with the synthetic peptides at 10 μg/ml for 18 hours. After 18 hours of incubation, DCs were harvested, washed twice in HBSS, and resuspended in HESS for additional analysis (see Example 2 and 3).

### Example 2. Dengue Virus and Dendritic Cells for the Treatment of Melanoma in a First Mouse Model

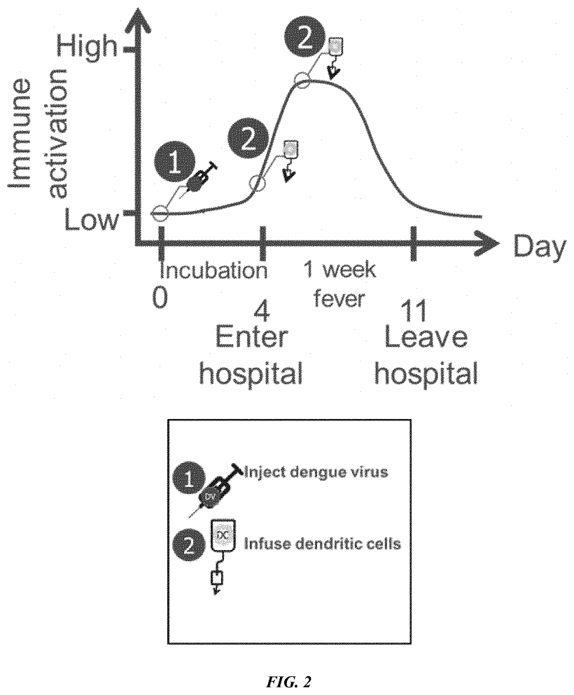

A mouse model assay was performed to observe results from combination targeting of cancer cells using a Dengue virus (DV) strain and tumor antigen primed dendritic cells (DCs). DV C57BL/6 mice were inoculated with 0.05 ml of Dengue virus (DEN-2 strain #1710) at 1×10⁶ or 1×10⁷ pfu/ml by injection in the base of tail. Recombinant murine IL-2 (Genzyme) and IFN-gamma (Sigma Pharmaceuticals) were administered by intravenous infusion at 2,000 (rIL-2) and 500 1U (rIFN-gamma) on days 5, 10, 15, and 20 following administration of Dengue virus (DEN-2 strain #1710, CDC database entry number 555, provided by Dr. Duane Gubler). Seven days after the Dengue virus administration, C57BL/6 mice were immunized with mouse DCs incubated with the 2 peptides separately and injected intravenously. Peptides were synthesized. The H-2b-restricted peptide from Ovalbumin (OVA-8), SIINFEKL (SEQ ID NO: 7), was used as a control. B16 melanoma-associated H-2b-restricted peptides derived from the antigens gp100/pme117 (EGSRNQDWL (SEQ ID NO: 1)) and from TRP-1/75 (TAYRYHLL (SEQ ID NO: 2)) were used to pulse murine DCs (see Example 1 for details). Two additional immunizations with DCs were given at 14-day intervals. Three days after the last DC infusion, mice were challenged with 5×10⁴ viable B16 melanoma cells intravenously in the lateral tail vein and then followed for survival, which was recorded as the percentage of surviving animals over time (in days) after tumor injection. Data was recorded from five or more mice/group (see Table 5 and FIG. 2).

TABLE 5

| | | | NO. OF | |
|---|---|---|---|---|
| | | | LUNG | |
| | | MOUSE | METAS- | |
| Condition | Group | ID | TASES | Mean |
| DV10⁶ pfu/ml + 2 × 10⁶ | 2 | II-2-1 | 55 | |
| DCs pulsed with gp100/TRP2 | | | | |
| DV10⁶ pfu/ml + 2 × 10⁶ | 2 | II-2-2 | 68 | |
| DCs pulsed with gp100/TRP2 | | | | |
| DV10⁶ pfu/ml + 2 × 10⁶ | 2 | II-2-3 | 57 | |
| DCs pulsed with gp100/TRP2 | | | | |
| DV10⁶ pfu/ml + 2 × 10⁶ | 2 | II-2-4 | 62 | |
| DCs pulsed with gp100/TRP2 | | | | |
| DV10⁶ pfu/ml + 2 × 10⁶ | 2 | II-2-5 | 52 | 58.8 |
| DCs pulsed with gp100/TRP2 | | | | |
| No DV + 2 × 10⁶ DCs | 1 | II-1-1 | 58 | |
| pulsed with gp100/TRP2 | | | | |
| No DV + 2 × 10⁶ D DCs C | 1 | II-1-2 | 62 | |
| pulsed with gp100/TRP2 | | | | |
| No DV + 2 × 10⁶ DCs | 1 | II-1-3 | 66 | |
| pulsed with gp100/TRP2 | | | | |
| No DV + 2 × 10⁶ DCs | 1 | II-1-4 | 72 | |
| pulsed with gp100/TRP2 | | | | |
| No DV + 2 × 10⁶ DCs | 1 | II-1-5 | 60 | 63.6 |
| pulsed with gp100/TRP2 | | | | |

The number of lung metastases observed in mice administered in Group 2 (Dengue Virus serotype 2 strain #1710 and tumor peptide primed DCs) was 7.5% lower than control mice in Group 1, administered the tumor peptide primed DCs without the Dengue virus.

### Example 3. Dengue Virus and Dendritic Cells for the Treatment of Melanoma in a Second Mouse Model

A mouse model assay was performed to observe results from combination targeting of cancer cells using a Dengue virus (DV) strain and tumor antigen primed DCs. Mice were administered cytokines to parallel the response to DV observed in humans.

Tumors were established in mice using the H-2b-restricted B16 murine melanoma cells line (ATCC #CRL-6322). Peptides (B16 melanoma associated H-2b-restricted peptides derived from antigens gp100/pme117 and from TRP-1/gp75) used for pulsing the dendritic cells were synthesized. Dendritic cells were generated from mouse bone marrow according to methods as described in Lutz et al. (J. Immunol. Methods 223:77-92, 1999).

On day 0, mice received 5×10⁴ viable B16 melanoma cells intravenously in the lateral tail vein to establish pulmonary metastases. On day 7, the mice were inoculated with 0.05 ml of Dengue virus (DEN-2 strain #1710, CDC database entry number 555) at 1×10⁶ or 1×10⁷ pfu/ml by injection in the base of tail. Recombinant murine IL-2 (Genzyme) and IFN-gamma (Sigma Pharmaceuticals) were administered by intravenous infusion at 2,000 1U (rIL-2) and 500 1U (rIFN-gamma) at 5-day intervals following administration of Dengue virus (DEN-2 strain #1710). On days 21, 35 and 49, the mouse DCs were incubated with the 2 peptides separately and injected intravenously in 2 sequential administrations on the same day to match the route and schedule of administration in subjects (see Example 2 for additional details). Control groups of mice received no Dengue virus or dendritic cells pulsed with H-²b-restricted peptide from ovalbumin (OVA-8), SIINFEKL (SEQ ID NO: 7). Treatment and control groups are shown in Table 6.

TABLE 6
Experimental groups for testing Dengue virus and DC effects on melanoma
metastasis to lung

| | |
|---|---|
| Dengue Virus | # of dendritic cells and type of peptide |

Group A

| | |
|---|---|
| 10⁶ pfu/ml | 10⁶ DCs pulsed with gp100/pmel17 (EGSRNQDWL)(SEQ ID NO: 1) |
| | 10⁶ DCs pulsed with TRP-1/gp75 (TAYRYHLL)(SEQ ID NO: 2) |
| Total | 2 × 10⁶ DCs pulsed with peptide/mouse |

Group B

| | |
|---|---|
| 10⁶ pfu/ml | 10⁷ DCs pulsed with gp100/pmel17 (EGSRNQDWL)(SEQ ID NO: 1) |
| | 10⁷ DCs pulsed with TRP-1/gp75 (TAYRYHLL)(SEQ ID NO: 2) |
| Total | 2 × 10⁷ DCs pulsed with peptide/mouse |

Group C - Control

| | |
|---|---|
| None | 10⁶ DCs pulsed with gp100/pmel17 (EGSRNQDWL)(SEQ ID NO: 1) |
| | 10⁶ DCs pulsed with TRP-1/gp75 (TAYRYHLL)(SEQ ID NO: 2) |
| Total | 2 × 10⁶ DCs pulsed with peptide/mouse |

Group D - Control

| | |
|---|---|
| 10⁶ pfu/ml | 10⁶ DCs pulsed with OVA (SIINFEKL)(SEQ ID NO: 7) |
| | 10⁶ DCs pulsed with OVA (SIINFEKL)(SEQ ID NO: 7) |
| Total | 2 × 10⁶ DCs pulsed with peptide/mouse |

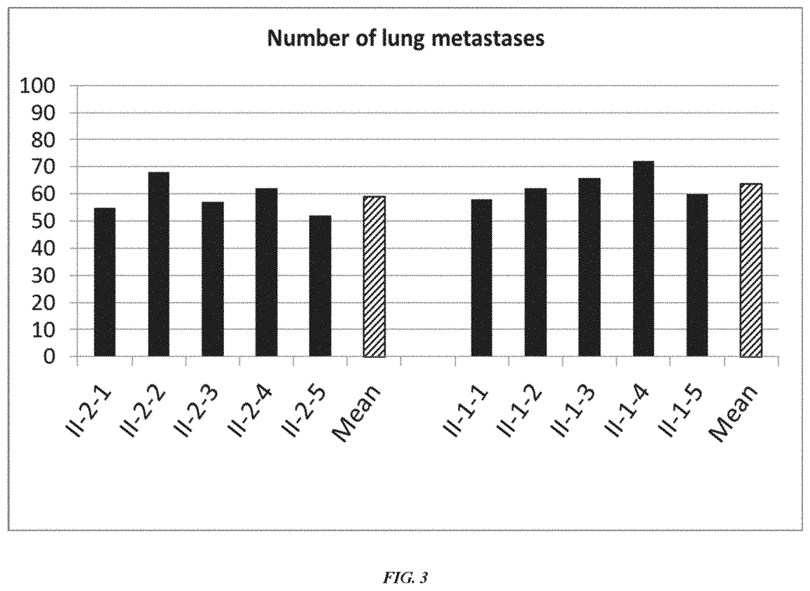

On day 90, animals were sacrificed and lung tumor colonies were counted. Pulmonary metastases were enumerated in a blinded, coded fashion after insufflation and fixation of the lungs with Fekette's solution. Data were reported as the mean number of metastases; four mice/group (see Table 7 and FIG. 3). Histopathology of the following major organ systems were performed: brain, heart, lungs, liver, kidneys, spleen and gonads (data not shown).

TABLE 7
Results for testing Dengue virus and DC
effects on melanoma metastasis to lung

| | | | NO. OF | |
|---|---|---|---|---|
| | | | LUNG | |
| | | MOUSE | METAS- | |
| Condition | Group | ID | TASES | Mean |
| DV10⁶ pfu/ml + 2 × 10⁶ | A | III-1-1 | 82 | |
| DC pulsed with gp100/TRP2 | | | | |
| DV10⁶ pfu/ml + 2 × 10⁶ | A | III-1-2 | 87 | |
| DC pulsed with gp100/TRP2 | | | | |
| DV10⁶ pfu/ml + 2 × 10⁶ | A | III-1-3 | 78 | |
| DC pulsed with gp100/TRP2 | | | | |
| DV10⁶ pfu/ml + 2 × 10⁶ | A | III-1-4 | 72 | |
| DC pulsed with gp100/TRP2 | | | | |
| | | | | 79.75 |
| DV10⁷ pfu/ml + 2 × 10⁶ | B | III-2-1 | 87 | |
| DC pulsed with gp100/TRP2 | | | | |
| DV10⁷ pfu/ml + 2 × 10⁶ | B | III-2-2 | 77 | |
| DC pulsed with gp100/TRP2 | | | | |
| DV10⁷ pfu/ml + 2 × 10⁶ | B | III-2-3 | 92 | |
| DC pulsed with gp100/TRP2 | | | | |
| DV10⁷ pfu/ml + 2 × 10⁶ | B | III-2-4 | 85 | |
| DC pulsed with gp100/TRP2 | | | | |
| | | | | 85.25 |
| No dengue virus + 2 × 10⁶ | C | III-3-1 | 97 | |
| DC pulsed with gp100/TRP2 | | | | |
| No dengue virus + 2 × 10⁶ | C | III-3-2 | 94 | |
| DC pulsed with gp100/TRP2 | | | | |
| No dengue virus + 2 × 10⁶ | C | III-3-3 | 88 | |
| DC pulsed with gp100/TRP2 | | | | |
| No dengue virus + 2 × 10⁶ | C | III-3-4 | 91 | |
| DC pulsed with gp100/TRP2 | | | | |
| | | | | 92.5 |
| DV10⁶ pfu/ml + 2 × 10⁶ | D | III-4-1 | 180 | |
| DC pulsed with OV | | | | |
| DV10⁶ pfu/ml + 2 × 10⁶ | D | III-4-2 | 174 | |
| DC pulsed with OV | | | | |
| DV10⁶ pfu/ml + 2 × 10⁶ | D | III-4-3 | 165 | |
| DC pulsed with OV | | | | |
| DV10⁶ pfu/ml + 2 × 10⁶ | D | III-4-4 | 177 | |
| DC pulsed with OV | | | | |
| | | | | 174 |

The number of lung metastases observed in mice in Group C (administered tumor antigen primed DCs and no virus) was 47% less than control Group D (administered DENV-2 #1710 and DCs exposed to a control peptide). The number of lung metastases observed in mice in Group A (administered DENV-2 #1710 and tumor antigen primed DCs) was 54% less than control Group D (administered DENV-2 #1710 and DCs exposed to a control peptide). The number of lung metastases observed in mice in Group B (administered DENV-2 #1710 and tumor antigen primed DCs) was 51% less than control Group D (administered DENV-2 #1710 and DCs exposed to a control peptide). The average reduction in Group A and B compared to Group D was 52.8%.

### Example 4. Manufacture and Screening of Dengue Virus

A Master Cell Bank with validated and certified cell lines from Vero (African Green Monkey Kidney Cells) was generated and tested for absence of any contaminants and adventitious organisms. Vero lines are used by the World Health Organizations to produce a variety of viral vaccines. Dengue virus was passaged in a validated Vero Line derived from the Master Cell Bank and established as a Working Cell Bank according to guidelines established by the FDA Center for Biologics (CBER). Two Dengue Virus Type 2 strains (DNV-2 #1584 and DENV-2 #1710) from initial seed stocks were added to the Vero Cells of the WCB at a MOI of 10-.

The first 4-ml overlay medium-containing 1% SeaKem LE agarose (FMC BioProducts, Rockland, Me.) in nutrient medium (0.165% lactalbumin hydrolysate [Difco Laboratories, Detroit, Mich.]), 0.033% yeast extract [Difco], Earle's balanced salt solution, 25 mg of gentamicin sulfate [BioWhittaker, Walkersville, Md.] and 1.0 mg of amphotericin B [Fungizone; E. R. Squibb & Sons, Princeton, N.J.], per liter and 2% FBS)—was added after adsorption of the 200-ml virus inoculum for 1.5 h at 37° C. Following incubation at 37° C. for 7 days, a second 2-ml overlay containing additional 80 mg of neutral red vital stain (GIBCO-BRL, Gaithersburg, Md.) per ml was added. Plaques were counted 8 to 11 days after infection.

A plaque assay on final virus cultures was performed. The titer of DNV-2 #1584 was approximately 5E+06 PFU/ml, and the titer of DENV-2 #1710 was 3.5E+06 pfu/mL as estimated from plaque assays. Dengue virus 2 (DNV-2; #1584) from ATCC showed a clear cytopathic effect in Vero cells 5 days post infection, whereas Vero cells appears to have a morphology change 11 days post infection of the blind passage #2 (#1710 virus). (Data not shown.) DENV-2 #1710 virus was shown to be far less cytopathic than the DNV-2 #1584 strain.

### Example 5. Cancer Killing Assay with Pulsed DC, with and without DV

In a control arm, normal human tumor infiltrating lymphocytes (TILs) were directly applied to human melanoma FEMX cells. T-cell receptors were matched to FEMX melanoma cell line via HLA A2.1+. In a treatment arm, human TILs were exposed to DV supematants containing interferons and interleukins. Exposed TILs+DV supernatants were placed in culture with FEMX tumor cells. Both arms were left to kill cancer cells for 4 hours at a ratio of 5-to-1T-cell to tumor cell (100,000 cells to 20,000 cells). Surviving tumor cells were then counted as % of starting cells by flow cytometry. Results, shown in Table 8, demonstrate that DV induces 35% additional cancer cell killing beyond the pulsed DC anti-cancer response.

TABLE 8
DV enhancement of pulsed DC anti-cancer activity

| | | % FL2-A+ |
|---|---|---|
| | % FL2-A− | (% Apoptotic Cells) |
| CTL | 86.1% | 13.9% |
| CTL + DV Sups | 81.2% | 18.8% |

### Example 6. Human Dendritic Cell Isolation and Pulsing with Melanoma Lysate Antigens

The following example demonstrates generation of a highly pure CD11a+ mature DC population expressing high levels of human IL-12p70 from pure, isolated CD14+ monocytes, as well as priming of the DC with melanoma cell lysate, the entire process being completed in less than one week. Cells were cultured on hard plastic plates and not exposed to soft plastic bags.

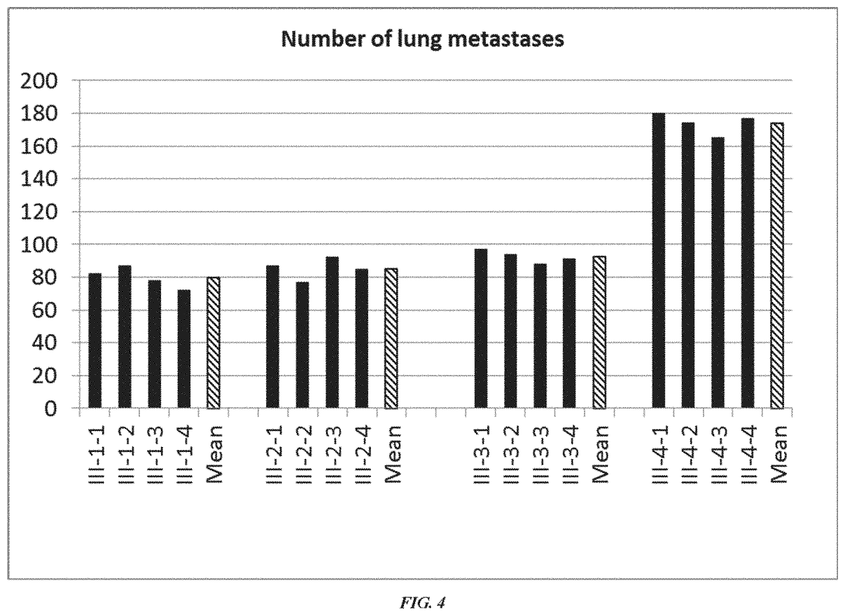

CD14+ monocytes were isolated and analyzed for expression of CD14, CD15, CD45 and 7AAD. Post-prodigy run, 90.25% of input cells were CD14+(see FIG. 4). CD14+ cells were treated with GM-CSF and IL-4 24 hours post plating to generate immature dendritic cells. FEMX melanoma cells from Providence Cancer Institute arrived on the day of the prodigy run and were re-suspended, counted and plated. Melanoma cells were than treated with a calcium hypochlorite solution. Alternatively, cells were treated with sodium chlorite solution. The melanoma cell lysate was added to the immature DC, and maturing agents IFN-gamma (1000U/mL), R848 (5 μg/mL), LPS (10 ng/mL), and CD40L (1 microgram/mL) were added. In terms of timing, LPS was administered early, and IFN-gamma and were R848 was administered subsequently. CD40L was administered last in the maturation process.

Supernatant from mature DCs were collected for mycoplasma and endotoxin testing 22 hours after pulsing with melanoma cell lysate and 18 hours after addition of maturing agents. No organisms or growth were observed. In addition, ELISA was used to test for IL-12p70 levels, an indicator of the potency of the DCs using 13 dilutions of the DC culture medium supernatant. The concentration of IL-12p70 was 19+/−4 ng/mL, as opposed to the industry standard of 4-6 ng/mL. FIG. 5 shows DC IL-12p70 production relative to that of several comparators. These comparators methods include exposing cells to soft plastic bags, lysing cells with solutions other than a chlorite solution, and do not use the combination of LPS, IFN gamma and R848 to mature cells. Repeated experiments using HOCL solution instead of HOCL powder for the lysis step provided concentrations of IL-12p70 as high as 29 ng/mL.

Cells were further frozen and then thawed at 4° C. to test cell counts and viability after freezing and thawing. These were measured at approximately 16h, 18h, 20h and 22h after beginning of thaw. An extra harvest of non-pulsed DCs were tested in a cryopreservation study, and showed viability at 80%, which is greater than an industry standard of 70% viability. Pre-cryopreservation viability ranged from 85-89%.

### Example 7. Inducing Cytokines in Human White Blood Cells with Dengue Virus

Human white blood cells (WBC), including monocytes, dendritic cells and T lymphocytes, were infected with either mock virus or Dengue virus (DENV-2 #1710) at three different multiplicities of infection (MOI), MOI of 0.1, MOI of 0.5 and MOI of 2 at time=0. Levels (pg/mL) of various cytokines were measured at 48h, 72h and 96h, post-infection. Treatments were performed in triplicate. Results are shown for each time point in Tables 9-12. (M=mock. 0.1, 0.5 and 2 are MOI). Triplicate average of changes between mock and Dengue virus at the tested MOIs was calculated and shown as a percentage in Table 9. This experiment and repeated experiments demonstrate DV induces a 70%-4000% increase in cytokines like GM-CSF, IL-7 and IP-10, as compared to mock virus.

TABLE 9
Cytokine levels produced by human WBC, 48 h post- Dengue
virus infection, measured in picograms/milliliter

| | M | M | M | 0.1 | 0.1 | 0.1 |
|---|---|---|---|---|---|---|
| IL-1b | 15 | 6 | 6 | 6 | 6 | 6 |
| IL-10 | 4 | 4 | 4 | 4 | 4 | 4 |
| IL-13 | 11 | 11 | 11 | 11 | 11 | 11 |
| IL-6 | 12 | 7 | 9 | 941 | 874 | 788 |
| IL-12 | 19 | 12 | 13 | 14 | 15 | 15 |
| Rantes | 12 | 11 | 11 | 14 | 16 | 18 |
| CCL-11 | 3 | 3 | 3 | 3 | 3 | 3 |
| IL-17 | 18 | 18 | 18 | 18 | 18 | 18 |
| MIP-1a | 123 | 110 | 109 | 183 | 166 | 219 |
| GM-CSF | 5 | 5 | 5 | 5 | 5 | 5 |
| MIP-1b | 83 | 78 | 82 | 123 | 111 | 118 |
| MCP-1 | 1.77e+03 | 1.48e+03 | 1.87e+03 | 12.6e+03 | 10.4e+03 | 9.95e+03 |
| IL-15 | 33 | 33 | 33 | 33 | 33 | 33 |
| IL-5 | 8 | 8 | 8 | 8 | 8 | 8 |
| IFN-g | 5 | 5 | 5 | 6 | 6 | 6 |
| IFN-a | 16 | 12 | 12 | 37 | 35 | 33 |
| IL-1Ra | 3.37e+03 | 2.84e+03 | 3.59e+03 | 4.99e+03 | 4.39e+03 | 4.30e+03 |
| TNF-a | 6 | 6 | 6 | 8 | 8 | 8 |
| IL-2 | 9 | 9 | 9 | 9 | 9 | 9 |
| IL-7 | 16 | 8 | 11 | 31 | 27 | 26 |
| IP-10 | 4 | 4 | 4 | 23 | 15 | 18 |
| IL-2R | 31 | 31 | 31 | 54 | 47 | 52 |
| MIG | 38 | 32 | 39 | 29 | 26 | 26 |
| IL-4 | 23 | 23 | 23 | 23 | 23 | 23 |
| IL-8 | 17.8e+03 | 17.8e+03 | 17.8e+03 | 17.8e+03 | 17.8e+03 | 17.8e+03 |

| | 0.5 | 0.5 | 0.5 | 2 | 2 | 2 |
|---|---|---|---|---|---|---|
| IL-1b | 6 | 6 | 6 | 7 | 7 | 7 |
| IL-10 | 4 | 4 | 5 | 5 | 4 | 4 |
| IL-13 | 11 | 11 | 11 | 11 | 11 | 11 |
| IL-6 | 8.08e+03 | 8.64e+03 | 10.0e+03 | 11.2e+03 | 11.2e+03 | 11.2e+03 |
| IL-12 | 17 | 20 | 19 | 28 | 25 | 25 |
| Rantes | 32 | 56 | 64 | 152 | 135 | 148 |
| CCL-11 | 3 | 3 | 3 | 3 | 3 | 3 |
| IL-17 | 18 | 18 | 18 | 18 | 18 | 18 |
| MIP-1a | 212 | 309 | 328 | 261 | 264 | 259 |
| GM-CSF | 5 | 6 | 7 | 22 | 20 | 21 |
| MIP-1b | 145 | 152 | 142 | 163 | 149 | 155 |
| MCP-1 | 21.8e+03 | 23.4e+03 | 24.2e+03 | 32.0e+03 | 32.0e+03 | 32.0e+03 |
| IL-15 | 33 | 33 | 33 | 68 | 63 | 60 |
| IL-5 | 16 | 18 | 18 | 21 | 21 | 20 |
| IFN-g | 8 | 8 | 8 | 10 | 9 | 10 |
| IFN-a | 47 | 50 | 47 | 67 | 68 | 71 |
| IL-1Ra | 4.55e+03 | 4.88e+03 | 5.14e+03 | 4.13e+03 | 3.42e+03 | 3.82e+03 |
| TNF-a | 16 | 13 | 11 | 21 | 21 | 19 |
| IL-2 | 9 | 9 | 9 | 9 | 9 | 9 |
| IL-7 | 51 | 49 | 47 | 53 | 55 | 54 |
| IP-10 | 39 | 46 | 39 | 218 | 128 | 147 |
| IL-2R | 57 | 69 | 69 | 79 | 76 | 79 |
| MIG | 26 | 31 | 28 | 23 | 22 | 27 |
| IL-4 | 27 | 27 | 27 | 30 | 29 | 30 |
| IL-8 | 17.8e+03 | 17.8e+03 | 17.8e+03 | 17.8e+03 | 17.8e+03 | 17.8e+03 |

TABLE 10
Cytokine levels produced by human WBC, 72 h post- Dengue
virus infection, measured in picograms/milliliter

| | M | M | M | 0.1 | 0.1 | 0.1 |
|---|---|---|---|---|---|---|
| IL-1b | 6 | 6 | 6 | 6 | 6 | 6 |
| IL-10 | 4 | 4 | 4 | 4 | 4 | 4 |
| IL-13 | 11 | 11 | 11 | 11 | 11 | 11 |
| IL-6 | 7 | 7 | 7 | 637 | 690 | 737 |
| IL-12 | 12 | 11 | 11 | 12 | 12 | 14 |
| Rantes | 11 | 11 | 11 | 11 | 11 | 11 |
| CCL-11 | 3 | 3 | 3 | 3 | 3 | 3 |
| IL-17 | 18 | 18 | 18 | 18 | 18 | 18 |
| MIP-1a | 96 | 88 | 88 | 84 | 97 | 118 |
| GM-CSF | 5 | 5 | 5 | 5 | 5 | 5 |
| MIP-1b | 83 | 78 | 80 | 85 | 90 | 101 |
| MCP-1 | 5.51e+03 | 5.02e+03 | 4.87e+03 | 21.5e+03 | 22.4e+03 | 21.7e+03 |
| IL-15 | 33 | 33 | 33 | 33 | 33 | 33 |
| IL-5 | 8 | 8 | 8 | 8 | 8 | 8 |
| IFN-g | 5 | 5 | 5 | 6 | 6 | 6 |
| IFN-a | 26 | 23 | 24 | 43 | 46 | 46 |
| IL-1Ra | 6.30e+03 | 5.97e+03 | 6.02e+03 | 6.36e+03 | 6.89e+03 | 6.36e+03 |
| TNF-a | 6 | 6 | 6 | 6 | 6 | 6 |
| IL-2 | 9 | 9 | 9 | 9 | 9 | 9 |
| IL-7 | 8 | 8 | 8 | 23 | 25 | 21 |
| IP-10 | 4 | 4 | 4 | 18 | 14 | 17 |
| IL-2R | 31 | 28 | 20 | 42 | 44 | 42 |
| MIG | 40 | 35 | 35 | 32 | 28 | 27 |
| IL-4 | 23 | 23 | 23 | 23 | 23 | 23 |
| IL-8 | 17.8e+03 | 17.8e+03 | 17.8e+03 | 17.8e+03 | 17.8e+03 | 17.8e+03 |

| | 0.5 | 0.5 | 0.5 | 2 | 2 | 2 |
|---|---|---|---|---|---|---|
| IL-1b | 6 | 6 | 6 | 6 | 7 | 7 |
| IL-10 | 5 | 5 | 4 | 4 | 5 | 5 |
| IL-13 | 11 | 11 | 11 | 11 | 11 | 11 |
| IL-6 | 5518 | 8803 | 6841 | 11.2e+03 | 11.2e+03 | 11.2e+03 |
| IL-12 | 15 | 17 | 16 | 17 | 20 | 22 |
| Rantes | 11 | 16 | 15 | 21 | 88 | 68 |
| CCL-11 | 3 | 3 | 3 | 3 | 3 | 3 |
| IL-17 | 18 | 18 | 18 | 18 | 18 | 18 |
| MIP-1a | 91 | 118 | 106 | 54 | 133 | 87 |
| GM-CSF | 5 | 5 | 5 | 8 | 15 | 15 |
| MIP-1b | 104 | 112 | 101 | 84 | 98 | 101 |
| MCP-1 | 32.0e+03 | 32.0e+03 | 32.0e+03 | 32.0e+03 | 32.0e+03 | 32.0e+03 |
| IL-15 | 33 | 33 | 33 | 33 | 38 | 67 |
| IL-5 | 14 | 15 | 14 | 17 | 19 | 20 |
| IFN-g | 8 | 8 | 7 | 6 | 8 | 8 |
| IFN-a | 62 | 56 | 52 | 61 | 66 | 67 |
| IL-1Ra | 6.90e+03 | 6.76e+03 | 6.01e+03 | 4.33e+03 | 3.89e+03 | 4.39e+03 |
| TNF-a | 6 | 6 | 6 | 6 | 6 | 6 |
| IL-2 | 9 | 9 | 9 | 9 | 9 | 9 |
| IL-7 | 42 | 40 | 40 | 45 | 50 | 48 |
| IP-10 | 42 | 38 | 38 | 104 | 143 | 169 |
| IL-2R | 42 | 47 | 47 | 44 | 56 | 60 |
| MIG | 27 | 25 | 22 | 24 | 19 | 25 |
| IL-4 | 27 | 25 | 24 | 26 | 27 | 29 |
| IL-8 | 17.8e+03 | 17.8e+03 | 17.8e+03 | 17.8e+03 | 17.8e+03 | 17.8e+03 |

TABLE 11
Cytokine levels produced by human WBC, 96 h post- Dengue
virus infection, measured in picograms/milliliter

| | M | M | M | 0.1 | 0.1 | 0.1 |
|---|---|---|---|---|---|---|
| IL-1b | 6 | 6 | 6 | 6 | 6 | 6 |
| IL-10 | 4 | 4 | 4 | 5 | 4 | 4 |
| IL-13 | 11 | 11 | 11 | 11 | 11 | 11 |
| IL-6 | 9 | 9 | 9 | 834 | 734 | 771 |
| IL-12 | 14 | 13 | 13 | 16 | 14 | 14 |
| Rantes | 11 | 11 | 11 | 11 | 11 | 11 |
| CCL-11 | 3 | 3 | 3 | 3 | 3 | 3 |
| IL-17 | 18 | 18 | 18 | 18 | 18 | 18 |
| MIP-1a | 98 | 89 | 119 | 73 | 103 | 122 |
| GM-CSF | 5 | 5 | 5 | 5 | 5 | 5 |
| MIP-1b | 82 | 78 | 99 | 63 | 89 | 99 |
| MCP-1 | 8.19e+03 | 7.61e+03 | 7.10e+03 | 32.0e+03 | 25.3e+03 | 25.6e+03 |
| IL-15 | 33 | 33 | 33 | 33 | 33 | 33 |
| IL-5 | 8 | 8 | 8 | 8 | 8 | 8 |
| IFN-g | 6 | 6 | 7 | 8 | 7 | 6 |
| IFN-a | 27 | 29 | 27 | 52 | 47 | 44 |
| IL-1Ra | 10.9e+03 | 10.9e+03 | 10.2e+03 | 11.0e+03 | 9.57e+03 | 9.56e+03 |
| TNF-a | 6 | 6 | 6 | 6 | 6 | 6 |
| IL-2 | 9 | 9 | 9 | 9 | 9 | 9 |
| IL-7 | 8 | 8 | 8 | 21 | 18 | 14 |
| IP-10 | 4 | 4 | 4 | 29 | 11 | 11 |
| IL-2R | 25 | 23 | 28 | 39 | 36 | 42 |
| MIG | 39 | 40 | 39 | 39 | 24 | 26 |
| IL-4 | 23 | 23 | 23 | 23 | 23 | 23 |
| IL-8 | 17.8e+03 | 17.8e+03 | 17.8e+03 | 17.8e+03 | 17.8e+03 | 17.8e+03 |

| | 0.5 | 0.5 | 0.5 | 2 | 2 | 2 |
|---|---|---|---|---|---|---|
| IL-1b | 6 | 6 | 7 | 7 | 6 | 7 |
| IL-10 | 5 | 6 | 6 | 5 | 5 | 5 |
| IL-13 | 11 | 11 | 11 | 11 | 11 | 11 |
| IL-6 | 7026 | 7.47e+03 | 7.65e+03 | 11.2e+03 | 11.2e+03 | 11.2e+03 |
| IL-12 | 16 | 14 | 16 | 16 | 20 | 20 |
| Rantes | 11 | 11 | 11 | 37 | 70 | 68 |
| CCL-11 | 3 | 3 | 3 | 3 | 3 | 3 |
| IL-17 | 18 | 18 | 18 | 18 | 18 | 18 |
| MIP-1a | 79 | 77 | 85 | 60 | 108 | 106 |
| GM-CSF | 5 | 5 | 5 | 12 | 14 | 15 |
| MIP-1b | 85 | 83 | 89 | 67 | 72 | 76 |
| MCP-1 | 32.0e+03 | 32.0e+03 | 32.0e+03 | 32.0e+03 | 32.0e+03 | 32.0e+03 |
| IL-15 | 33 | 33 | 33 | 49 | 43 | 52 |
| IL-5 | 15 | 16 | 16 | 20 | 19 | 18 |
| IFN-g | 7 | 7 | 7 | 7 | 7 | 7 |
| IFN-a | 56 | 58 | 65 | 64 | 64 | 67 |
| IL-1Ra | 7.63e+03 | 7.80e+03 | 8.27e+03 | 5.49e+03 | 4.22e+03 | 4.45e+03 |
| TNF-a | 6 | 6 | 6 | 6 | 6 | 6 |
| IL-2 | 9 | 9 | 9 | 9 | 9 | 9 |
| IL-7 | 33 | 37 | 48 | 50 | 45 | 44 |
| IP-10 | 29 | 28 | 33 | 134 | 101 | 104 |
| IL-2R | 39 | 42 | 59 | 52 | 49 | 57 |
| MIG | 19 | 22 | 24 | 20 | 17 | 18 |
| IL-4 | 25 | 24 | 25 | 27 | 27 | 28 |
| IL-8 | 17.8e+03 | 17.8e+03 | 17.8e+03 | 17.8e+03 | 17.8e+03 | 17.8e+03 |

TABLE 12
Relative changes in WBC cytokine levels between mock and Dengue infections

| | MOI 0.1 | | | MOI 0.5 | |
|---|---|---|---|---|---|
| | 48 h | 72 h | 96 h | 48 h | 72 h |
| IL-1b | −33% | 0% | 0% | −33% | 0% |
| IL-10 | 0% | 0% | 8% | 8% | 17% |
| IL-13 | 0% | 0% | 0% | 0% | 0% |
| IL-6 | 9.20E+03% | 9.73E+03% | 8.56E+03% | 95.4E+03% | 10.1E+04% |
| IL-12 | 0% | 12% | 10% | 27% | 41% |
| Rantes | 41% | 0% | 0% | 347% | 27% |
| CCL-11 | 0% | 0% | 0% | 0% | 0% |
| IL-17 | 0% | 0% | 0% | 0% | 0% |
| MIP-1a | 66% | 10% | −3% | 148% | 16% |
| GM-CSF | 0% | 0% | 0% | 20% | 0% |
| MIP-1b | 45% | 15% | −3% | 81% | 32% |
| MCP-1 | 543% | 325% | 262% | 1255% | 523% |
| IL-15 | 0% | 0% | 0% | 0% | 0% |
| IL-5 | 0% | 0% | 0% | 117% | 79% |
| IFN-g | 20% | 20% | 11% | 60% | 53% |
| IFN-a | 163% | 85% | 72% | 260% | 133% |
| IL-1Ra | 39% | 7% | −6% | 49% | 7% |
| TNF-a | 33% | 0% | 0% | 122% | 0% |
| IL-2 | 0% | 0% | 0% | 0% | 0% |
| IL-7 | 140% | 188% | 121% | 320% | 408% |
| IP-10 | 367% | 308% | 325% | 933% | 883% |
| IL-2R | 65% | 62% | 54% | 110% | 72% |
| MIG | −26% | −21% | −25% | −22% | −33% |
| IL-4 | 0% | 0% | 0% | 17% | 10% |
| IL-8 | 0% | 0% | 0% | 0% | 0% |

| | MOI 0.5 | MOI 2 | | |
|---|---|---|---|---|
| | 96 h | 48 h | 72 h | 96 h |
| IL-1b | 6% | −22% | 11% | 11% |
| IL-10 | 42% | 8% | 17% | 25% |
| IL-13 | 0% | 0% | 0% | 0% |
| IL-6 | 8.19E+03% | 12.02E+04% | 16.04E+04% | 12.46E+04% |
| IL-12 | 15% | 77% | 74% | 40% |
| Rantes | 0% | 1179% | 436% | 430% |
| CCL-11 | 0% | 0% | 0% | 0% |
| IL-17 | 0% | 0% | 0% | 0% |
| MIP-1a | −21% | 129% | 1% | −10% |
| GM-CSF | 0% | 320% | 153% | 173% |
| MIP-1b | −1% | 92% | 17% | −17% |
| MCP-1 | 319% | 1774% | 523% | 319% |
| IL-15 | 0% | 93% | 39% | 45% |
| IL-5 | 96% | 158% | 133% | 138% |
| IFN-g | 11% | 93% | 47% | 11% |
| IFN-a | 116% | 415% | 166% | 135% |
| IL-1Ra | −26% | 16% | −31% | −56% |
| TNF-a | 0% | 239% | 0% | 0% |
| IL-2 | 0% | 0% | 0% | 0% |
| IL-7 | 392% | 363% | 496% | 479% |
| IP-10 | 650% | 4008% | 3367% | 2725% |
| IL-2R | 84% | 152% | 103% | 108% |
| MIG | −45% | −34% | −38% | −53% |
| IL-4 | 7% | 29% | 19% | 19% |
| IL-8 | 0% | 0% | 0% | 0% |

### Example 8. Additional Virus Manufacturing Protocols

In addition to methods of Example 4, both Vero and FRhL cells are infected using dilutions of the supernatant from blind passage #2, DENV-2 #1710, DNV-2 #1584, and 45AZ5 respectively. In order to increase the detection sensitivity, an immunofluorescence staining is developed to detect virus in the cells infected with supernatant from blind passage #2.

Ultracentrifugation is used to concentrate virus when necessary. Following confirmation of virus titer, final product is filtered to remove any cellular debris, tested for absence of any adventitious organisms, and upon final lot released, bottled in 5 ml bottles, and stored at 4° C. until ready for shipment and administration.

### Example 9. Collection of PBMC from Donors

Donors (either autologous or HLA-matched allogenic) have a leukapheresis procedure performed at a facility with trained personnel and proper equipment. After the apheresis is complete, the red cells, platelets, and plasma proteins are returned to the donor. The apheresis product is tested at the site (Gram Stain test and Limulus Amoeba Lysis [LAL]) for presence of bacterial contamination. After passing, the collection container (with small testing sample container attached), is barcoded with donor-specific information and placed in an approved shipping container conforming to both FDA and DOT regulations for storage and shipping of non-infectious biological materials. The shipping container is packaged with a cooling element (e.g., solid CO2, Liquid N2), and temperature monitors. The shipping container is a hard plastic flask. A courier transports the container within 24 hours to the GMP manufacturing facility.

### Example 10. Manufacture and Use of Dendritic Cells Pulsed with Tumor Antigens

Monocytes are separated from other collected white blood cells (e.g., T cells. B cells, NK cells, eosinophils and basophils). This is accomplished with immuno-magnetic selection or, alternatively, by adherence properties. Immuno-magnetic selection involves pouring the white blood cells into a sterile plastic column with plastic beads coated with antibodies for immune cell CD surface proteins: (CD4/CD8/CD56, etc.).

An example of immunomagnetic selection is the EasySep Monocyte Enrichment kit available from Stem Cell Technologies (Vancouver, B.C, Canada, www.stemcell.com). To use the EasySep kit, the apheresis product is suspended in sterile PBS and poured into the EasySep plastic column containing Tetrameric antibody complexes with murine antibodies for: human CD2, CD3, CD16, CD19, CD20, CD56, CD66b, CD123, and Glycophorin A. After incubation for 10 minutes, EasySep magnetic particles are added. The cells adhering to the beads removed an electromagnet sorting. The magnet is inverted, and the desired cell fraction (monocytes), is poured into a sterile polystyrene flask for additional processing. Alternately, in a positive adherence selection assay, magnetic beads coated with CD1+/CD14+ antibodies is mixed with monocytes, a magnet is placed against the column, and non-binding cells are flushed out of the column with PBS solution. The monocytes are then washed off the beads. In positive adherence selection, the properties of monocytes to stick to certain surfaces are used to separate them by running the apheresis product down a slanted column.

Alternatively, bone marrow cells are depleted for lymphocytes and MHC Class positive cells by Fluorescent Activated Cell Sorting (FACS) with monoclonal antibodies for CD3, CD4, and CD8. Remaining cells are cultured overnight at 37° C. in a 5% CO₂ atmosphere in a basal cell culture medium supplemented with human AB serum. Human AB serum is chosen because it grows cells at a faster rate than other serum types, and serum free media produces DCs with much lower T-cell stimulation capability. After 24 hours, the cells are replated and cultured in the presence of Granulocyte-Macrophage Colony Stimulation Factor (GM-CSF), and recombinant IL-4 at 900 U/ml. After 3 to 4 days, media to be exchanged for fresh cytokine media.

Alternatively, dermal dendritic cells (DDCs) are prepared using the following methods: Keratomes from healthy human volunteers are incubated in a solution of the bacterial proteases Dispase type 2 at a final concentration of 1.2 U/ml in RPMI 1640 for 1 hour at 37° C. After the incubation period, epidermis and dermis are easily separated. Epidermal and dermal sheets are then cut into small (1-10 mm) pieces after several washing with PBS, and placed in RPMI 1640 supplemented with 10% Fetal Bovine Serum (FBS), and placed in 10-cm tissue culture plates. After 2-3 days, pieces of tissue are removed, and the medium collected. Cells migrating out of the tissue sections into the medium are spun down, resuspended in 1-2 ml fresh medium and stained with trypan blue. Further enrichment is achieved by separation on a metrizamide gradient. Cells are layered onto 3-ml columns of hypertonic 14.5% metrizamide and sedimented at 650 g for 10 minutes at room temperature. Low density interphase cells are collected and washed in two successively less hypertonic washes (RPMI 1640 with 10% FBS and 40 mM NaCl) to return cells to isotonicity.

When the monocytes are collected, they may number only a few thousand. The recombinant human growth factors rhulnterleukin-4 (IL-4), and rhuGranulocyte-Macrophage-Colony-Stimulation Factor (GM-CSF), are used in a multi-step protocol to accomplish the expansion of DC numbers to the range of 50 million. After the addition of IL-4 and GM-CSF, cells are assessed for and expansion in number and the development of mature-DC markers: (CD11⁺, CD80⁺, CD83+), as well as increased expression of both Class I (for presentation of short peptides to CD8⁺, and Class II MHC complexes (for presentation of longer peptides to CD4+Helper-Inducer T lymphocytes). After approximately 3-4 days, the number of mature DCs will be measured. For example, the monocyte-enriched fraction is placed in Nuclon-coated Cell Factory (Thermoscientific), with serum-free DC media (CellGro, Inc.), supplemented with GMP-2% human AB serum, 500 IU/ml (approximately 50 ng/ml) rhuIL-4 (CellGenix), with 500 IU/ml (approximately 50 ng/ml) rhuGM-CSF (CellGenix), added after the first 24 hours. Final product is approximately 1 L of total media volume. After about 72 hours of culture, a population of immature DCs are assessed for the following markers: CD1⁺CD11⁺CD14⁺.

### Example 11. Pulsing Dendritic Cells

A variety of tumor antigen sources are used for high-quality DCs: peptides, lysate from autologous tumors, whole tumor cells, and RNA coding for specific tumor antigens. An excisional biopsy or blood sample containing leukemic or lymphoma cells is obtained by surgery or blood draw followed by a magnetic selection to obtain leukemia/lymphoma cells. Once the tumor cells are obtained, they are barcoded and shipped in approved containers similar to those described for apheresis previously to the GMP facility. Samples may be frozen at −70° C. after passing bacterial contamination tests.

Whole autologous tumor cell lysate is prepared by several methods. To prepare the lysate, the tumor sample may be rewarmed to approximately 35° C. using a water bath or other procedure. The development of automated cell processors like the Miltenyi GentleMACS system allows the sample to be manually minced, suspended in PBS solution, then a pre-selected tissue-specific software-controlled rotor system separates the tumor cells. Cells are added to an enzyme mixture before being transferred to the Miltenyi GentleMACS dissociator. The single-cell suspension can be membrane-lysed with minimal damage to tumor peptides, using a hypochlorite solution, which will kill any residual tumor cells, neutralize dTH2 cytokines an increase immunogenicity for superior CTL affinity, avidity and activation. After adding hypochlorite, culture plates are incubated at 37 degrees Celsius, 5% CO₂, for 1 hour, with gentle manual agitation at 30 min to disperse hypochlorite. Cells are washed two time to neutralize the lysis reaction (e.g., with HBSS). Hypochlorite-treated cells may be subjected to subsequent freeze-thaw cycles. Alternatively, the sample does not separate the tumor cells. Instead the sample is left to contain tumor cells and supporting cells (e.g., cells from the tumor microenvironment). Cells are lysed with calcium hypochlorite to eliminate red blood cells and produce apoptotic and necrotic bodies without destroying peptides needed for CTL induction.

Lysate from the GentleMACS is added on the third day of immature DCs production. Immature DCs are co-cultured with tumor lysate for about 16 hours. The final step is maturation with an inflammatory signal. Clinical-Grade LPS (60 EU/ml) (R & D Invivogen), and Interferon-gamma (2000 IU/ml, approximately 100 ng/ml) (R&D Systems) are added to the flask and incubated for approximately 12 hours to mature the pulsed DC. After exposure to LPS, the DCs are assessed for up-regulation of CD80/CD83⁺ activation markers, and increase production of IL-12p70. In process testing at this stage includes sterility (as previously described), viability (% viable cells by Trypan Blue dye exclusion), and specificity (% DC measured by CD11c flow cytometry).

After final sterility, specificity, and viability testing, the DCs are transferred to hard plastic containers suitable for freezing at −130° C. in vapor phase N2, storage up to 1 year, and shipping to the clinic for use. The containers are shipped frozen overnight, then rewarmed to 37° C. in a dry bath before intravenous administration with a 0.9% NaCl solution concurrent over 30 minutes.

### Example 12. Combination Delivery for Treatment of Cancer

Administration of the Dengue Virus is similar to that of other viral vaccine injections. A subject has an area of skin in the shoulder (deltoid) region cleaned with alcohol, then 0.5 ml of the virus is injected under the skin to mimic a mosquito bite. Once the subject has a fever the reaches 38.5° C., after 2-3 days from DV injection, the subject is infused by intralymphatic microcatheter with pulsed (primed) dendritic cells. Injections are repeated until the subject is negative for disease. The DV strain in this example is DENV-1 #45AZ5 or DENV-2 #1710. DC infusions use cells as manufactured in Example 6.

### Example 13. Dengue Virus Cytotoxicity Analysis in Cancer Cell Lines

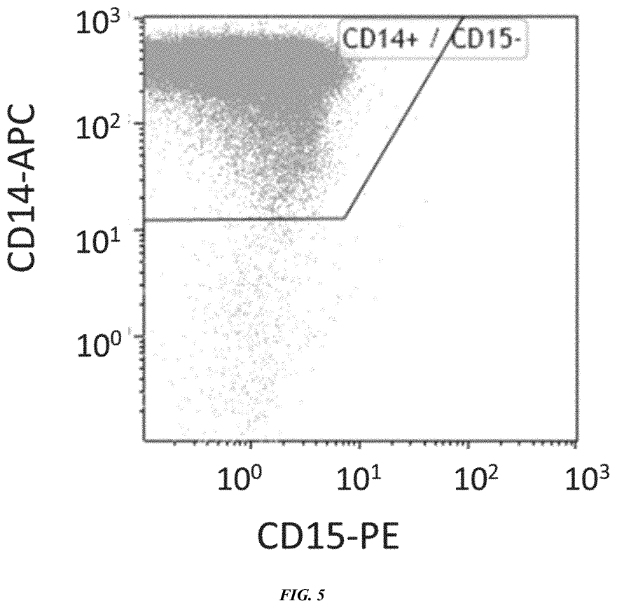

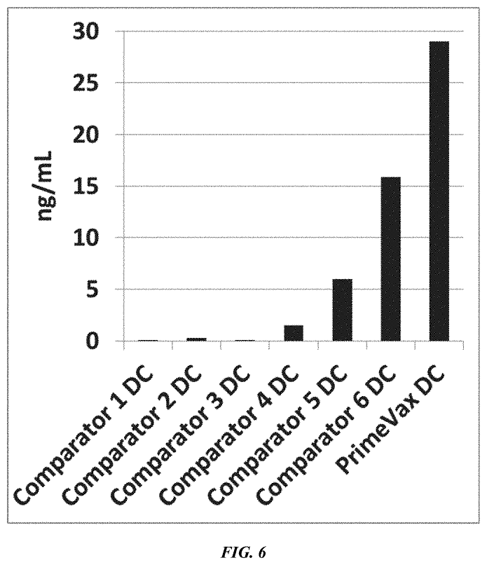

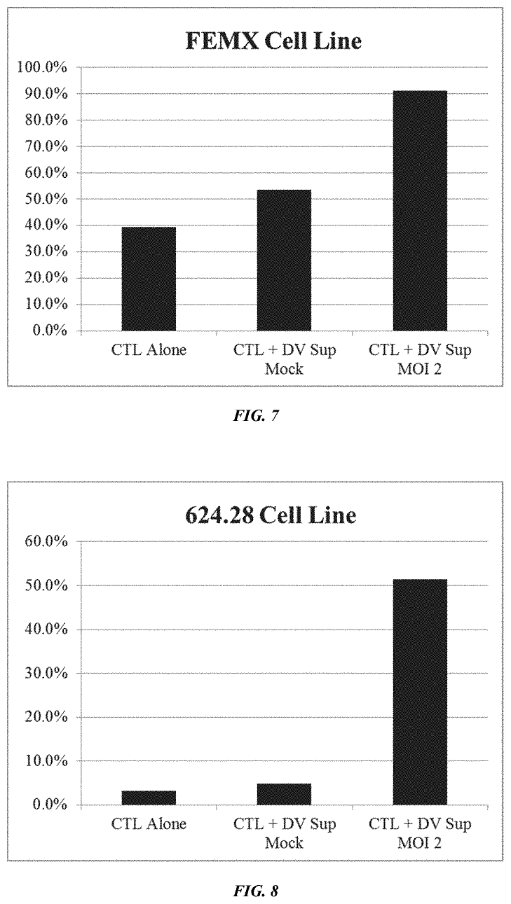

Two different cancer cell lines, FEMX and 624.28, were each separately co-cultured with CTL, either in the presence or absence of DV supernatant (MOI 2) for six hours, and cell death was quantified for each set of conditions using an LDH release assay. DV supernatant was obtained after infecting WBC with Dengue Virus in a method as described in Example 7. DV supernatant nearly doubled the ability of CTL to kill FEMX cells: 51% of FEMX cells were killed by CTL in the presence of mock supernatant and 91% of FEMX cells were killed by CTL in the presence of DV supernatant. DV supernatant dramatically increased the CTL's ability to kill 624.28 cells: 5% of 624.28 cells were killed by CTL in the presence of mock supernatant and 51% of 624.28 cells were killed by CTL in the presence of DV supernatant. See FIG. 6 and FIG. 7.

### Example 14. Analysis of Dengue Virus Activation of Natural Killer Cell Targeting of Cancer Cells

The benchmark for NK-cell killing in the industry is on K562 tumors because they are non-antigen matched. Dengue virus treatment was shown to stimulate NK cells to kill about 100% of the K562s (data not shown).

FEMX and 624.28 tumors are usually much harder for NK cells to kill. 624.28 cells are representative of melanoma cells in advanced cancer, with high HLA and are killed by CTL attack. FEMX cells are melanoma cells with normal expression of HLA A2, which is an inhibitor to lysis by NK-92 cells. Thus, FEMX cells are expected to be resistant to NK attack.

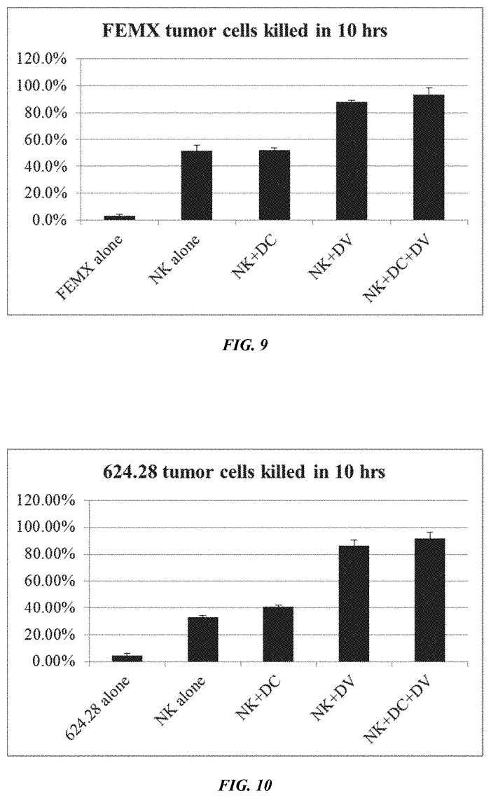

FEMX and 624.28 cancer cell lines were separately co-cultured with NK cells, either in the presence or absence of DV supernatant, and cell death was quantified under each condition. Dengue virus doubled the NK cells' ability to deplete cancer cells, leading to >85% destroyed within 10 hours. In addition, combination of DV and dendritic cells provided for more than 90% killing rates within 10 hours. See FIG. 8 and FIG. 9.

High lysis of DV-activated NK against 624.28 cells and FEMX cells was observed. NK cells killed 33% of 624.28 cells in the presence of mock supernatant and 86% of 624.28 cells in the presence of DV supernatant. NK cells killed 48% of FEMX cells in the presence of mock supernatant and 88% of FEMX cells in the presence of DV supernatant.

### Example 15. Dengue Virus Induced Supernatants from WBCs

DV supernatant was obtained after infecting WBC with Dengue Virus as described in Example 7. The melanoma 624.28 cell line was exposed to the DV supernatant alone (MOI 2) for six hours and cytotoxicity was measured. As controls, 624.28 cells were exposed to cytotoxic T lymphocytes (CTL) alone or mock virus supernatant. FIG. 10 shows the results of this experiment. Treatment of 624.28 cells with DV supematants resulted in about 66% cell death with DV supernatant alone.

### Example 16. Dengue Virus and Dendritic Cells for the Treatment of Melanoma

A Dengue virus (DV) strain (DENV-2 #1710 or DENV-1 #45AZ5) and tumor antigen primed dendritic cells (DCs) a murine model is performed. C57BL/6 mice are inoculated with 0.05 ml of DV at 1×10⁶ or 1×10⁷ pfu/ml by tail vein injection. Recombinant murine IL-2 (Genzyme) and IFN-gamma (Sigma Pharmaceuticals) is administered by intravenous infusion at 2,000 (rIL-2) and 500 1U (rIFN-gamma) on days 5, 10, 15, and 20 following administration of DV. Seven days after the DV administration, C57BL/6 mice are immunized with mouse DCs incubated with the 2 peptides separately and injected intravenously. Peptides were synthesized. The H-2b-restricted peptide from Ovalbumin (OVA-8), SIINFEKL (SEQ ID NO: 7), are used as a control. B16 melanoma-associated H-2b-restricted peptides derived from the antigens gp100/pme117 (EGSRNQDWL (SEQ ID NO: 1)) and from TRP-1/75 (TAYRYHLL (SEQ ID NO: 2)) are used to pulse murine DCs (see Example 1 for details). Two additional immunizations with DCs are given at 14-day intervals. Three days after the last DC infusion, mice were challenged with 5×10⁴ viable B16 melanoma cells intravenously in the lateral tail vein and then followed for survival, which is recorded as the percentage of surviving animals over time (in days) after tumor injection.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treatment or reduction of a melanoma, comprising:
a) administering Dengue virus to a subject in need thereof, wherein the subject has melanoma; and
b) administering primed dendritic cells to the subject, wherein the primed dendritic cells are produced by contacting dendritic cells with a tumor antigen.

2. The method of claim 1, wherein the melanoma is advanced melanoma.

3. The method of claim 1, wherein the melanoma is advanced and is Stage III or Stage IV melanoma.

4. The method of claim 1, comprising obtaining the dendritic cells from the subject at least a week prior to administering the dose of Dengue virus.

5. The method of claim 1, wherein the Dengue virus is administered in an amount between 10⁴ pfu and 10⁸ pfu.

6. The method of claim 1, wherein the Dengue virus is administered in an amount between 10⁵ pfu and 10⁷ pfu.

7. The method of claim 1, wherein the Dengue virus is administered in a concentration of 10,000 PFU/mL to 90,000 PFU/mL.

8. The method of claim 1, wherein the Dengue virus is administered in a concentration of about 30,000 PFU/mL.

9. The method of claim 1, comprising administering primed dendritic cells 4 days to 10 days after administering the dose of Dengue virus.

10. The method of claim 1, wherein the Dengue virus is administered subcutaneously.

11. The method of claim 1, wherein the Dengue virus is administered via intratumoral injection.

12. The method of claim 1, comprising administering primed dendritic cells when the subject presents a febrile symptom.

13. The method of claim 1, comprising administering primed dendritic cells when the subject has reached a temperature of 101° F.

14. The method of claim 1, comprising administering a first aliquot of primed dendritic cells to the subject at a first time and a second aliquot of primed dendritic cells at a second time.

15. The method of claim 12, wherein the first time and the second time are separated by up to 30 days.

16. The method of claim 12, wherein the first time and the second time are separated by about 3 days.

17. The method of claim 12, wherein the number of primed dendritic cells in the first aliquot of primed dendritic cells is 10⁴ cells to 10⁸ cells.

18. The method of claim 12, wherein the total number of primed dendritic cells in each of the first aliquot of primed dendritic cells and second aliquot of primed dendritic cells is 10⁶ cells to 10⁹ cells.

19. The method of claim 1, wherein the dendritic cells are allogeneic to the subject.

20. The method of claim 1, wherein the dendritic cells are autologous to the subject.

21. The method of claim 1, comprising obtaining the dendritic cells from the subject.

22. The method of claim 1, comprising contacting the dendritic cells with tumor lysate from the subject.

23. The method of claim 1, wherein the primed dendritic cells produce at least about 16 ng/mL IL-12p70.

24. The method of claim 1, wherein the primed dendritic cells produce at least about 29 ng/mL IL-12p70.

25. The method of claim 1, wherein the Dengue virus is a serotype 1, 2, 3, 4 or 5.

26. The method of claim 1, wherein the Dengue virus a DENV2 #1710.

27. The method of claim 1, wherein the Dengue virus a DENV1 #45AZ5.

28. The method of claim 1, wherein the Dengue virus is S16803, HON 1991 C, HON 1991 D, HON 1991 B, HON 1991 A, SAL 1987, TRI 1981, PR 1969, IND 1957, TRI 1953, TSV01, DS09-280106, DS31-291005, 1349, GD01/03, 44, 43, China 04, FJ11/99, FJ-10, QHD13CAIQ, CO/BID-V3358, FJ/UH21/1971, GU/BID-V2950, American Asian, GWL18, IN/BID-V2961, Od2112, RR44, 1392, 1016DN, 1017DN, 1070DN, 98900663DHF, BA05i, 1022DN, NGC, Pak-L-2011, Pak-K-2009, Pak-M-2011, PakL-2013, Pak-L-2011, Pak-L-2010, Pak-L-2008, PE/NFI1159, PE/IQA 2080, SG/D2Y98P-PP1, SG/05K3295DK1, LK/BID/V2421, LK/BID-V2422, LK/BID-V2416, 1222-DF-06, TW/BID-V5056, TH/BID-V3357, US/BID-V5412, US/BID-V5055, IQT1797, VN/BID-V735, US/Hawaii/1944, CH53489, or 341750.

29. A method for treatment or reduction of a melanoma, comprising:
a) administering DENV1 #45AZ5 to a subject in need thereof, wherein the subject has melanoma;
b) obtaining dendritic cells from the subject;
c) contacting the dendritic cells with a tumor antigen from the subject to generate primed dendritic cells; and
d) administering the primed dendritic cells to the subject.

30. The method of claim 29, wherein the melanoma is advanced melanoma.

31. The method of claim 29, wherein the melanoma is advanced and is Stage III or Stage IV melanoma.

32. The method of claim 29, wherein the DENV1 #45AZ5 is administered in an amount between 10⁴ pfu and 10⁸ pfu.

33. The method of claim 29, wherein the DENV1 #45AZ5 is administered in an amount between 10⁵ pfu and 10⁷ pfu.

34. The method of claim 29, wherein the DENV1 #45AZ5 is administered in a concentration of 10,000 PFU/mL to 90,000 PFU/mL.

35. The method of claim 29, wherein the DENV1 #45AZ5 is administered in a concentration of about 30,000 PFU/mL.

36. A method for treatment or reduction of a melanoma, comprising:
a) administering DENV2 #1710 to a subject in need thereof, wherein the subject has melanoma;
b) obtaining dendritic cells from the subject;
c) contacting the dendritic cells with a tumor antigen from the subject to generate primed dendritic cells; and
d) administering the primed dendritic cells to the subject.

37. The method of claim 36, wherein the melanoma is advanced melanoma.

38. The method of claim 36, wherein the melanoma is advanced and is Stage III or Stage IV melanoma.

39. The method of claim 36, wherein the DENV2 #1710 is administered in an amount between 10⁴ pfu and 10⁸ pfu.

40. The method of claim 36, wherein the DENV2 #1710 is administered in an amount between 10⁵ pfu and 10⁷ pfu.

41. The method of claim 36, wherein the DENV2 #1710 is administered in a concentration of 10,000 PFU/mL to 90,000 PFU/mL.

42. The method of claim 36, wherein the DENV2 #1710 is administered in a concentration of about 30,000 PFU/mL.


SEQUENCE LISTING (9 sequences)

SEQ ID NO: 1 - protein, 9 aa, Artificial Sequence
  Description of Artificial Sequence Synthetic peptide
EGSRNQDWL

SEQ ID NO: 2 - protein, 8 aa, Artificial Sequence
  Description of Artificial Sequence Synthetic peptide
TAYRYHLL

SEQ ID NO: 3 - DNA, 20 bp, Artificial Sequence
  Description of Artificial Sequence Synthetic primer
ggatcccaagaaggggccat

SEQ ID NO: 4 - DNA, 19 bp, Artificial Sequence
  Description of Artificial Sequence Synthetic primer
ggcagctccatagattgct

SEQ ID NO: 5 - DNA, 19 bp, Artificial Sequence
  Description of Artificial Sequence Synthetic primer
ggtgttgctgcagatggaa

SEQ ID NO: 6 - DNA, 19 bp, Artificial Sequence
  Description of Artificial Sequence Synthetic primer
gtgtcacagacagtgaggt

SEQ ID NO: 7 - protein, 8 aa, Gallus gallus
SIINFEKL

SEQ ID NO: 8 - DNA, 10735 bp, Dengue virus
agttgttagtctacgtggaccgacaagaacagtttcgaatcggaagcttgcttaacgtagttctaacagttttttattagagagcagatctctgatgaacaaccaacggaaaaagacgggtcgaccgtctttcaatatgctgaaacgcgcgagaaaccgcgtgtcaactgtttcacagttggcgaagagattctcaaaaggattgctttcaggccaaggacccatgaaattggtgatggcttttatagcattcctaagatttctagccatacctccaacagcaggaattttggctagatggggctcattcaagaagaatggagcgatcaaagtgttacggggtttcaagaaagaaatctcaaacatgttgaacataatgaacaggaggaaaagatctgtgaccatgctcctcatgctgctgcccacagccctggcgttccatctgaccacccgagggggagagccgcacatgatagttagcaagcaggaaagaggaaaatcacttttgtttaagacctctgcaggtgtcaacatgtgcacccttattgcaatggatttgggagagttatgtgaggacacaatgacctacaaatgcccccggatcactgagacggaaccagatgacgttgactgttggtgcaatgccacggagacatgggtgacctatggaacatgttctcaaactggtgaacaccgacgagacaaacgttccgtcgcactggcaccacacgtagggcttggtctagaaacaagaaccgaaacgtggatgtcctctgaaggcgcttggaaacaaatacaaaaagtggagacctgggctctgagacacccaggattcacggtgatagccctttttctagcacatgccataggaacatccatcacccagaaagggatcatttttattttgctgatgctggtaactccatccatggccatgcggtgcgtgggaataggcaacagagacttcgtggaaggactgtcaggagctacgtgggtggatgtggtactggagcatggaagttgcgtcactaccatggcaaaagacaaaccaacactggacattgaactcttgaagacggaggtcacaaaccctgccgtcctgcgcaaactgtgcattgaagctaaaatatcaaacaccaccaccgattcgagatgtccaacacaaggagaagccacgctggtggaagaacaggacacgaactttgtgtgtcgacgaacgttcgtggacagaggctggggcaatggttgtgggctattcggaaaaggtagcttaataacgtgtgctaagtttaagtgtgtgacaaaactggaaggaaagatagtccaatatgaaaacttaaaatattcagtgatagtcaccgtacacactggagaccagcaccaagttggaaatgagaccacagaacatggaacaactgcaaccataacacctcaagctcccacgtcggaaatacagctgacagactacggagctctaacattggattgttcacctagaacagggctagactttaatgagatggtgttgttgacaatgaaaaaaaaatcatggctcgtccacaaacaatggtttctagacttaccactgccttggacctcgggggcttcaacatcccaagagacttggaatagacaagacttgctggtcacatttaagacagctcatgcaaaaaagcaggaagtagtcgtactaggatcacaagaaggagcaatgcacactgcgttgactggagcgacagaaatccaaacgtctggaacgacaacaatttttgcaggacacctgaaatgcagattaaaaatggataaactgattttaaaagggatgtcatatgtaatgtgcacagggtcattcaagttagagaaggaagtggctgagacccagcatggaactgttctagtgcaggttaaatacgaaggaacagatgcaccatgcaagatccccttctcgtcccaagatgagaagggagtaacccagaatgggagattgataacagccaaccccatagtcactgacaaagaaaaaccagtcaacattgaagcggagccaccttttggtgagagctacattgtggtaggagcaggtgaaaaagctttgaaactaagctggttcaagaagggaagcagtatagggaaaatgtttgaagcaactgcccgtggagcacgaaggatggccatcctgggagacactgcatgggacttcggttctataggaggggtgttcacgtctgtgggaaaactgatacaccagatttttgggactgcgtatggagttttgttcagcggtgtttcttggaccatgaagataggaatagggattctgctgacatggctaggattaaactcaaggagcacgtccctttcaatgacgtgtatcgcagttggcatggtcacactgtacctaggagtcatggttcaggcggactcgggatgtgtaatcaactggaaaggcagagaactcaaatgtggaagcggcatttttgtcaccaatgaagtccacacctggacagagcaatataaattccaggccgactcccctaagagactatcagcggccattgggaaggcatgggaggagggtgtgtgtggaattcgatcagccactcgtctcgagaacatcatgtggaagcaaatatcaaatgaattaaaccacatcttacttgaaaatgacatgaaatttacagtggtcgtaggagacgttagtggaatcttggcccaaggaaagaaaatgattaggccacaacccatggaacacaaatactcgtggaaaagctggggaaaagccaaaatcataggagcagatgtacagaataccaccttcatcatcgacggcccaaacaccccagaatgccctgataaccaaagagcatggaacatttgggaagttgaagactatggatttggaattttcacgacaaacatatggttgaaattgcgtgactcctacactcaagtgtgtgaccaccggctaatgtcagctgccatcaaggatagcaaagcagtccatgctgacatggggtactggatagaaagtgaaaagaacgagacttggaagttggcaagagcctccttcatagaagttaagacatgcatctggccaaaatcccacactctatggagcaatggagtcctggaaagtgagatgataatcccaaagatatatggaggaccaatatctcagcacaactacagaccaggatatttcacacaaacagcagggccgtggcacttgggcaagttagaactagattttgatttatgtgaaggtaccactgttgttgtggatgaacattgtggaaatcgaggaccatctcttagaaccacaacagtcacaggaaagacaatccatgaatggtgctgtagatcttgcacgttaccccccctacgtttcaaaggagaagacgggtgctggtacggcatggaaatcagaccagtcaaggagaaggaagagaacctagttaagtcaatggtctctgcagggtcaggagaagtggacagtttttcactaggactgctatgcatatcaataatgatcgaagaggtaatgagatccagatggagcagaaaaatgctgatgactggaacattggctgtgttcctccttctcacaatgggacaattgacatggaatgatctgatcaggctatgtatcatggttggagccaacgcttcagacaagatggggatgggaacaacgtacctagctttgatggccactttcagaatgagaccaatgttcgcagtcgggctactgtttcgcagattaacatctagagaagttcttcttcttacagttggattgagtctggtggcatctgtagaactaccaaattccttagaggagctaggggatggacttgcaatgggcatcatgatgttgaaattactgactgattttcagtcacatcagctatgggctaccttgctgtctttaacatttgtcaaaacaactttttcattgcactatgcatggaagacaatggctatgatactgtcaattgtatctctcttccctttatgcctgtccacgacttctcaaaaaacaacatggcttccggtgttgctgggatctcttggatgcaaaccactaaccatgtttcttataacagaaaacaaaatctggggaaggaaaagctggcctctcaatgaaggaattatggctgttggaatagttagcattcttctaagttcacttctcaagaatgatgtgccactagctggcccactaatagctggaggcatgctaatagcatgttatgtcatatctggaagctcggccgatttatcactggagaaagcggctgaggtctcctgggaagaagaagcagaacactctggtgcctcacacaacatactagtggaggtccaagatgatggaaccatgaagataaaggatgaagagagagatgacacactcaccattctcctcaaagcaactctgctagcaatctcaggggtatacccaatgtcaataccggcgaccctctttgtgtggtatttttggcagaaaaagaaacagagatcaggagtgctatgggacacacccagccctccagaagtggaaagagcagtccttgatgatggcatttatagaattctccaaagaggattgttgggcaggtctcaagtaggagtaggagtttttcaagaaggcgtgttccacacaatgtggcacgtcaccaggggagctgtcctcatgtaccaagggaagagactggaaccaagttgggccagtgtcaaaaaagacttgatctcatatggaggaggttggaggtttcaaggatcctggaacgcgggagaagaagtgcaggtgattgctgttgaaccggggaagaaccccaaaaatgtacagacagcgccgggtaccttcaagacccctgaaggcgaagttggagccatagctctagactttaaacccggcacatctggatctcctatcgtgaacagagagggaaaaatagtaggtctttatggaaatggagtggtgacaacaagtggtacctacgtcagtgccatagctcaagctaaagcatcacaagaagggcctctaccagagattgaggacgaggtgtttaggaaaagaaacttaacaataatggacctacatccaggatcgggaaaaacaagaagataccttccagccatagtccgtgaggccataaaaagaaagctgcgcacgctagtcttagctcccacaagagttgtcgcttctgaaatggcagaggcgctcaagggaatgccaataaggtatcagacaacagcagtgaagagtgaacacacgggaaaggagatagttgaccttatgtgtcacgccactttcactatgcgtctcctgtctcctgtgagagttcccaattataatatgattatcatggatgaagcacattttaccgatccagccagcatagcagccagagggtatatctcaacccgagtgggtatgggtgaagcagctgcgattttcatgacagccactccccccggatcggtggaggcctttccacagagcaatgcagttatccaagatgaggaaagagacattcctgaaagatcatggaactcaggctatgactggatcactgatttcccaggtaaaacagtctggtttgttccaagcatcaaatcaggaaatgacattgccaactgtttaagaaagaatgggaaacgggtggtccaattgagcagaaaaacttttgacactgagtaccagaaaacaaaaaataacgactgggactatgttgtcacaacagacatatccgaaatgggagcaaacttccgagccgacagggtaatagacccgaggcggtgcctgaaaccggtaatactaaaagatggcccagagcgtgtcattctagccggaccgatgccagtgactgtggctagcgccgcccagaggagaggaagaattggaaggaaccaaaataaggaaggcgatcagtatatttacatgggacagcctctaaacaatgatgaggaccacgcccattggacagaagcaaaaatgctccttgacaacataaacacaccagaagggattatcccagccctctttgagccggagagagaaaagagtgcagcaatagacggggaatacagactacggggtgaagcgaggaaaacgttcgtggagctcatgagaagaggagatctacctgtctggctatcctacaaagttgcctcagaaggcttccagtactccgacagaaggtggtgctttgatggggaaaggaacaaccaggtgttggaggagaacatggacgtggagatctggacaaaagaaggagaaagaaagaaactacgaccccgctggctggatgccagaacatactctgacccactggctctgcgcgaattcaaagagttcgcagcaggaagaagaagcgtctcaggtgacctaatattagaaatagggaaacttccacaacatttaacgcaaagggcccagaacgccttggacaatctggttatgttgcacaactctgaacaaggaggaaaagcctatagacacgccatggaagaactaccagacaccatagaaacgttaatgctcctagctttgatagctgtgctgactggtggagtgacgttgttcttcctatcaggaaggggtctaggaaaaacatccattggcctactctgcgtgattgcctcaagtgcactgttatggatggccagtgtggaaccccattggatagcggcctctatcatactggagttctttctgatggtgttgcttattccagagccggacagacagcgcactccacaagacaaccagctagcatacgtggtgataggtctgttattcatgatattgacagtggcagccaatgagatgggattactggaaaccacaaagaaggacctggggattggtcatgcagctgctgaaaaccaccatcatgctgcaatgctggacgtagacctacatccagcttcagcctggactctctatgcagtggccacaacaattatcactcccatgatgagacacacaattgaaaacacaacggcaaatatttccctgacagctattgcaaaccaggcagctatattgatgggacttgacaagggatggccaatatcaaagatggacataggagttccacttctcgccttggggtgctattctcaggtgaacccgctgacgctgacagcggcggtattgatgctagtggctcattatgccataattggacccggactgcaagcaaaagctactagagaagctcaaaaaaggacagcagccggaataatgaaaaacccaactgtcgacgggatcgttgcaatagatttggaccctgtggtttacgatgcaaaatttgaaaaacagctaggccaaataatgttgttgatactttgcacatcacagatcctcctgatgcggaccacatgggccttgtgtgaatccatcacactagccactggacctctgactacgctttgggagggatctccaggaaaattctggaacaccacgatagcggtgtccatggcaaacatttttaggggaagttatctagcaggagcaggtctggccttttcattaatgaaatctctaggaggaggtaggagaggcacgggagcccaaggggaaacactgggagaaaaatggaaaagacagctaaaccaattgagcaagtcagaattcaacacttacaaaaggagtgggattatagaggtggatagatctgaagccaaagaggggttaaaaagaggagaaacgactaaacacgcagtgtcgagaggaacggccaaactgaggtggtttgtggagaggaaccttgtgaaaccagaagggaaagtcatagacctcggttgtggaagaggtggctggtcatattattgcgctgggctgaagaaagtcacagaagtgaaaggatacacgaaaggaggacctggacatgaggaaccaatcccaatggcaacctatggatggaacctagtaaagctatactccgggaaagatgtattctttacaccacctgagaaatgtgacaccctcttgtgtgatattggtgagtcctctccgaacccaactatagaagaaggaagaacgttacgtgttctaaagatggtggaaccatggctcagaggaaaccaattttgcataaaaattctaaatccctatatgccgagtgtggtagaaactttggagcaaatgcaaagaaaacatggaggaatgctagtgcgaaatccactctcaagaaactccactcatgaaatgtactgggtttcatgtggaacaggaaacattgtgtcagcagtaaacatgacatctagaatgctgctaaatcgattcacaatggctcacaggaagccaacatatgaaagagacgtggacttaggcgctggaacaagacatgtggcagtagaaccagaggtggccaacctagatatcattggccagaggatagagaatataaaaaatgaacacaaatcaacatggcattatgatgaggacaatccatacaaaacatgggcctatcatggatcatatgaggtcaagccatcaggatcagcctcatccatggtcaatggtgtggtgagactgctaaccaaaccatgggatgtcattcccatggtcacacaaatagccatgactgacaccacaccctttggacaacagagggtgtttaaagagaaagttgacacgcgtacaccaaaagcgaaacgaggcacagcacaaattatggaggtgacagccaggtggttatggggttttctctctagaaacaaaaaacccagaatctgcacaagagaggagttcacaagaaaagtcaggtcaaacgcagctattggagcagtgttcgttgatgaaaatcaatggaactcagcaaaagaggcagtggaagatgaacggttctgggaccttgtgcacagagagagggagcttcataaacaaggaaaatgtgccacgtgtgtctacaacatgatgggaaagagagagaaaaaattaggagagttcggaaaggcaaaaggaagtcgcgcaatatggtacatgtggttgggagcgcgctttttagagtttgaagcccttggtttcatgaatgaagatcactggttcagcagagagaattcactcagtggagtggaaggagaaggactccacaaacttggatacatactcagagacatatcaaagattccagggggaaatatgtatgcagatgacacagccggatgggacacaagaataacagaggatgatcttcagaatgaggccaaaatcactgacatcatggaacctgaacatgccctattggccacgtcaatctttaagctaacctaccaaaacaaggtagtaagggtgcagagaccagcgaaaaatggaaccgtgatggatgtcatatccagacgtgaccagagaggaagtggacaggttggaacctatggcttaaacaccttcaccaacatggaggcccaactaataagacaaatggagtctgagggaatcttttcacccagcgaattggaaaccccaaatctagccgaaagagtcctcgactggttgaaaaaacatggcaccgagaggctgaaaagaatggcaatcagtggagatgactgtgtggtgaaaccaatcgatgacagatttgcaacagccttaacagctttgaatgacatgggaaaggtaagaaaagacataccgcaatgggaaccttcaaaaggatggaatgattggcaacaagtgcctttctgttcacaccatttccaccagctgattatgaaggatgggagggagatagtggtgccatgccgcaaccaagatgaacttgtaggtagggccagagtatcacaaggcgccggatggagcttgagagaaactgcatgcctaggcaagtcatatgcacaaatgtggcagctgatgtacttccacaggagagacttgagattagcggctaatgctatctgttcagccgttccagttgattgggtcccaaccagccgcaccacctggtcgatccatgcccaccatcaatggatgacaacagaagacatgttgtcagtgtggaatagggtttggatagaggaaaacccatggatggaggacaagactcatgtgtccagttgggaagacgttccatacctaggaaaaagggaagatcaatggtgtggttccctaataggcttaacagcacgagccacctgggccaccaacatacaagtggccataaaccaagtgagaaggctcattgggaatgagaattatctagacttcatgacatcaatgaagagattcaaaaacgagagtgatcccgaaggggcactctggtaagccaactcattcacaaaataaaggaaaataaaaaatcaaacaaggcaagaagtcaggccggattaagccatagcacggtaagagctatgctgcctgtgagccccgtccaaggacgtaaaatgaagtcaggccgaaagccacggttcgagcaagccgtgctgcctgtagctccatcgtggggatgtaaaaacccgggaggctgcaaaccatggaagctgtacgcatggggtagcagactagtggttagaggagacccctcccaagacacaacgcagcagcggggcccaacaccaggggaagctgtaccctggtggtaaggactagaggttagaggagaccccccgcacaacaacaaacagcatattgacgctgggagagaccagagatcctgctgtctctacagcatcattccaggcacagaacgccaaaaaatggaatggtgctgttgaatcaacaggttct

SEQ ID NO: 9 - protein, 3392 aa, Dengue virus
MNNQRKKTGRPSFNMLKRARNRVSTVSQLAKRFSKGLLSGQGPMKLVMAFIAFLRFLAIPPTAGILARWGSFKKNGAIKVLRGFKKEISNMLNIMNRRKRSVTMLLMLLPTALAFHLTTRGGEPHMIVSKQERGKSLLFKTSAGVNMCTLIAMDLGELCEDTMTYKCPRITETEPDDVDCWCNATETWVTYGTCSQTGEHRRDKRSVALAPHVGLGLETRTETWMSSEGAWKQIQKVETWALRHPGFTVIALFLAHAIGTSITQKGIIFILLMLVTPSMAMRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMKKKSWLVHKQWFLDLPLPWTSGASTSQETWNRQDLLVTFKTAHAKKQEVVVLGSQEGAMHTALTGATEIQTSGTTTIFAGHLKCRLKMDKLILKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQADSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIRPQPMEHKYSWKSWGKAKIIGADVQNTTFIIDGPNTPECPDNQRAWNIWEVEDYGFGIFTTNIWLKLRDSYTQVCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDLCEGTTVVVDEHCGNRGPSLRTTTVTGKTIHEWCCRSCTLPPLRFKGEDGCWYGMEIRPVKEKEENLVKSMVSAGSGEVDSFSLGLLCISIMIEEVMRSRWSRKMLMTGTLAVFLLLTMGQLTWNDLIRLCIMVGANASDKMGMGTTYLALMATFRMRPMFAVGLLFRRLTSREVLLLTVGLSLVASVELPNSLEELGDGLAMGIMMLKLLTDFQSHQLWATLLSLTFVKTTFSLHYAWKTMAMILSIVSLFPLCLSTTSQKTTWLPVLLGSLGCKPLTMFLITENKIWGRKSWPLNEGIMAVGIVSILLSSLLKNDVPLAGPLIAGGMLIACYVISGSSADLSLEKAAEVSWEEEAEHSGASHNILVEVQDDGTMKIKDEERDDTLTILLKATLLAISGVYPMSIPATLFVWYFWQKKKQRSGVLWDTPSPPEVERAVLDDGIYRILQRGLLGRSQVGVGVFQEGVFHTMWHVTRGAVLMYQGKRLEPSWASVKKDLISYGGGWRFQGSWNAGEEVQVIAVEPGKNPKNVQTAPGTFKTPEGEVGAIALDFKPGTSGSPIVNREGKIVGLYGNGVVTTSGTYVSAIAQAKASQEGPLPEIEDEVFRKRNLTIMDLHPGSGKTRRYLPAIVREAIKRKLRTLVLAPTRVVASEMAEALKGMPIRYQTTAVKSEHTGKEIVDLMCHATFTMRLLSPVRVPNYNMIIMDEAHFTDPASIAARGYISTRVGMGEAAAIFMTATPPGSVEAFPQSNAVIQDEERDIPERSWNSGYDWITDFPGKTVWFVPSIKSGNDIANCLRKNGKRVVQLSRKTFDTEYQKTKNNDWDYVVTTDISEMGANFRADRVIDPRRCLKPVILKDGPERVILAGPMPVTVASAAQRRGRIGRNQNKEGDQYIYMGQPLNNDEDHAHWTEAKMLLDNINTPEGIIPALFEPEREKSAAIDGEYRLRGEARKTFVELMRRGDLPVWLSYKVASEGFQYSDRRWCFDGERNNQVLEENMDVEIWTKEGERKKLRPRWLDARTYSDPLALREFKEFAAGRRSVSGDLILEIGKLPQHLTQRAQNALDNLVMLHNSEQGGKAYRHAMEELPDTIETLMLLALIAVLTGGVTLFFLSGRGLGKTSIGLLCVIASSALLWMASVEPHWIAASIILEFFLMVLLIPEPDRQRTPQDNQLAYVVIGLLFMILTVAANEMGLLETTKKDLGIGHAAAENHHHAAMLDVDLHPASAWTLYAVATTIITPMMRHTIENTTANISLTAIANQAAILMGLDKGWPISKMDIGVPLLALGCYSQVNPLTLTAAVLMLVAHYAIIGPGLQAKATREAQKRTAAGIMKNPTVDGIVAIDLDPVVYDAKFEKQLGQIMLLILCTSQILLMRTTWALCESITLATGPLTTLWEGSPGKFWNTTIAVSMANIFRGSYLAGAGLAFSLMKSLGGGRRGTGAQGETLGEKWKRQLNQLSKSEFNTYKRSGIIEVDRSEAKEGLKRGETTKHAVSRGTAKLRWFVERNLVKPEGKVIDLGCGRGGWSYYCAGLKKVTEVKGYTKGGPGHEEPIPMATYGWNLVKLYSGKDVFFTPPEKCDTLLCDIGESSPNPTIEEGRTLRVLKMVEPWLRGNQFCIKILNPYMPSVVETLEQMQRKHGGMLVRNPLSRNSTHEMYWVSCGTGNIVSAVNMTSRMLLNRFTMAHRKPTYERDVDLGAGTRHVAVEPEVANLDIIGQRIENIKNEHKSTWHYDEDNPYKTWAYHGSYEVKPSGSASSMVNGVVRLLTKPWDVIPMVTQIAMTDTTPFGQQRVFKEKVDTRTPKAKRGTAQIMEVTARWLWGFLSRNKKPRICTREEFTRKVRSNAAIGAVFVDENQWNSAKEAVEDERFWDLVHRERELHKQGKCATCVYNMMGKREKKLGEFGKAKGSRAIWYMWLGARFLEFEALGFMNEDHWFSRENSLSGVEGEGLHKLGYILRDISKIPGGNMYADDTAGWDTRITEDDLQNEAKITDIMEPEHALLATSIFKLTYQNKVVRVQRPAKNGTVMDVISRRDQRGSGQVGTYGLNTFTNMEAQLIRQMESEGIFSPSELETPNLAERVLDWLKKHGTERLKRMAISGDDCVVKPIDDRFATALTALNDMGKVRKDIPQWEPSKGWNDWQQVPFCSHHFHQLIMKDGREIVVPCRNQDELVGRARVSQGAGWSLRETACLGKSYAQMWQLMYFHRRDLRLAANAICSAVPVDWVPTSRTTWSIHAHHQWMTTEDMLSVWNRVWIEENPWMEDKTHVSSWEDVPYLGKREDQWCGSLIGLTARATWATNIQVAINQVRRLIGNENYLDFMTSMKRFKNESDPEGALW